(19) United States
(12) Patent Application Publication
(10) Pub. No.: US 2011/0021442 A1
(43) Pub. Date: Jan. 27, 2011

(54) CELL PREAMBLE RUNX3 RECOMBINANT PROTEINS, POLYNUCLEOTIDES ENCODING THE SAME, AND ANTICANCER COMPOSITIONS INCLUDING THE SAME

(76) Inventors: Daewoong Jo, Seoul (KR); Eun Kyung Hong, Gwangju (KR); Jung-Hee Lim, Seoul (KR); Se-Eun Kang, Busan (KR); Lihua Che, Incheon (KR)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/741,138

(22) PCT Filed: Nov. 6, 2008
(86) PCT No.: PCT/KR2008/006526

§ 371 Date: May 3, 2010

Related U.S. Application Data
(60) Provisional application No. 60/985,765, filed on Nov. 6, 2007.

(51) Int. Cl. A61K 38/00; C07K 14/00; C07H 21/04; C12N 15/63; C12N 1/21; C12P 21/06; A61P 35/00; A61P 35/04

(52) U.S. Cl. 514/19.8; 530/350; 536/23.1; 435/320.1; 435/252.33; 435/69.1; 514/19.3

(57) ABSTRACT

The present invention discloses cell permeable RUNX3 recombinant proteins where a Macromolecule Transduction Domain (MTD) is fused to a tumor and metastasis suppressor RUNX3. Also disclosed are polynucleotides encoding the cell permeable RUNX3 recombinant proteins, an expression vector containing the cell permeable RUNX3 recombinant protein, and a pharmaceutical composition for preventing metastasis which contains the cell permeable RUNX3 recombinant protein as an effective ingredient. The cell permeable RUNX3 recombinant proteins of the present invention can induce the reactivation of TGF-β signal transduction pathway which causes cell cycle arrest by efficiently introducing a tumor and metastasis suppressor RUNX3 into a cell. Therefore, the cell permeable RUNX3 recombinant proteins of the present invention can be effectively used as an anticancer agent capable of preventing cancer growth and metastasis by suppressing the proliferation, differentiation, and migration of cancer cells.

### TECHNICAL FIELD

The present invention relates to cell permeable RUNX3 recombinant proteins in which a tumor and metastasis suppressor RUNX3 is fused to a macromolecule transduction domain (MTD), polynucleotides encoding the same, expression vectors for producing the same, and anticancer pharmaceutical compositions including the same as effective ingredients for treating RUNX3 deficiency or failure.

### BACKGROUND ART

Gastric cancer is the most common cancer in Asian countries (e.g., Korea, Japan) and is the second most fatal disease worldwide. Therefore, it is very important to diagnose gastric cancer in its early stage. However, in the early stages of stomach cancer, the symptoms are vague and there is no characteristic symptom, making gastric cancer tricky to diagnose. Thus, a great deal of research on developing a fundamental treatment for gastric cancer through a comprehensive understanding of its pathogenesis has been actively carried out.

Recently, it has been reported that the reduction in expression of Runt-related transcription factor, RUNX3 relates to cancer development in the stomach (Li et al., Cell 109: 113-124, 2002). According to that report, a mouse model designed to have RUNX3 deficiency or failure in order to identify the function of RUNX3 developed gastric cancer due to the RUNX3 mutation.

Generally, if normal cells become old and diseased, they will die and new cells are generated on the spot. In RUNX3-deficient mice, these abnormal cells proliferate permanently, resulting in hyperplasia due to an increase in cell proliferation and a decrease in cell death. However, p53-deficient mice with normal RUNX3 did not develop gastric cancer (Li et al., Cell 109: 113-124, 2002). These results suggest that RUNX3 plays an important role in regulating cell proliferation and that the inactivation of RUNX3 may be a potential cause of gastric cancer. In fact, based on an analysis of the examination of gastric cancer cells and tissues to confirm the inhibitory effect of RUNX3 on gastric cancer, a close relationship has been found to exist between RUNX3 and gastric cancer. In particular, in analyzing tissues of 46 gastric cancer patients, hemizygous deletion of RUNX3 was detected in 30% of the patients, where RUNX3 was inactivated in about 45˜60% of those patients due to hypermethylation of CpG islands located at the RUNX3 promoter (Li et al., Cell 109: 113-124, 2002; Waki et al., Cancer Sci. 94: 360-364, 2003).

The RUNT-domain family of transcription factors known as polyomavirus enhancer binding protein 1/core binding factors (PEBP2/CBF) is composed of RUNX1 (PEBP2αB/CBFA2/AML1), RUNX2 (PEBP2αA/CBFA1/AML3) and RUNX3 (PEBP2αC/CBFA3/AML2). The RUNT-domain family is a key player in normal development and oncogenesis and, for instance, functions as a transcription factor for the Smad family which is a subunit capable of mediating TGF-β and the signal transduction thereof. RUNX1 is essential for definitive haematopoiesis in mammals, while RUNX2 promotes osteogenesis and cell differentiation and RUNX3 mainly expressed in granular gastric mucous cells functions to inhibit epithelial cell differentiation. These three members are located on chromosomes 1p, 6p, and 21 q, respectively, and the chromosomal locus of RUNX3 is 1p36.11-1p36.13. The RUNX3 locus is commonly deleted in a variety of human cancers, including gastric cancer, pancreatic cancer, lung cancer, colon cancer, liver cancer and the like, and is a site that is easily subject to hemizygous deletion. Further, it has been found that RUNX3 is inactivated in a number of the above listed human cancers, suggesting that RUNX3 is a promising target for the development of a new anticancer drug.

It has been also reported that RUNX3 is capable of not only inhibiting tumor growth as a tumor suppressor but also suppressing metastasis. RUNX3 inhibits the expression of vascular endothelial growth factor (VEGF) which is involved in the formation of blood vessels essential for cancer metastasis (Keping Xie et al., Cancer Res. 65:4809-5816, 2006), while cancer metastasis in a RUNX3-transgenic mouse is further suppressed as compared with a control (Hagiwara et al., Clin Cancer Res. 11(18): 6479-6488, 2005).

When RUNX3 stimulates a signal transduction pathway of TGF-β, the thus stimulated TGF-β induces the activation of Smad2/3. After the TGF-β-induced activation, Smad2/3 interacts with Smad4 and transfers into the nucleus in a complex form, followed by binding to p300 and RUNX3. Consequently, the transcription of a target gene is induced and apoptosis occurrs.

It has been known that TGF-β is involved in many development processes and physiological activities as a cell growth regulator. A TGF-β receptor and its signal transduction protein Smad are usually inactivated in various different cancers (Cohen et al., Am. J. Med. Genet. 116A: 1-10, 2003). It has also been reported that p300 involved in the TGF-β signal transduction pathway, in combination with Smad, is mutated in a variety of cancers (Gayther et al., Nat. Genet. 24: 300-303. 2000). RUNX3 present in the nucleus interacts with both Smad and p300 involved in the TGF-β signal transduction pathway and cooperatively acts as a tumor and metastasis suppressor (Hanai et al., J. Biol. Chem. 274: 31577-1582. 1999; Kitabayashi et al., EMBO J. 17: 2994-3004. 1998; Lee et al., Mol. Cell. Biol. 20: 8783-8792, 2000; Zhang et al., Proc. Natl. Acad. Sci. USA. 97: 10549-10554, 2000).

TGF-β also inhibits cell proliferation by blocking the G1 phase of the cell cycle (Sherr et al., Science 274: 1672-677, 1996; Weinberg et al., Cell 81: 323-30, 1995). When RUNX3 that has gone through the TGF-β signal transduction pathway forms a complex with Smad2, Smad4, p300, and the like in the nucleus while the expression of p21 which inhibits the cell cycle increases, the phosphorylation of Cyclin A, Cyclin E, PCNA, and Rb regulating the cell cycle, as well as the expression of VEGF responsible for metastasis, is suppressed, leading to the inhibition of metastasis.

Thus considering that it would be possible to effectively suppress tumor growth and metastasis if the overexpression of RUNX3 is induced in vivo or RUNX3 is directly delivered into the cells, the present inventors endeavored to develop new anticancer agents by using the RUNX3 protein.

Meanwhile, small molecules derived from synthetic compounds or natural compounds can be transported into the cells, whereas macromolecules, such as proteins, peptides, and nucleic acids, cannot. It is widely understood that macromolecules larger than 500 kDa are incapable of penetrating the plasma membrane, i.e., the lipid bilayer structure, of live cells. To overcome this problem, a “macromolecule intracellular transduction technology (MITT)” was developed (Jo et al., Nat. Biotech. 19: 929-33, 2001), which allows the delivery of therapeutically effective macromolecules into cells, making the development of new drugs using peptides, proteins and genetic materials possible. According to this method, if a target macromolecule is fused to a hydrophobic macromolecule transduction domain (MTD) and other cellular delivery regulators, synthesized, expressed, and purified in the form of a recombinant protein, it can penetrate the plasma membrane lipid bilayer of the cells, be accurately delivered to a target site, and then, effectively exhibit its therapeutic effect. Such MTDs facilitate the transport of many impermeable materials which are fused to peptides, proteins, DNA, RNA, synthetic compounds, and the like into the cells.

Accordingly, the inventors of the present invention have developed a method of mediating the transport of a tumor and metastasis suppressor RUNX3 into the cells, where cell permeable RUNX3 recombinant proteins are engineered by fusing a MTD to the tumor and metastasis suppressor RUNX3. Such cell permeable RUNX3 recombinant proteins have been found to efficiently mediate the transport of the tumor and metastasis suppressor RUNX3 into the cells in vivo as well as in vitro and can be used as anticancer agents for inhibiting metastasis occurring in various human cancers.

### DISCLOSURE

### Technical Problem

Accordingly, the objective of the present invention is to provide cell permeable RUNX3 recombinant proteins effective for the treatment of RUNX3 deficiency or failure occurring in various kinds of human cancers as anticancer agents.

### Technical Solution

One aspect of the present invention relates to cell permeable RUNX3 recombinant proteins capable of mediating the transport of a tumor and metastasis suppressor RUNX3 into a cell by fusing a macromolecule transduction domain (MTD) having cell permeability to the tumor and metastasis suppressor protein.

Another aspect of the present invention relates to polynucleotides encoding the above cell permeable RUNX3 recombinant proteins.

The present invention also relates to expression vectors containing the above polynucleotides, and transformants transformed with the above expression vectors.

Another aspect of the present invention relates to a method of producing cell permeable RUNX3 recombinant proteins involving culturing the above transformants.

Another aspect of the present invention relates to a pharmaceutical composition including the above cell permeable RUNX3 recombinant proteins as an effective ingredient for treating RUNX3 deficiency or failure.

### INDUSTRIAL APPLICABILITY

The cell permeable RUNX3 recombinant proteins of the present invention can induce the reactivation of TGF-β signal transduction pathway which causes cell cycle arrest by efficiently introducing a tumor and metastasis suppressor RUNX3 into a cell. Therefore, the cell permeable RUNX3 recombinant proteins of the present invention can be effectively used as an anticancer agent capable of preventing cancer growth and metastasis by suppressing the proliferation, differentiation, and migration of cancer cells.

### DESCRIPTION OF DRAWINGS

FIG. 1a is a schematic diagram illustrating the structures of cell permeable RUNX3 recombinant proteins being fused to a kFGF4-derived MTD and constructed in the full-length and truncated forms according to the present invention.

FIG. 1b is a schematic diagram illustrating the structures of cell permeable RUNX3 recombinant proteins being fused to one of JO-57, JO-85, JO-13 and JO-108 MTDs, and constructed in the full-length form according to the present invention.

FIG. 2a is a photograph of an agarose gel electrophoresis analysis showing PCR-amplified DNA fragments encoding cell permeable RUNX3 recombinant proteins being fused to a kFGF4-derived MTD and constructed in the full-length and truncated forms according to the present invention.

FIG. 2b is a photograph of an agarose gel electrophoresis analysis showing PCR-amplified DNA fragments encoding cell permeable RUNX3 recombinant proteins being fused to one of JO-57, JO-85, JO-13 and JO-108 MTDs, and constructed in the full-length and truncated forms according to the present invention.

FIG. 3a is a schematic diagram illustrating the subcloning of a PCR product encoding a cell permeable RUNX3 recombinant protein into the pGEM-T Easy vector according to the present invention.

FIGS. 3b and 3c are photographs of an agarose gel electrophoresis analysis showing the PCR products encoding the cell permeable RUNX3 recombinant proteins subcloned in the pGEM-T Easy vector according to the present invention, respectively.

FIG. 4a is a schematic diagram illustrating the cloning of a recombinant DNA fragment encoding a cell permeable RUNX3 recombinant protein into the pET-28(+) vector according to the present invention.

FIGS. 4b and 4c are photographs of an agarose gel electrophoresis analysis showing the recombinant DNA fragments encoding the cell permeable RUNX3 recombinant proteins subcloned in the pET-28(+) vector according to the present invention, respectively.

FIG. 5a is a photograph of a SDS-PAGE analysis showing the inducible expression of cell permeable RUNX3 recombinant proteins according to the present invention in various kinds of host cells.

FIG. 5b is a photograph of a SDS-PAGE analysis showing the inducible expression of cell permeable RUNX3 recombinant proteins according to the present invention in the presence (+) or the absence (−) of IPTG as an inducer.

FIGS. 6a and 6b are photographs of a SDS-PAGE analysis showing the purification of cell permeable RUNX3 recombinant proteins (HM₁R3, HR3M₁, HM₁R3M₁, HM₂R3 and HM₃R3) expressed from the transformants where the expression vector according to the present invention is transformed into.

FIGS. 7a and 7b are graphs illustrating the results of flow cytometry analysis of cell permeabilities of cell permeable RUNX3 recombinant proteins (HM₁R3, HR3M₁, HM₁R3M₁ and HM₃R3) according to the present invention.

FIG. 8 is a confocal laser scanning microscopy photograph visualizing the cell permeabilities of cell permeable RUNX3 recombinant proteins (HM₁R3, HR3M₁, HM₁R3M₁, HM₂R3 and HM₃R3) according to the present invention in mouse fibroblasts.

FIG. 9 is a confocal laser scanning microscopy photograph visualizing the cell permeabilities of cell permeable Nm23 recombinant protein (HM₃R3) according to the present invention in various kinds of mouse tissues.

FIGS. 10a and 10b are photographs of a Western blot analysis showing the in vivo function of cell permeable RUNX3 recombinant proteins (HM₁R3M₁, HM₂R3 and HM₃R3) according to the present invention.

FIG. 11 is a photograph of a cellular DNA content analysis showing the apoptosis-inducing effect of cell permeable RUNX3 recombinant proteins (HM₁R3M₁, HM₂R3 and HM₃R3) according to the present invention.

FIGS. 12a and 12b are photographs of a wound healing assay showing the inhibitory effect of cell permeable RUNX3 recombinant proteins (HM₁R3M₁, HM₂R3 and HM₃R3) according to the present invention on tumor cell migration.

FIGS. 13a and 13b are graphs illustrating the change in tumor size and body weight, respectively, in a tumor-bearing mouse where each of cell permeable RUNX3 recombinant proteins (HM₂R3 and HM₃R3) according to the present invention was administered via subcutaneous injection for 26 days.

FIG. 14 is a photograph illustrating the change in tumor size in a tumor-bearing mouse, where the cell permeable RUNX3 recombinant protein (HM₃R3) according to the present invention was administered via subcutaneous injection for 21 days, as compared with a control mouse.

FIG. 15 is a photograph of immunohistochemical staining showing the inhibitory effect on cell cycle and metastasis in mouse lung and tumor tissues extracted from a mouse administered with the cell permeable RUNX3 recombinant protein (HM₃R3) according to the present invention.

FIG. 16 is a photograph of a terminal deoxynucleotidyl transferase-mediated dUTP nick-end labeling (TUNEL) analysis showing the apoptosis-inducing effect in a mouse tumor tissue extracted from a mouse administered with the cell permeable RUNX3 recombinant proteins (HM₂R3 and HM₃R3) according to the present invention.

FIG. 17 is a photograph of an ApopTag analysis showing the apoptosis-inducing effect in a mouse tumor tissue extracted from a mouse administered with each of the cell permeable RUNX3 recombinant proteins (HM₂R3 and HM₃R3) via subcutaneous injection.

FIG. 18 is a photograph of a microarray analysis showing differential gene expression in a mouse tumor tissue extracted from a mouse administered with the cell permeable RUNX3 recombinant protein (HM₃R3) according to the present invention.

### BEST MODE FOR CARRYING OUT THE INVENTION

The present invention provides cell permeable RUNX3 recombinant proteins (CP-RUNX3) capable of mediating the transport of a tumor and metastasis suppressor RUNX3 into a cell in which the tumor and metastasis suppressor RUNX3 is fused to a macromolecule transduction domain (MTD) and, thereby, imparted with cell permeability; and polynucleotides encoding each of the cell permeable RUNX3 recombinant proteins.

The present invention is characterized in that a tumor and metastasis suppressor RUNX3 which is a macromolecule incapable of being introduced into a cell is fused to a specific macromolecule transduction domain (hereinafter, “MTD”) peptide so as to provide cell permeability, and thus, can be effectively transported into a cell. The MTD peptide may be fused to the N-terminus, the C-terminus, or both termini of the tumor and metastasis suppressor RUNX3.

The present invention has developed cell permeable RUNX3 recombinant proteins that are engineered by fusing a tumor and metastasis suppressor RUNX3 to one of five MTD domains capable of mediating the transport of a macromolecule into a cell.

The term “cell permeable RUNX3 recombinant protein” as used herein refers to a covalent bond complex bearing a MTD and a tumor and metastasis suppressor protein RUNX3, where they are functionally linked by genetic fusion or chemical coupling. Here, the term “genetic fusion” refers to a co-linear, covalent linkage of two or more proteins or fragments thereof via their individual peptide backbones, through genetic expression of a polynucleotide molecule encoding those proteins.

RUNX3 is a tumor and metastasis suppressor protein that activates p21, which inhibits the cell cycle and induces apoptosis, and suppresses VEGF which induces metastasis. RUNX3 has an amino acid sequence represented by SEQ ID NO: 2, while a polynucleotide encoding the same has a nucleotide sequence represented by SEQ ID NO: 1. RUNX3 functions as an important target protein in the TGF-β signal transduction pathway.

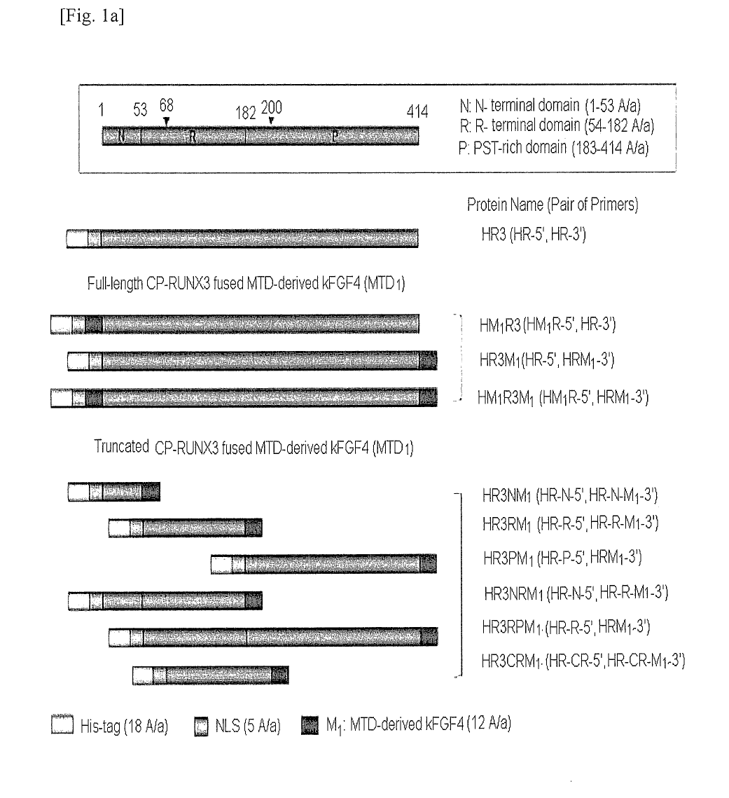

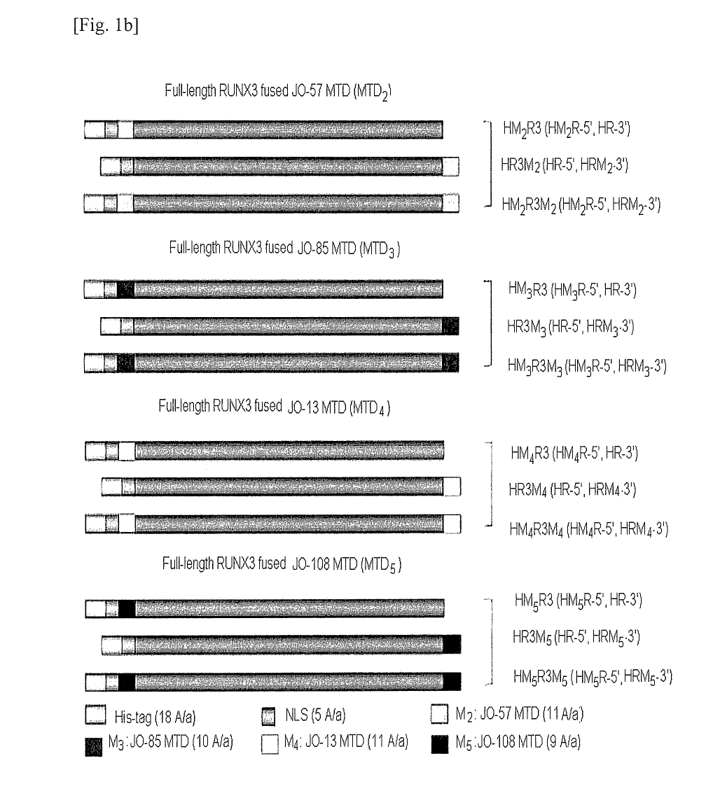

The amino acid sequence of the tumor and metastasis suppressor RUNX3, i.e., SEQ ID NO: 2, is composed of a N-terminal domain corresponding to amino acid residues 1-53, a R-terminal domain corresponding to amino acid residues 54-182, and a PST-rich domain corresponding to amino acid residues 183-414 (see FIG. 1a).

For the MTD capable of being fused to the tumor and metastasis suppressor

RUNX3, cell permeable peptides having an amino acid sequence selected from the group consisting of SEQ ID NOS: 3 to 196 may be used. The MTD having one of the amino acid sequences represented by SEQ ID NOS: 3 to 196 is a cell permeable polypeptide which is capable of mediating the transport of a biologically active molecule, such as a polypeptide, a protein domain, or a full-length protein across the cell membrane. Suitable MTDs for the present invention include a hydrophobic region showing cell membrane targeting activity by forming a helix structure at a signal peptide which is composed of an N-terminal domain, a hydrophobic domain and a C-terminal domain containing a secreted protein cleavage site. These MTDs can directly penetrate the cell membrane without causing any cell damage, transport a target protein into a cell, and thus, allow the target protein to exhibit its desired function.

The MTDs having the amino acid sequences represented by SEQ ID NOS: 3 to 196 and capable of being fused to a tumor and metastasis suppressor RUNX3 according to the present invention are summarized in the following Tables 1a to 1i.

TABLE 1a

| | | | SEQ |
|---|---|---|---|
| | | | ID |
| MTD | Origin | Amino acid sequence | NO |
| kFGF4- | kaposi fibroblast growth factor 4, kFGF4 | Ala Ala Val Leu Leu Pro Val Leu | 3 |
| derived | | Leu Ala Ala Pro | |
| MTD | | | |
| JO-01 | CAC04038 putative NLP/P60-family | Ala Val Val Val Cys Ala Ile Val | 4 |
| | secreted protein [Streptomyces | Leu Ala Ala Pro | |
| | coelicolorA3(2)] | | |
| JO-02 | NP_057021 phosphatidylinositol glycan, | Pro Leu Ala Leu Leu Val Leu Leu | 5 |
| | class T precursor [Homo sapiens] | Leu Leu Gly Pro | |
| JO-03 | NP_072171 chorionic | Leu Leu Leu Ala Phe Ala Leu Leu | 6 |
| | somatomammotropin hormone 2 isoform | Cys Leu Pro | |
| | 3 [Homo sapiens] | | |
| JO-04 | NP_932156 nudix -type motif 9 isoform a | Leu Leu Gly Ala Leu Ala Ala Val | 7 |
| | [Homo sapiens] | Leu Leu Ala Leu Ala | |
| JO-05 | NP_057327 NAD(P)H:quinone | Pro Val Leu Leu Ala Leu Gly Val | 8 |
| | oxidoreductase type 3, polypeptide A2 | Gly Leu Val Leu Leu Gly Leu Ala | |
| | [Homo sapiens] | | |
| JO-06 | CAD55300 putative secreted protein. | Ala Ala Ala Ala Val Leu Leu Ala | 9 |
| | [Streptomyces coelicolor A3(2)] | Ala | |
| JO-07 | NP_629514 secreted protein | Ile Val Val Ala Val Val Val Ile | 10 |
| | [Streptomyces coelicolor A3(2)] | | |
| JO-08 | CAB57190 putative secreted chitin | Ala Val Leu Ala Pro ValVal Ala | 11 |
| | binding protein [Streptomyces coelicolor | Val | |
| | A3(2)] | | |
| JO-09 | CAB51015 putative secreted protein | Leu Ala Val Cys Gly Leu Pro Val | 12 |
| | [Streptomyces coelicolor A3(2)] | Val Ala Leu Leu Ala | |
| JO-10 | NP_625021 glycosyl hydrolase (secreted | Leu Gly Gly Ala Val Val Ala Ala | 13 |
| | protein) [Streptomyces coelicolor A3(2)] | Pro Val Ala Ala Ala Val Ala Pro | |
| JO-11 | NP_630686 secreted protein | Leu Leu Leu Val Leu Ala Val Leu | 14 |
| | [Streptomyces coelicolor A3(2)] | Leu Ala Val Leu Pro | |
| JO-12 | NP_057329 dehydrogenase/reductase | Leu Leu Ile Leu Leu Leu Leu Pro | 15 |
| | (SDR family) member 8 [Homo sapiens] | Leu Leu Ile Val | |
| JO-13 | NP_639877 putative secreted protein | Leu Ala Ala Ala Ala Leu Ala Val | 16 |
| | [Streptomyces coelicolor A3(2)] | Leu Pro Leu | |
| JO-14 | NP_699201 protease inhibitor 16 | Phe Leu Met Leu Leu Leu Pro Leu | 17 |
| | precursor [Homo sapiens] | Leu Leu Leu Leu Val Ala | |
| JO-15 | NP_639871 putative secreted protein | Ala Ala Ala Ala Ala Ala Leu Gly | 18 |
| | [Streptomyces coelicolor A3(2)] | Leu Ala Ala Ala Val Pro Ala | |
| JO-16 | CAB85250 putative secreted protein | Leu Leu Leu Ala Ala Leu Leu Leu | 19 |
| | [Neisseria meningitidis Z2491] | Ile Ala Phe Ala Ala Val | |
| JO-17 | NP_626397 small secreted hydrophilic | Ala Leu Ala Ala Val Val Leu Ile | 20 |
| | protein [Streptomyces coelicolor A3(2)] | Pro Leu Gly Ile Ala Ala | |
| JO-18 | CAB38593 putative secreted protein | Ala Ala Leu Ala Leu Gly Val Ala | 21 |
| | [Streptomyces coelicolor A3(2)] | Ala Ala Pro Ala Ala Ala Pro Ala | |
| JO-19 | CAB57190 putative secreted chitin | Ala Ala Leu Ile Gly Ala Val Leu | 22 |
| | binding protein [Streptomyces coelicolor | Ala Pro Val Val Ala Val | |
| | A3(2)] | | |
| JO-20 | NP_626007 secreted cellulose-binding | Ala Ala Gly Ile Ala Val Ala Ile Ala | 23 |
| | protein [Streptomyces coelicolor A3(2)] | Ala Ile Val Pro Leu Ala | |
| JO-21 | NP_625632 secreted protein | Ile Ala Val Ala Ile Ala Ala Ile Val | 24 |
| | [Streptomyces coelicolor A3(2)] | Pro Leu Ala | |

TABLE 1b

| | | | SEQ ID |
|---|---|---|---|
| MTD | Origin | Amino acid sequence | NO |
| JO-22 | CAC31790 putative secreted protein | Val Ala Met Ala Ala Ala Ala Val | 25 |
| | [Mycobacterium leprae] | Leu Ala Ala Pro Ala Leu Ala | |
| JO-23 | NP_630266 secreted protein | Leu Ala Val Leu Val Leu Leu Val | 26 |
| | [StrePtomyces coelicolor A3(2)] | Leu Leu Pro | |
| JO-24 | NP_630165 secreted protein | Val Val Ala Val Leu Ala Pro Val | 27 |
| | [StrePtomyces coelicolor A3(2)] | Leu | |
| JO-25 | NC_003888 secreted protein | Ala Ala Leu Leu Leu Pro Leu Leu | 28 |
| | [StrePtomyces coelicolor A3(2)] | Leu Leu Leu Pro | |
| JO-26 | NP_627363 secreted protein | Pro Ala Ala Val Ala Ala Leu Leu | 29 |
| | [StrePtomyces coelicolor A3(2)] | Val Ile | |
| JO-27 | NP_631288 secreted protein | Leu Leu Ile Ala Ala Leu Leu Pro | 30 |
| | [StrePtomyces coelicolor A3(2)] | | |
| JO-28 | NP_630325 secreted protein | Ala Ala Val Val Leu Leu Pro Leu | 31 |
| | [StrePtomyces coelicolor A3(2)] | Ala Ala Ala Pro | |
| JO-29 | NP_631289 secreted protein | Ala Ala Ala Ala Ala Ala Leu Leu | 32 |
| | [StrePtomyces coelicolor A3(2)] | Val Pro | |
| JO-30 | CAB51015 Putative secreted protein | Leu Pro Val Val Ala Leu Leu Ala | 33 |
| | [StrePtomyces coelicolor A3(2)] | | |
| JO-31 | NP_629515chitinase C ( secreted protein) | Ala Ala Ala Leu Ala Ala Pro Leu | 34 |
| | [StrePtomyces coelicolor A3(2)] | Ala Leu Pro | |
| JO-32 | NP_940995 Clq and tumor necrosis | Leu Leu Leu Ala Leu Leu Leu Ala | 35 |
| | factor related Protein 1 isoform 1 | Ala | |
| | [Homo saPiens] | | |
| JO-33 | NP_854150 POSSIBLE CONSERVED | Ala Val Ala Val Val Ala Leu Leu | 36 |
| | SECRETED PROTEIN [Mycobacterium | | |
| | bovis AF2122/97] | | |
| JO-34 | NP_630361 Probable secreted protein | Leu Leu Leu Ile Ile Val Leu Leu | 37 |
| | [StrePtomyces coelicolor A3(2)] | Ile Val Pro | |
| JO-35 | P39790 Extracellular metalloProtease | Leu Ala Leu Ala Ala Ala Val Val | 38 |
| | Precursor | Pro | |
| JO-36 | CAA19252 Putative liPoProtein | Pro Ala Ala Leu Ala Leu Leu Leu | 39 |
| | [StrePtomyces coelicolor A3(2)] | Val Ala | |
| JO-37 | P_625685 large secreted protein | Ile Val Ala Leu Leu Leu Val Pro | 40 |
| | [StrePtomyces coelicolor A3(2)] | Leu Val Leu Ala Ile Ala Ala Val | |
| | | Leu | |
| JO-38 | NP_625685 large secreted protein | Ile Val Ala Leu Leu Leu Val Pro | 41 |
| | [StrePtomyces coelicolor A3(2)] | | |
| JO-39 | NP_625685 large secreted protein | Pro Leu Val Leu Ala Ile Ala Ala | 42 |
| | [StrePtomyces coelicolor A3(2)] | Val Leu | |
| JO-40 | NP_808800 golgi PhosPhoProtein 2 | Pro Leu Val Leu Ala Ala Leu Val | 43 |
| | [Homo saPiens] | Ala | |
| JO-41 | NP_626993 secreted protein | Ala Ala Ala Leu Leu Ala Val Ala | 44 |
| | [StrePtomyces coelicolor A3(2)] | | |
| JO-42 | NP_004863 thymic dendritic cell-derived | Pro Leu Leu Leu Leu Ala Leu Ala | 45 |
| | factor 1 [Homo saPiens] | | |
| JO-43 | NP_631398 secreted protein | Ala Leu Ala Leu Val Val Ala | 46 |
| | [StrePtomyces coelicolor A3(2)] | | |
| JO-44 | NP_627373 Penicillin-binding Protein | Val Ala Ala Val Val Val Ala Ala | 47 |
| | (seceted protein) [StrePtomyces | | |
| | coelicolor A3(2)] | | |

TABLE 1c

| | | | SEQ ID |
|---|---|---|---|
| MTD | Origin | Amino acid sequence | NO |
| JO-45 | NP056226 sulfatase modifying factor 2 | Pro Leu Leu Pro Leu Leu Leu Leu | 48 |
| | [Homo saPiens] | Val | |
| JO-46 | NP54998 Conserved hypothelial | Val Val Leu Val Val Val Leu Pro | 49 |
| | secreted protein [Mycobacterium bovis | Leu Ala Val Leu Ala | |
| | AF2122/97] | | |
| JO-47 | NP627512 secreted protein | Ala Ala Ala Val Pro Val Leu Val | 50 |
| | [StrePtomyces coelicolor A3(2)] | Ala Ala | |
| JO-48 | NP110448 phospholipase A2, group | Pro Ala Leu Leu Leu Leu Leu Leu | 51 |
| | XIIA [Homo sapiens] | Ala Ala Val Val | |
| JO-49 | NP003245 tissue inhibitor of | Pro Leu Ala Ile Leu Leu Leu Leu | 52 |
| | metalloproteinase 1 precursor [Homo | Leu Ile Ala Pro | |
| | sapiens] | | |
| JO-50 | NP002978 small inducible cytokine A17 | Pro Leu Leu Ala Leu Val Leu Leu | 53 |
| | precursor [Homo sapiens] | Leu Ala Leu Ile Ala | |
| JO-51 | NP001012495 stromal cell derived factor | Val Val Val Ala Val Leu Ala Leu | 54 |
| | 1 isoform gamma precursor [Mus | Val Leu Ala Ala Leu | |
| | musculus] | | |
| JO-52 | NP775628 ficolin 3 isoform 2 precursor | Pro Leu Leu Leu LeuLeu Pro Ala | 55 |
| | [Homo sapiens] | Leu | |
| JO-53 | NP624483 secreted protein | Leu Ala Ala Val Ala Ala Leu Ala | 56 |
| | [treptomyces coelicolor A3(2)] | Val Val Val Pro | |
| JO-54 | NP997465 HERV-FRD provirus | Leu Leu Leu Leu Val Leu Ile Leu | 57 |
| | ancestral Env polyprotein sapiens] | Pro Leu Ala Ala | |
| JO-55 | NP854234 posible conserved secreted | Leu Ala Val Val Val Val Ala Ala | 58 |
| | protein [Mycobacterium bovis | Val | |
| | AF2122/97] | | |
| JO-56 | P23284 Peptidyl-prolyl cis-trans | Val Leu Leu Ala Ala Ala Leu Ile | 59 |
| | isomerase B precursor (PPIase) | Ala | |
| | (Rotamase) (Cyclophilin B) | | |
| JO-57 | CAD05047 hypothetical secreted protein | Leu Ile Ala Leu Leu Ala Ala Pro | 60 |
| | [Salmonella enterica subsp. Enterica | Leu Ala | |
| | serovar Typhi] | | |
| JO-58 | P05067Amyloid beta A4 protein precursor | Leu Ala Leu Leu Leu Leu Ala Ala | 61 |
| | (APP) (ABPP) (Alzheimer disease | | |
| | amyloid protein) | | |
| JO-59 | NP004878 small inducible cytokine B14 | Leu Leu Ala Ala Ala Leu Leu Leu | 62 |
| | precursor [Homo sapiens] | Leu Leu Leu Ala | |
| JO-60 | NP626589 secreted protein | Val Ile Ile Ala Leu Ile Val Ile | 63 |
| | [Streptomyces coelicolor A3(2)] | Val Ala | |
| JO-61 | NP626589 secreted protein | Val Val Leu Val Val Ala Ala Val | 64 |
| | [Streptomyces coelicolor A3(2)] | Leu Ala Leu | |
| JO-62 | NP856548 SOLUBLE SECRETED | Val Ala Val Ala Ile Ala Val Val | 65 |
| | ANTIGEN MPB53 [Mycobacterium bovis | Leu | |
| | AF2122/97] | | |
| JO-63 | NP 629854 secreted protein | Pro Leu Ile Val Val Val Ala Ala | 66 |
| | [Streptomyces coelicolor A3(2)] | Ala Val Val Ala Val | |
| JO-64 | AAB59058 lambda receptor protein | Pro Leu Ala Val Ala Val Ala Ala | 67 |
| | [Escherichia coli] | Val Ala Ala | |
| JO-65 | NP_825185 NLP/P60-family secreted | Ala Ala Ile Ala Leu Val Ala Val | 68 |
| | protein [Streptomyces avermitilis MA- | Val Leu | |
| | 4680] | | |

TABLE 1d

| MTD | Origin | Amino acid sequence | [symbol] |
|---|---|---|---|
| JO-66 | NP_626568 secreted protein | Ala Ala Ala Leu Ala Ala Ile Ala | 69 |
| | [Streptomyces coelicolor A3(2)] | Val Ile | |
| JO-67 | NP_626568 secreted protein | Ala Ala Ala Pro Ala Val Ala Ala | 70 |
| | [Streptomyces coelicolor A3(2)] | | |
| JO-68 | NP_625639 secreted protein | Leu Leu Leu Ala Ala Leu Pro | 71 |
| | [Streptomyces coelicolor A3(2)] | | |
| JO-69 | CAC32053 putative secreted protein | Ala Leu Leu Ala Val Val Ala Ala | 72 |
| | [Mycobacterium leprae] | | |
| JO-70 | NP_630954 secreted protein | Ala Val Val Val Val Leu Pro Ile | 73 |
| | [Streptomyces coelicolor A3(2)] | Leu Leu | |
| JO-71 | P97300 Neuroplastin precursor (Stromal | Ala Leu Ala Leu Leu Leu Leu Val | 74 |
| | cell-derived receptor 1) (SDR-1) | Pro | |
| JO-72 | AAA41949 Rat parotid gland acidic | Leu Val Val Leu Leu Ala Ala Leu | 75 |
| | proline-rich protein mRNA, complete | Leu Val Leu | |
| | CDS | | |
| JO-73 | AAA17887 Drosophila melanogaster | Pro Val Leu Leu Leu Leu Ala Pro | 76 |
| | spatzle (spz) gene | | |
| JO-74 | NP_627867 conserved secreted protein | Ala Leu Ala Val Val Ala Ala Pro | 77 |
| | [Streptomyces coelicolor A3(2)] | | |
| JO-75 | NP_631283 secreted protein | Val Ile Val Ala Leu Leu Ala Val | 78 |
| | [Streptomyces coelicolor A3(2)] | | |
| JO-76 | NP_003231 endometrial bleeding | Ala Leu Val Leu Pro Leu Ala Pro | 79 |
| | associated factor preproprotein [Homo | | |
| | sapiens] | | |
| JO-77 | CAB76313 putative secreted protein | Ala Val Ala Leu Leu Ile Leu Ala | 80 |
| | [Streptomyces coelicolor A3(2)] | Val | |
| JO-78 | P07198 Xenopsin precursor [Contains: | Val Leu Leu Ala Val Ile Pro | 81 |
| | Xenopsin precursor fragment (XPF); | | |
| | Xenopsin] | | |
| JO-79 | NP_631293 secreted protein | Leu Ile Val Ala Ala Val Val Val | 82 |
| | [Streptomyces coelicolor A3(2)] | Val Ala Val Leu Ile | |
| JO-80 | NP_626373 secreted protein | Ala Val Val Val Ala Ala Pro | 83 |
| | [Streptomyces coelicolor A3(2)] | | |
| JO-81 | NP_624952 secreted cellulose-binding | Leu Ala Ala Val Leu Leu Leu Ile | 84 |
| | protein [Streptomyces coelicolor A3(2)] | Pro | |
| JO-82 | NP_009104 protease, serine, 23 precursor | Leu Leu Leu Leu Leu Leu Ala Val | 85 |
| | [Homo sapiens] | Val Pro | |
| JO-83 | AAK63068 phytotoxic protein PcF | Ala Val Ala Leu Val Ala Val Val | 86 |
| | precursor [Phytophthora cactorum] | Ala Val Ala | |
| JO-84 | NC_003903 Streptomyces coelicolor | Leu Val Ala Ala Leu Leu Ala Val | 87 |
| | A3(2) plasmid SCP1, complete sequence. | Leu | |
| JO-85 | NP_629842 peptide transport system | Leu Leu Ala Ala Ala Ala Ala Leu | 88 |
| | secreted peptide binding protein | Leu Leu Ala | |
| | [Streptomyces coelicolor A3(2)] | | |
| JO-86 | NP_854067 Possible secreted protein | Leu Ala Val Leu Ala Ala Ala Pro | 89 |
| | [Mycobacterium bovis AF2122/97] | | |
| JO-87 | NP_627802 secreted protein | Val Val Val Leu Leu Val Leu Leu | 90 |
| | [Streptomyces coelicolor A3(2)] | Ala Leu Val Val Val | |
| JO-88 | NP_627802 secreted protein | Val Val Ile Ala Val Val Pro | 91 |
| | [Streptomyces coelicolor A3(2)] | | |

TABLE 1e

| | | | SEQ ID |
|---|---|---|---|
| MTD | Origin | Amino acid sequence | NO |
| JO-89 | NP_624483 secreted protein | Leu Ala Ala Val Ala Ala Leu Ala | 92 |
| | [Streptomyces coelicolor A3(2)] | Val Val | |
| JO-90 | NP_627802 secreted protein | Val Leu Leu Val Leu Leu Ala Leu | 93 |
| | [Streptomyces coelicolor A3(2)] | Val | |
| JO-91 | NP_625203 secreted protein | Pro Val Leu Val Pro Ala Val Pro | 94 |
| | [Streptomyces coelicolor A3(2)] | | |
| JO-92 | NP_630960 secreted protein | Pro Ala Leu Ala Leu Ala Leu Ala | 95 |
| | [Streptomyces coelicolor A3(2)] | | |
| JO-93 | NP_630670 secreted protein | Ala Ala Ala Ala Pro Ala Leu Ala | 96 |
| | [Streptomyces coelicolor A3(2)] | | |
| JO-94 | NP_630493 secreted protein | Ile Val Leu Pro Val Leu Ala Ala | 97 |
| | [Streptomyces coelicolor A3(2)] | Pro | |
| JO-95 | CAC29994 putative secreted protein | Leu Val Leu Leu Leu Leu Pro Leu | 98 |
| | [Mycobacterium leprae] | Leu Ile | |
| JO-96 | NP_624483 secreted protein | Leu Ala Ala Val Ala Pro Ala Leu | 99 |
| | [Streptomyces coelicolor A3(2)] | Ala Val Val | |
| JO-97 | NP_037375 secretogranin III | Ile Leu Val Leu Val Leu Pro Ile | 100 |
| | [Homo sapiens] | | |
| JO-98 | NP_009199 V-set and immunoglobulin | Ile Leu Leu Pro Leu Leu Leu Leu | 101 |
| | domain containing 4 [Homo sapiens] | Pro | |
| JO-99 | NP_733650 secreted hydrolase | Ile Ala Pro Ala Val Val Ala Ala | 102 |
| | [Streptomyces coelicolor A3(2)] | Leu Pro | |
| JO-100 | NP_057540 transmembrane protein 9 | Leu Leu Leu Val Ala Val Val Pro | 103 |
| | [Homo sapiens] | Leu Leu Val Pro | |
| JO-101 | CAI74362 hypothetical protein | Leu Ile Leu Leu Leu Leu Pro Ile | 104 |
| | [Theileria annulata] | Ile | |
| JO-102 | NP_630671 secreted protein | Ala Val Leu Ala Ala Pro Ala Val | 105 |
| | [Streptomyces coelicolor A3(2)] | Leu Val | |
| JO-103 | NP_065695 TMEM9 domain family, | Leu Ala Leu Pro Val Leu Leu Leu | 106 |
| | member B [Homo sapiens] | Ala | |
| JO-104 | P06908 Pulmonary surfactant-associated | Leu Ala Leu Ala Leu Leu Leu | 107 |
| | protein A precursor (SP-A) (PSP-A) | | |
| | (PSAP). | | |
| JO-105 | NP_639721 putative secreted protein | Val Ala Val Pro Leu Leu Val Val | 108 |
| | [Streptomyces coelicolor A3(2)] | Ala | |
| JO-106 | NP_854954 CONSERVED PROBABLE | Ala Val Ala Val Ala Pro Val Ala | 109 |
| | SECRETED PROTEIN [Mycobacterium | Ala Ala Ala | |
| | bovis AF2122/97] | | |
| JO-107 | NP_627759 secreted protein | Ala Ala Ala Val Val Ala Ala Val | 110 |
| | [Streptomyces coelicolor A3(2)] | Pro Ala Ala | |
| JO-108 | NP_003842 cellular repressor of E1A- | Ala Leu Leu Ala Ala Leu Leu Ala | 111 |
| | stimulated genes [Homo sapiens] | Pro | |
| JO-109 | NP_003842 cellular repressor of E1A- | Leu Leu Ala Leu Leu Val Pro | 112 |
| | stimulated genes [Homo sapiens] | | |
| JO-110 | NP_003842 cellular repressor of E1A- | Ala Leu Leu Ala Ala Leu Leu Ala | 113 |
| | stimulated genes [Homo sapiens] | Leu Leu Ala Leu Leu Val | |
| JO-111 | NP_000589 Homo sapiens insulin-like | Ala Ala Ala Leu Pro Leu Leu Val | 114 |
| | growth factor binding protein 3 (IGFBP3), | Leu Leu Pro | |

TABLE 1f

| | | | SEQ ID |
|---|---|---|---|
| MTD | Origin | Amino acid sequence | NO |
| JO-112 | CAB59459 putative secreted protein | Ala Ala Ala Val Pro Ala Ala Leu | 115 |
| | [Streptomyces coelicolor A3(2)] | Ala Pro | |
| JO-113 | NP_628917 secreted protein | Ala Ala Leu Ala Val Ala Ala Leu | 116 |
| | [Streptomyces coelicolor A3(2)] | Ala Ala | |
| JO-114 | NP_624695 secreted protein | Ala Val Leu Ala Ala Ala Val Pro | 117 |
| | [Streptomyces coelicolor A3(2)] | | |
| JO-115 | NP_624695 secreted protein | Val Ala Ala Leu Pro Ala Pro Ala | 118 |
| | [Streptomyces coelicolor A3(2)] | | |
| JO-116 | NP_624791 secreted protein | AlaLeu Ala Leu Ala Val Pro Ala | 119 |
| | [Streptomyces coelicolor A3(2)] | Val Leu Pro | |
| JO-117 | CAB45579 putative secreted protein | Ala Ala Leu Leu Pro Ala Ala Val | 120 |
| | [Streptomyces coelicolor A3(2)] | Ala Val Pro | |
| JO-118 | NP_627066 secreted protein | Ala Val Val Val Ala Leu Ala Pro | 121 |
| | [Streptomyces coelicolor A3(2)] | | |
| JO-119 | NP_630174 secreted substrate-binding | Ala Ala Ala Val Ala Leu Pro Ala | 122 |
| | protein [Streptomyces coelicolor A3(2)] | Ala Ala Ala Leu Leu Ala | |
| JO-120 | P06727 Apolipoprotein A-IV precursor | Ala Val Val Leu Pro Leu Ala Leu | 123 |
| | (Apo-AIV) (ApoA-IV) Homo sapiens | Val Ala Val Ala Pro | |
| JO-121 | Q62087 Serum paraoxonase/lactonase 3. | Leu Val Ala Leu Pro Leu Leu Pro | 124 |
| | Mus musculus | | |
| JO-122 | NP_627123 probable secreted penicillin- | Val Val Val Pro Leu Leu Leu Ile | |
| | binding protein[Streptomyces coelicolor | Val Pro | 125 |
| | A3(2)] | | |
| JO-123 | CAC30224 putative secreted hydrolase | Leu Ala Val Val Leu Ala Val Pro | 126 |
| | [Mycobacterium leprae] | | |
| JO-124 | OZZQAM circumsporozoite protein | Leu Leu Ala Val Pro Ile Leu Leu | 127 |
| | precursor - Plasmodium cynomolgi | Val Pro | |
| JO-125 | Q15166 Serum paraoxonase/lactonase 3. | Leu Val Ala Leu Val Leu Leu Pro | 128 |
| | Homo sapiens | | |
| JO-126 | NP_060220 all-trans-13,14-dihydroretinol | Leu Val Leu Leu Leu Ala Val Leu | 129 |
| | saturase [Homo sapiens] | Leu Leu Ala Val Leu Pro | |
| JO-127 | AL627273 Salmonella enterica serovar | Leu Leu Ala Pro Val Val Ala Leu | 130 |
| | Typhi (Salmonella typhi) strain CT18, | Val Ile Leu Pro | |
| JO-128 | NP_625987 secreted protein | Val Leu Ala Val Leu Ala Val Pro | 131 |
| | [Streptomyces coelicolor A3(2)] | Val Leu Leu Leu Pro | |
| JO-129 | CAB45474 putative secreted protein | Val Val Ile Ala Val Val Pro Val | 132 |
| | [Streptomyces coelicolor A3(2)] | Val Val | |
| JO-130 | CAB45474 putative secreted protein | Leu Leu Val Leu Leu Ala Leu Val | 133 |
| | [Streptomyces coelicolor A3(2)] | Val Val Pro | |
| JO-131 | CAB36605 putative secreted protein | Val Leu Leu Ala Leu Pro Val Val | 134 |
| | [Streptomyces coelicolor A3(2)] | Ala Ala Pro | |
| JO-132 | NP_7628377NLP/P60-family secreted | Ala Val Val Val Pro Ala Ile Val | 135 |
| | protein [Streptomyces coelicolor A3(2)] | Leu Ala Ala Pro | |

TABLE 1g

| | | | SEQ ID |
|---|---|---|---|
| MTD | Origin | Amino acid sequence | NO |
| JO-133 | CAB59594 putative secreted protein | Ala Val Leu Val Pro Ala Ala Ala | 136 |
| | [Streptomyces coelicolor A3(2)] | Leu Val Pro | |
| JO-134 | NP_624974 secreted protein | Val Val Ala Ala Leu Pro Leu Val | 137 |
| | [Streptomyces coelicolor A3(2)] | Leu Pro | |
| JO-135 | NP_733682 secreted ATP/GTP binding | Ala Ala Val Ala Leu Pro Ala Ala | 138 |
| | protein [Streptomyces coelicolor A3(2)] | Ala Pro | |
| JO-136 | P27169 Serum paraoxonase/arylesterase 1 | | |
| | (PON 1) (Serum aryldialkylphosphatase | Leu Ile Ala Leu Pro Leu Leu Pro | 139 |
| | 1) (A-esterase 1) Homo sapiens | | |
| JO-137 | P52430 Serum paraoxonase/arylesterase 1 | Leu Leu Ala Leu Pro Leu Val Leu | 140 |
| | (PON 1) (Serum aryldialkylphosphatase | Val Leu Ala Leu Pro | |
| | 1) (A-esterase 1) Homo sapiens | | |
| JO-138 | NP_626569 secreted protein | Ile Val Pro Leu Leu Leu Ala Ala | 141 |
| | [Streptomyces coelicolor A3(2)] | Pro | |
| JO-139 | NP_940995 Clq and tumor necrosis factor | Leu Leu Leu Ala Pro Leu Leu Leu | 142 |
| | related protein 1 isoform 1 [Homo | Ala Pro | |
| | sapiens] | | |
| JO-140 | NP_626174 large secreted protein | Leu Ala Ala Leu Pro Val Ala Ala | 143 |
| | [Streptomyces coelicolor A3(2)] | Val Pro | |
| JO-141 | CAB83860 putative protein-export | Ala Leu Ala Val Ile Val Leu Val | 144 |
| | integral membrane protein [Neisseria | Leu Leu | |
| | meningitidis Z2491] | | |
| JO142 | NP_001009551 cornichon-like isoform 2 | Leu Ala Leu Leu Leu Pro Ala Ala | 145 |
| | [Homo sapiens] | Leu Ile | |
| JO-143 | NP_626808 secreted protein | Ala Leu Leu Pro Leu Leu Ala Val | 146 |
| | [Streptomyces coelicolor A3(2)] | Val Leu Pro | |
| JO-144 | NP_639798 putative secreted protein | Ala Ile Ala Val Pro Val Leu Ala | 147 |
| | [Streptomyces coelicolor A3(2)] | Ala Pro | |
| JO-145 | NP_000492 Homo sapiens elastin | Ala Ala Ala Pro Val Leu Leu Leu | 148 |
| | (supravalvular aortic stenosis) | Leu Leu | |
| JO-146 | NP_630680 secreted sugar binding protein | Ala Ala Ala Val Ala Val Leu Ala | 149 |
| | [Streptomyces coelicolor A3(2)] | Leu Ala Pro | |
| JO-147 | CAB56129 putative secreted protein | Ala Ala Leu Ala Ala Leu Val Val | 150 |
| | [Streptomyces coelicolor A3(2)] | Ala Ala Pro | |
| JO-148 | NP_625109 secreted solute-binding | Ala Ala Leu Ala Ala Val Pro Leu | 151 |
| | lipoprotein [Streptomyces coelicolor | Ala Leu Ala Pro | |
| | A3(2)] | | |
| JO-149 | NP_733579 secreted sugar-binding | Ala Leu Ala Val Ala Ala Pro Ala | 152 |
| | protein [Streptomyces coelicolor A3(2)] | Leu Ala Leu Leu Pro | |
| JO-150 | NP_630126 secreted chitinase ( secreted | Ala Ala Leu Pro Ala Ala Ala Pro | 153 |
| | protein) [Streptomyces coelicolor | | |
| | A3(2)] | | |
| JO-151 | NP_630126 secreted chitinase (secreted | Ala Ala Ala Pro Val Ala Ala Val | 154 |
| | protein) [Streptomyces coelicolor | Pro | |
| | A3(2)] | | |
| JO-152 | NP_872425 secretory protein LOC348174 | Leu Leu Ala Val Leu Leu Ala Leu | 155 |
| | [Homo sapiens] | Leu Pro | |
| JO-153 | NP_630107 secreted protein | Val Leu Ala Leu Leu Val Ala Val | 156 |
| | [Streptomyces coelicolor A3(2)] | Val Pro | |
| JO-154 | NP_733688 peptide-binding transport | Ala Leu Val Val Pro Ala Ala Val | 157 |
| | protein [Streptomyces coelicolor A3(2)] | Pro | |

TABLE 1h

| | | | SEQ ID |
|---|---|---|---|
| MTD | Origin | Amino acid sequence | NO |
| JO-155 | NP_629904 secreted protein | Ala Val Val Leu Pro Leu Leu Leu | 158 |
| | [Streptomyces coelicolor A3(2)] | Pro | |
| JO-156 | YP_177852 MCE-FAMILY PROTEIN | Ala Val Ile Pro Val Ala Val Leu | 159 |
| | MCE3A [Mycobacterium tuberculosis | Val Pro | |
| | H37Rv] | | |
| JO-157 | CAA19627 putative secreted solute | Ala Ala Ala Val Pro Ala Ala Val | 160 |
| | binding protein [Streptomyces coelicolor | Leu Ala Pro | |
| | A3(2)] | | |
| JO-158 | NP_639884 putative large secreted protein | ValAla Val Pro Val Val Leu Ala | 161 |
| | [Streptomyces coelicolor A3(2)] | Ile Leu Pro | |
| JO-159 | P24327 Foldase protein prsA precursor. | Ile Ala Ile Ala Ala Ile Pro Ala | 162 |
| | | Ile Leu Ala Leu | |
| JO-160 | CAB84808 putative membrane | Ala Leu Ile Ala Pro Ala Leu Ala | 163 |
| | lipoprotein [Neisseria meningitidis Z2491] | AlaPro | |
| JO-161 | NP_639883 putative large secreted protein | Ala Ala Ile Ala Leu Val Ala Pro | 164 |
| | [Streptomyces coelicolor A3(2)] | Ala Leu | |
| JO-162 | NP_639883 putative large secreted protein | Leu Ala Pro Ala Val Ala Ala Ala | 165 |
| | [Streptomyces coelicolor A3(2)] | Pro | |
| JO-163 | NP_627362 secreted protein | Val Ala Ile Ile Val Pro Ala Val | 166 |
| | [Streptomyces coelicolor A3(2)] | Val Ala Ile Ala Leu Ile Ile | |
| JO-164 | NP_627362 secreted protein | Ala Val Val Ala Ile Ala Leu Ile | 167 |
| | [Streptomyces coelicolor A3(2)] | Ile | |
| JO-165 | NP_624625 secreted protein | Leu Ala Ala Val Pro Ala Ala Ala | 168 |
| | [Streptomyces coelicolor A3(2)] | Pro | |
| JO-166 | NP_624625 secreted protein | Ala Val Ala Ala Leu Pro Leu Ala | 169 |
| | [Streptomyces coelicolor A3(2)] | Ala Pro | |
| JO-167 | NP_624625 secreted protein | Leu Ala Ala Pro Ala Ala Ala Ala | 170 |
| | [Streptomyces coelicolor A3(2)] | Pro | |
| JO-168 | NP_626936 secreted protein | Leu Ala Ala Val Val Pro Val Ala | 171 |
| | [Streptomyces coelicolor A3(2)] | Ala Ala Val Pro | |
| JO-169 | NP_626936 secreted protein | Val Ala Ala Pro Ala Ala Ala Ala | 172 |
| | [Streptomyces coelicolor A3(2)] | Pro | |
| JO-170 | NP_626936 secreted protein | Ala Val Pro Val Pro Val Pro Leu | 173 |
| | [Streptomyces coelicolor A3(2)] | | |
| JO-171 | NP_085072 matrilin 2 isoform b precursor | Leu Leu Ile Leu Pro Ile Val Leu | 174 |
| | [Homo sapiens] | Leu Pro | |
| JO-172 | CAB94057 putative secreted protein | Ala Leu Ala Leu Pro Ala Leu Ala | 175 |
| | [Streptomyces coelicolor A3(2)] | Ile Ala Pro | |
| JO-173 | NP_624384 secreted protein | Ala Val Ile Pro Ile Leu Ala Val | 176 |
| | [Streptomyces coelicolor A3(2)] | Pro | |
| JO-174 | NP_733505 large, multifunctional | Leu Ile Leu Leu Leu Pro Ala Val | 177 |
| | secreted protein [Streptomyces coelicolor | Ala Leu Pro | |
| | A3(2)] | | |
| JO-175 | CAB45630 putative secreted protein | Ile Val Leu Ala Pro Val Pro Ala | 178 |
| | [Streptomyces coelicolor A3(2)] | Ala Ala | |
| JO-176 | NP_627887 secreted protein | Val Val Val Val Pro Val Leu Ala | 179 |
| | [Streptomyces coelicolor A3(2)] | Ala Ala Ala | |
| JO-177 | P06832 Bacillolysin precursor | Leu Val Ala Val Ala Ala Pro | 180 |

TABLE 1i

| | | | SEQ ID |
|---|---|---|---|
| MTD | Origin | Amino acid sequence | NO |
| JO-178 | NP_625998 secreted hydrolase | Leu Val Leu Ala Ala Pro Ala Ala | 181 |
| | [Streptomyces coelicolor A3(2)] | Leu Pro | |
| JO-179 | NP_625057 secreted protein | Leu Ile Ala Pro Ala Ala Ala Val | 182 |
| | [Streptomyces coelicolor A3(2)] | Pro | |
| JO-180 | NP_443750 ADP-ribosyltransferase 5 | Ala Leu Ala Ala Leu Pro Ile Ala | 183 |
| | precursor [Homo sapiens] | Leu Pro | |
| JO-181 | CAB84257 putative secreted protein | Ala Val Leu Leu Leu Pro Ala Ala | 184 |
| | [Neisseria meningitidis Z2491] | Ala | |
| JO-182 | P00634 Alkaline phosphatase precursor | Ile Ala Leu Ala Leu Leu Pro Leu | 185 |
| | (APase). | Leu | |
| JO-183 | NP_000933 peptidylprolyl isomerase B | Val Leu Leu Ala Ala Ala Leu Ile | 186 |
| | precursor [Homo sapiens] | Ala Pro | |
| JO-184 | CAB71258 putative secreted protein. | Ala Pro Ala Val Leu Pro Pro Val | 187 |
| | [Streptomyces coelicolor A3(2)] | Val Val Ile | |
| JO-185 | CAC31847 possible secreted protein | Val Val Gly Leu Leu Val Ala Ala | 188 |
| | [Mycobacterium leprae] | Leu | |
| JO-186 | NP_626948 secreted protein | Ala Ala Ile Ala Ala Ala Ala Pro | 189 |
| | [Streptomyces coelicolor A3(2)] | Leu Ala Ala | |
| JO-187 | NP_059120 cat eye syndrome critical | Leu Leu Leu Ala Val Ala Pro | 190 |
| | region protein 1 isoform a precursor | | |
| | [Homo sapiens] | | |
| JO-188 | NP_006519 tissue factor pathway | Leu Ile Leu Leu Leu Pro Leu Ala | 191 |
| | inhibitor [Homo sapiens] | Ala Leu | |
| JO-189 | P97299 Secreted frizzled-related protein 2 | Ala Leu Leu Leu Leu Val Leu Ala | 192 |
| | precursor (sFRP-2) (Secreted apoptosis- | | |
| | related protein 1) | | |
| JO-190 | NP_071447 tubulointerstitial nephritis | Leu Leu Leu Leu Leu Leu Pro Leu | 193 |
| | antigen-like 1 | Ala | |
| JO-191 | NP_056322 epidermal growth factor-like | Leu Ala Leu Pro Leu Leu Leu Pro | 194 |
| | protein 6 precursor [Homo sapiens] | | |
| JO-192 | NP_628035 secreted penicillin-binding | Leu Leu Val Leu Pro Leu Leu Ile | 195 |
| | protein [Streptomyces coelicolor A3(2)] | | |
| JO-193 | NP_683880 cathepsin H isoform b | Leu Pro Leu Leu Pro Ala Ala Leu | 196 |
| | precursor [Homo sapiens] | Val | |

In some embodiments, the present invention may employ a kaposi fibroblast growth factor 4 (kFGF4)-derived MTD having the amino acid sequence of SEQ ID NO: 3 (hereinafter, “MTD₁”), a JO-57 MTD having the amino acid sequence of SEQ ID NO: 60 which is a hypothetical protein derived from Salmonella enterica subsp. (hereinafter, “MTD₂”), a JO-85 MTD having the amino acid sequence of SEQ ID NO: 88 which is a peptide binding protein derived from Streptomyces coelicolor (hereinafter, “MTD₃”), a JO-13 MTD having the amino acid sequence of SEQ ID NO: 16 which is a putative secreted protein derived from Streptomyces coelicolor (hereinafter, “MTD₄”), and a JO-108 MTD having the amino acid sequence of SEQ ID NO: 111 which is a cellular repressor derived from Homo sapiens (hereinafter, “MTD₅”), as the MTD capable of mediating the transport of the tumor and metastasis suppressor RUNX3 into a cell.

The cell permeable RUNX3 recombinant proteins according to the present invention have a structure where one of the five MTDs (kFGF4-derived MTD: MTD₁, JO-57: MTD₂, JO-85: MTD₃, JO-13: MTD₄, JO-108: MTD₅) is fused to one terminus or both termini of a tumor and metastasis suppressor protein RUNX3, and a SV40 large T antigen-derived nuclear localization sequence (NLS) and a histidine-tag (His-Tag) affinity domain for easy purification are fused to one terminus of the resulting construct.

In another embodiment, the present invention relates to the construction of three full-length forms and six truncated forms of a cell permeable RUNX3 recombinant protein by using a kFGF4-derived MTD.

As used herein, the term “full-length form” refers to a construct including the entire N-terminal, R-terminal, and PST-rich domains of the tumor and metastasis suppressor protein RUNX3, while the term “truncated form” refers to a construct lacking any one or more of the N-terminal, R-terminal, and PST-rich domains thereof.

Referring to FIG. 1a, the full-length forms of the cell permeable RUNX3 recombinant protein are as follows:
1) HM₁R3, where a kFGF4-derived MTD is fused to the N-terminus of a full-length RUNX3,
2) HR3M₁, where a kFGF4-derived MTD is fused to the C-terminus of a full-length RUNX3, and
3) HM₁R3M₁ where a kFGF4-derived MTD is fused to both termini of a full-length RUNX3,
where a His-tag and a NLS derived from SV40 large T antigen are covalently coupled to the N-terminus of the above constructs.

As for the full-length forms of the cell permeable RUNX3 recombinant protein constructed by using a kFGF4-derived MTD as described above, HM₁R3 has an amino acid sequence represented by SEQ ID NO: 199, while a polynucleotide encoding the same has a nucleotide sequence represented by SEQ ID NO: 198; HR3M₁ has an amino acid sequence represented by SEQ ID NO: 201, while a polynucleotide encoding the same has a nucleotide sequence represented by SEQ ID NO: 200; and HM₁R3M₁ has an amino acid sequence represented by SEQ ID NO: 203, while a polynucleotide encoding the same has a nucleotide sequence represented by SEQ ID NO: 202.

Further, the truncated forms of the cell permeable RUNX3 recombinant protein are as follows:
1) HR3NM₁, where a kFGF4-derived MTD is fused to the C-terminus of a RUNX3 N-terminal domain fragment lacking R-terminal and PST-rich domains,
2) HR3RM₁, where a kFGF4-derived MTD is fused to the C-terminus of a RUNX3 R-terminal domain fragment lacking N-terminal and PST-rich domains,
3) HR3PM₁, where a kFGF4-derived MTD is fused to C-terminus of a RUNX3 PST-rich domain fragment lacking N- and R-terminal domains,
4) HR3NRM₁, where a kFGF4-derived MTD is fused to the C-terminus of a RUNX3 N- and R-terminal domain fragment lacking a PST-rich domain,
5) HR3PRM₁, where a kFGF4-derived MTD is fused to the C-terminus of a RUNX3 R-terminal and PST-rich domain fragment lacking an N-terminal domain, and
6) HR3CRM₁, where a kFGF4-derived MTD is fused to the C-terminus of a portion of a RUNX3 R-terminal and PST-rich domain fragment lacking an N-terminal domain which corresponds to amino acid residues 68-200 in the amino acid sequence of SEQ ID NO: 2
where a His-tag and a NLS derived from SV40 large T antigen are covalently coupled to the N-terminus of the above constructs.

As for the truncated forms of the cell permeable RUNX3 recombinant protein as described above, HR3NM₁ has an amino acid sequence represented by SEQ ID NO: 205, while a polynucleotide encoding the same has a nucleotide sequence represented by SEQ ID NO: 204; HR3RM₁ has an amino acid sequence represented by SEQ ID NO: 207, while a polynucleotide encoding the same has a nucleotide sequence represented by SEQ ID NO: 206; HR3PM₁ has an amino acid sequence represented by SEQ ID NO: 209, while a polynucleotide encoding the same has a nucleotide sequence represented by SEQ ID NO: 208; HR3NRM₁ has an amino acid sequence represented by SEQ ID NO: 211, while a polynucleotide encoding the same has a nucleotide sequence represented by SEQ ID NO: 210; HR3PRM₁ has an amino acid sequence represented by SEQ ID NO: 213, while a polynucleotide encoding the same has a nucleotide sequence represented by SEQ ID NO: 212; and HR3CRM₁ has an amino acid sequence represented by SEQ ID NO: 215, while a polynucleotide encoding the same has a nucleotide sequence represented by SEQ ID NO: 214.

In another embodiment, the present invention relates to the construction of three full-length forms of a cell permeable RUNX3 recombinant protein by using a JO-57 MTD, a JO-85 MTD, a JO-13 MTD and a JO-108 MTD, respectively.

Referring to FIG. 1b, the full-length forms of the cell permeable RUNX3 recombinant protein constructed by using a JO-57 MTD are as follows:
1) HM₂R3, where a JO-57 MTD is fused to the N-terminus of a full-length RUNX3,
2) HR3M₂, where a JO-57 MTD is fused to the C-terminus of a full-length RUNX3, and
3) HM₂R3M₂, where a JO-57 MTD is fused to both termini of a full-length RUNX3,
where a His-tag and a NLS derived from SV40 large T antigen are covalently coupled to the N-terminus of the above constructs.

Further, the full-length forms of the cell permeable RUNX3 recombinant protein constructed by using a JO-85 MTD are as follows:
1) HM₃R3, where a JO-85 MTD is fused to the N-terminus of a full-length RUNX3,
2) HR3M₃, where a JO-85 MTD is fused to the C-terminus of a full-length RUNX3, and
3) HM₃R3M₃, where a JO-85 MTD is fused to both termini of a full-length RUNX3,
where a His-tag and a NLS derived from SV40 large T antigen are covalently coupled to the N-terminus of the above constructs.

Further, the full-length forms of the cell permeable RUNX3 recombinant protein constructed by using a JO-13 MTD are as follows:
1) HM₄R3, where a JO-13 MTD is fused to the N-terminus of a full-length RUNX3,
2) HR3M₄, where a JO-13 MTD is fused to the C-terminus of a full-length RUNX3, and
3) HM₄R3M₄, where a JO-13 MTD is fused to both termini of a full-length RUNX3,
where a His-tag and a NLS derived from SV40 large T antigen are covalently coupled to the N-terminus of the above constructs.

Further, the full-length forms of the cell permeable RUNX3 recombinant protein constructed by using a JO-108 MTD are as follows:
1) HM₅R3, where a JO-108 MTD is fused to the N-terminus of a full-length RUNX3,
2) HR3M₅, where a JO-108 MTD is fused to the C-terminus of a full-length RUNX3, and
3) HM₅R3M₅, where a JO-108 MTD is fused to both termini of a full-length RUNX3,
where a His-tag and a NLS derived from SV40 large T antigen are covalently coupled to the N-terminus of the above constructs.

As for the full-length forms of the cell permeable RUNX3 recombinant protein constructed by using a JO-57 MTD as described above, HM₂R3 has an amino acid sequence represented by SEQ ID NO: 217, while a polynucleotide encoding the same has a nucleotide sequence represented by SEQ ID NO: 216; HR3M₂ has an amino acid sequence represented by SEQ ID NO: 219, while a polynucleotide encoding the same has a nucleotide sequence represented by SEQ ID NO: 218; and HM₂R3M₂ has an amino acid sequence represented by SEQ ID NO: 221, while a polynucleotide encoding the same has a nucleotide sequence represented by SEQ ID NO: 220.

As for the full-length forms of the cell permeable RUNX3 recombinant protein constructed by using a JO-85 MTD as described above, HM₃R3 has an amino acid sequence represented by SEQ ID NO: 223, while a polynucleotide encoding the same has a nucleotide sequence represented by SEQ ID NO: 222; HR3M₃ has an amino acid sequence represented by SEQ ID NO: 225, while a polynucleotide encoding the same has a nucleotide sequence represented by SEQ ID NO: 224; and HM₃R3M₃ has an amino acid sequence represented by SEQ ID NO: 227, while a polynucleotide encoding the same has a nucleotide sequence represented by SEQ ID NO: 226.

As for the full-length forms of the cell permeable RUNX3 recombinant protein constructed by using a JO-13 MTD as described above, HM₄R3 has an amino acid sequence represented by SEQ ID NO: 229, while a polynucleotide encoding the same has a nucleotide sequence represented by SEQ ID NO: 228; HR3M₄ has an amino acid sequence represented by SEQ ID NO: 231, while a polynucleotide encoding the same has a nucleotide sequence represented by SEQ ID NO: 230; and HM₄R3M₄ has an amino acid sequence represented by SEQ ID NO: 233, while a polynucleotide encoding the same has a nucleotide sequence represented by SEQ ID NO: 232.

As for the full-length forms of the cell permeable RUNX3 recombinant protein constructed by using a JO-108 MTD as described above, HM₅R3 has an amino acid sequence represented by SEQ ID NO: 235, while a polynucleotide encoding the same has a nucleotide sequence represented by SEQ ID NO: 234; HR3M₅ has an amino acid sequence represented by SEQ ID NO: 237, while a polynucleotide encoding the same has a nucleotide sequence represented by SEQ ID NO: 236; and HM₅R3M₅ has an amino acid sequence represented by SEQ ID NO: 239, while a polynucleotide encoding the same has a nucleotide sequence represented by SEQ ID NO: 238.

As a control for the cell permeable RUNX3 recombinant proteins, HR3, where a full-length RUNX3 is fused only to a NLS derived from SV40 large T antigen and a histidine-tag (His-Tag) without any MTD, is constructed. The control protein has an amino acid sequence represented by SEQ ID NO: 241, while a polynucleotide encoding the same has a nucleotide sequence represented by SEQ ID NO: 240.

Further, the present invention provides an expression vector containing the polynucleotide encoding each of the cell permeable RUNX3 recombinant proteins described above, and a transformant capable of producing each of the cell permeable RUNX3 recombinant proteins at high levels, which is obtainable by transforming a host cell using the expression vector.

As used herein, the term “expression vector” is a vector capable of expressing a target protein or a target RNA in a suitable host cell. The nucleotide sequence of the present invention may be present in a vector in which the nucleotide sequence is operably linked to regulatory sequences capable of providing for the expression of the nucleotide sequence by a suitable host cell.

Within an expression vector, the term “operably linked” is intended to mean that the nucleotide sequence of interest is linked to the regulatory sequence(s) in a manner which allows for expression of the nucleotide sequence. The term “regulatory sequence” is intended to include promoters, enhancers, and other expression control elements. Such operable linkage with the expression vector can be achieved by conventional gene recombination techniques known in the art, while site-directed DNA cleavage and linkage are carried out by using conventional enzymes known in the art.

The expression vectors suitable for the present invention may include plasmid vectors, cosmid vectors, bacteriophage vectors, viral vectors and the like, but are not limited thereto. The expression vectors for use in the present invention may contain a signal sequence or a leader sequence for membrane targeting or secretion, as well as regulatory sequences such as a promoter, an operator, an initiation codon, a termination codon, a polyadenylation signal, an enhancer and the like. The promoter may be a constitutive or an inducible promoter. Further, the expression vector may include one or more selectable marker genes for selecting the host cell containing the expression vector, and may further include a nucleotide sequence that enables the vector to replicate in the host cell in question.

The expression vector constructed according to the present invention may be exemplified by pHR3M₁ where the polynucleotide encoding the recombinant protein HR3M₁ where a kFGF4-derived MTD is fused to the C-terminus of a full-length RUNX3 is inserted into a cleavage site of NdeI restriction enzyme within the multiple cloning sites (MCS) of a pET-28a(+) vector.

In another embodiment, the polynucleotide of the present invention is cloned into a pET-28a(+) vector (Novagen, Germany) bearing a His-tag sequence so as to fuse six histidine residues to the N-terminus of the cell permeable RUNX3 recombinant protein to allow easy purification.

Accordingly, the cell permeable RUNX3 recombinant protein expressed in the above expression vector has a structure where one of a kFGF4-derived MTD, a JO-57 MTD, a JO-85 MTD, a JO-13 MTD and a JO-108 MTD is fused to the full-length or truncated RUNX3, and a His-tag and NLS are linked to the N-terminus thereof.

The present invention further provides a transformant capable of producing each of the cell permeable RUNX3 recombinant proteins at high levels which is obtainable by transforming a host cell using the expression vector. The host cell suitable for the present invention may be eukaryotic cells, such as E. coli. In one embodiment of the present invention, E. coli used as a host cell is transformed with the expression vector, for example, pHR3M₁ containing the polynucleotide encoding the cell permeable recombinant protein HR3M₁ where a kFGF4-derived MTD is fused to the C-terminus of a full-length RUNX3 according to the present invention so as to produce the cell permeable RUNX3 recombinant protein at high levels. Methods for transforming bacterial cells are well known in the art, and include, but are not limited to, biochemical means such as transformation, transfection, conjugation, protoplast fusion, calcium phosphate-precipitation, and application of polycations such as diethylaminoethyl (DEAE) dextran, and mechanical means such as electroporation, direct microinjection, microprojectile bombardment, calcium phosphate (CaPO₄) precipitation, calcium chloride (CaCl₂) precipitation, PEG-mediated fusion and liposome-mediated method.

In some embodiments, the transformants DH5α/HM₂R3 and DH5α/HM₃R3 obtained by transforming E. coli DH5α with the expression vector containing the cell permeable RUNX3 recombinant protein HM₂R3 where a JO-57 MTD is fused to the N-terminus of a full-length RUNX3, and the expression vector containing the cell permeable RUNX3 recombinant protein HM₃R3 where a JO-85 MTD is fused to the C-terminus thereof, respectively, were deposited under accession numbers KCTC-11408BP and KCTC-11409BP, respectively, with the Korean Collection for Type Cultures (KCTC), Korea Research Institute of Bioscience and Biotechnology (KRIBB), 52, Oun-Dong, Yusong-Ku, Taejon 305-333, Republic of Korea. All deposits referred to herein were made on Oct. 29, 2008 in accordance with the Budapest Treaty, and all restrictions imposed by the depositor on the availability to the public of the deposited biological material will be irrevocably removed upon the granting of the patent.

The present invention provides a method of producing the cell permeable RUNX3 recombinant proteins at high levels, which includes the step of culturing the above transformant.

The method of the present invention may be carried out by culturing the transformant in a suitable medium under suitable conditions for expressing a cell permeable RUNX3 recombinant protein of the present invention in the expression vector introduced into the transformant. Methods for expressing a recombinant protein by culturing a transformant are well known in the art, and for example, may be carried out by inoculating a transformant in a suitable medium for growing the transformant, performing a subculture, transferring the same to a main culture medium, culturing under suitable conditions, for example, supplemented with a gene expression inducer, isopropyl-β-D-thiogalactoside (IPTG) and, thereby, inducing the expression of a recombinant protein. After the culture is completed, it is possible to recover a “substantially pure” recombinant protein from the culture solution. The term “substantially pure” means that the recombinant protein and polynucleotide encoding the same of the present invention are essentially free of other substances with which they may be found in nature or in vivo systems to the extent practical and appropriate for their intended use.

A recombinant protein of the present invention obtained as above may be isolated from the inside or outside (e.g., medium) of host cells, and purified as a substantially pure homogeneous polypeptide. The method for polypeptide isolation and purification is not limited to any specific method. In fact, any standard method may be used. For instance, chromatography, filters, ultrafiltration, salting out, solvent precipitation, solvent extraction, distillation, immunoprecipitation, SDS-polyacrylamide gel electrophoresis, isoelectric point electrophoresis, dialysis, and recrystallization may be appropriately selected and combined to isolate and purify the polypeptide. As for chromatography, affinity chromatography, ion-exchange chromatography, hydrophobic chromatography, gel filtration chromatography, reverse phase chromatography, adsorption chromatography, etc., for example, may be used (Maniatis et al., Molecular Cloning: A Laboratory Manual, Cold Spring Harbor Laboratory, Cold Spring Harbor, N.Y., 1982; Sambrook et al., Molecular Cloning: A Laboratory Manual, 2d Ed., Cold Spring Harbor Laboratory Press, 1989; Deutscher, M., Guide to Protein Purification Methods Enzymology vol. 182. Academic Press. Inc., San Diego, Calif., 1990).

Meanwhile, the recombinant protein expressed in the transformants according to the present invention can be classified into a soluble fraction and an insoluble fraction according to protein characteristics during the protein purification process. If the majority of the expressed recombinant proteins are present in the soluble fraction, the recombinant protein can be isolated and purified according to the method as described above. However, when the majority of the expressed recombinant proteins are present in the insoluble fraction, i.e., as inclusion bodies, the recombinant proteins are first solubilized by using polypeptide denaturing agents, e.g., urea, guanidine HCl, or detergents, and then, purified by performing a series of centrifugation, dialysis, electrophoresis and column chromatography. Since there is the risk of losing the recombinant protein's activity due to a structural modification caused by the polypeptide denaturing agent, the process of purifying the recombinant protein from the insoluble fraction requires desalting and refolding steps. That is, the desalting and refolding steps can be performed by dialysis and dilution with a solution that does not include a polypeptide denaturing agent or by centrifugation with a filter. Further, if a salt concentration of the solution used for the purification of a recombinant protein from a soluble fraction is relatively high, such desalting and refolding steps may be performed.

In some embodiments, it has been found that the cell permeable RUNX3 recombinant protein of the present invention mostly exists in the insoluble fraction as an inclusion body. In order to purify the recombinant protein from the insoluble fraction, the insoluble fraction may be dissolved in a lysis buffer containing a non-ionic surfactant such as Triton X-100, subjected to ultrasonification, and then centrifuged to separate a precipitate. The separated precipitate may be dissolved in a buffer supplemented with a strong denaturing agent, such as urea, and centrifuged to separate the supernatant. The above separated supernatant is purified by means of a histidin-tagged protein purification kit and subjected to ultrafiltration, for example, by using an amicon filter for salt removal and protein refolding, thereby obtaining a purified recombinant protein of the present invention.

Further, the present invention provides an anticancer pharmaceutical composition comprising the cell permeable RUNX3 recombinant protein as an effective ingredient for treating RUNX3 deficiency or failure.

The cell permeable RUNX3 recombinant proteins of the present invention can reactivate a TGF-β signal transduction pathway by efficiently introducing a tumor and metastasis suppressor protein RUNX3 into a cell when the protein is deficient or its function is lost. Therefore, the cell permeable RUNX3 recombinant proteins of the present invention can be effectively used as an anticancer agent capable of preventing and/or treating cancer growth and metastasis.

The pharmaceutical composition comprising the recombinant protein of the present invention as an effective ingredient may further include pharmaceutically acceptable carriers suitable for oral administration or parenteral administration. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's Pharmaceutical Sciences, 19th ed., Mack Publishing Company, Easton, Pa., 1995). The carriers for oral administration may include lactose, starch, cellulose derivatives, magnesium stearate, stearic acid and the like. In case of oral administration, the recombinant protein of the present invention can be formulated in the form of chewable tablets, buccal tablets, troches, capsules, elixir, suspensions, syrup, wafers or combination thereof by mixing with the carriers. Further, the carriers for parenteral administration may include water, suitable oil, saline, aqueous glucose, glycol and the like, and may further include stabilizers and preservatives. The stabilizers suitable for the present invention may include antioxidants such as sodium bisulfite, sodium sulfite and ascorbic acid. Suitable preservatives may include benzalconium chloride, methly-paraben, propyl-paraben and chlorobutanol.

The pharmaceutical composition of the present invention may be formulated into various parenteral or oral administration forms. Representative examples of the parenteral formulation include those designed for administration by injection. For injection, the recombinant proteins of the present invention may be formulated in aqueous solutions, specifically in physiologically compatible buffers or physiological saline buffer. These injection formulations may be formulated by conventional methods using one or more dispersing agents, wetting agents and suspending agents. For oral administration, the proteins can be readily formulated by combining the proteins with pharmaceutically acceptable carriers well known in the art. Such carriers enable the proteins of the invention to be formulated as tablets, pills, capsules, liquids, gels, syrups, slurries, suspensions and the like, for oral ingestion by a patient to be treated. Such oral solid formulations may include suitable excipients such as diluents (e.g., lactose, dextrose, sucrose, mannitol, sorbitol cellulose and/or glycin) and lubricants (e.g., colloidal silica, talc, stearic acid, magnesium stearate, calcium stearate, and/or polyethylene glycol). The tablets may include binders, such as aluminum silicate, starch, gelatin, gum tragacanth, methyl cellulose, hydroxypropylmethyl-cellulose, sodium carboxymethylcellulose, and/or polyvinylpyrrolidone (PVP), and disintegrating agents, such as cross-linked polyvinylpyrrolidone, agar, or alginic acid or a salt thereof such as sodium alginate. If desired, absorbents, coloring agents, flavoring agents and/or sweeteners may be added. The formulations can be prepared by mixing, granulating or coating according to conventional methods well-known in the art.

If necessary, the pharmaceutical compositions of the present invention may further include pharmaceutical additives, such as preservatives, antioxidants, emulsifiers, buffering agents and/or salts for regulating osmosis and other therapeutically effective materials, and can be formulated according to conventional methods known in the art.

In addition, the pharmaceutical composition of the present invention can be administered via oral routes or parenteral routes such as intravenously, subcutaneously, intranasally or intraperitoneally. The oral administration may include sublingual application. The parenteral administration may include drip infusion and injection such as subcutaneous injection, intramuscular injection, intravenous injection and introtumoral injection.

The total effective amount of the recombinant protein of the present invention can be administered to patients in a single dose or can be administered by a fractionated treatment protocol, in which multiple doses are administered over a more prolonged period of time. Although the amount of the recombinant protein or nucleic acid encoding the same in the pharmaceutical composition of the present invention may vary depending on the severity of diseases, the protein or the nucleic acid may be generally administered several times a day at an effective dose of 5 to 20 mg. However, a suitable dose of the recombinant protein in the pharmaceutical composition of the present invention may depend on many factors, such as age, body weight, health condition, sex, disease severity, diet and excretion of patients, as well as the route of administration and the number of treatments to be administered. In view of the above factors, any person skilled in the art may determine the effective dose of the recombinant protein as an anti-metastatic agent for preventing metastasis in various human cancers. The pharmaceutical composition of the present invention containing the recombinant protein has no special limitations on its formulation, administration route and/or administration mode insofar as it exhibits the effects of the present invention.

### EXAMPLES

The following examples are provided to illustrate the embodiments of the present invention in more detail, but are by no means intended to limit its scope.

### Example 1

### Construction of Cell Permeable RUNX3 Recombinant Proteins (CP-RUNX3)<

1-1>Construction of Cell Permeable RUNX3 Recombinant Proteins Using a kFGF4-Derived MTD

Three full-length forms and six truncated forms of a cell permeable RUNX3 recombinant protein were constructed by using a kFGF4-derived MTD (MTD₁).

Referring to FIG. 1, the full-length forms of CP-RUNX3 recombinant constructs were as follows:
1) HM₁R3, where a kFGF4-derived MTD is fused to the N-terminus of a full-length RUNX3,
2) HR3M₁, where a kFGF4-derived MTD is fused to the C-terminus of a full-length RUNX3, and
3) HM₁R3M₁ where a kFGF4-derived MTD is fused to both termini of a full-length RUNX3,
where a His-tag and a NLS derived from SV40 large T antigen are covalently coupled to the N-terminus of the above constructs.

In order to prepare the full-length CP-RUNX3 recombinant constructs, polymerase chain reactions (PCRs) were carried out by using the oligonucleotides as a primer pair specific for each recombinant construct and a human RUNX3 cDNA as a template. The forward and reverse primers for amplifying HM₁R3 have nucleotide sequences represented by SEQ ID NOS: 244 and 243, respectively; those for amplifying HR3M₁ have nucleotide sequences represented by SEQ ID NOS: 242 and 245, respectively; and those for amplifying HM₁R3M₁ have nucleotide sequences represented by SEQ ID NOS: 244 and 245, respectively.

Further, the truncated forms of a CP-RUNX3 recombinant protein were as follows:
1) HR3NM₁, where a kFGF4-derived MTD is fused to the C-terminus of a RUNX3 N-terminal domain fragment lacking R-terminal and PST-rich domains,
2) HR3RM₁, where a kFGF4-derived MTD is fused to the C-terminus of a RUNX3 R-terminal domain fragment lacking N-terminal and PST-rich domains,
3) HR3PM₁, where a kFGF4-derived MTD is fused to the C-terminus of a RUNX3 PST-rich domain fragment lacking N- and R-terminal domains,
4) HR3NRM₁, where a kFGF4-derived MTD is fused to the C-terminus of a RUNX3 N- and R-terminal domain fragment lacking a PST-rich domain,
5) HR3PRM₁, where a kFGF4-derived MTD is fused to the C-terminus of a RUNX3 R-terminal and PST-rich domain fragment lacking an N-terminal domain, and
6) HR3CRM₁, where a kFGF4-derived MTD is fused to the C-terminus of a portion of a RUNX3 R-terminal and PST-rich domain fragment lacking an N-terminal domain which corresponds to amino acid residues 68-200 in the amino acid sequence of SEQ ID NO: 2
where a His-tag and a NLS derived from SV40 large T antigen are covalently coupled to the N-terminus of the above constructs.

In order to prepare the truncated CP-RUNX3 recombinant proteins, PCR was carried out by using the oligonucleotides as a primer set specific for each recombinant protein and a human RUNX3 cDNA as a template. The forward and reverse primers for amplifying HR3NM₁ have nucleotide sequences represented by SEQ ID NOS: 246 and 247, respectively; while those for amplifying HR3RM₁ have nucleotide sequences represented by SEQ ID NOS: 248 and 249, respectively; those for amplifying HR3PM₁ have nucleotide sequences represented by SEQ ID NOS: 250 and 245, respectively; those for amplifying HR3NRM₁ have nucleotide sequences represented by SEQ ID NOS: 246 and 249, respectively; those for amplifying HR3RPM₁ have nucleotide sequences represented by SEQ ID NOS: 248 and 245, respectively; and those for amplifying HR3CRM₁ have nucleotide sequences represented by SEQ ID NOS: 251 and 252, respectively

The PCR was performed in a 50 μl reaction mixture containing 100 ng of human RUNX3 cDNA (College of Medicine, Chungbuk National University) as a template, 0.2 mM dNTP mixture, 1 μM of each primer, 5 μl of 10× Taq buffer, 1 μl of Taq polymerase (Novagen, Germany). The PCR was performed for 25 cycles at 94° C. for 20 seconds, at 63° C. for 30 seconds and at 72° C. for 30 seconds after the initial denaturation of 94° C. for 5 minutes, followed by the final extension at 72° C. for 5 minutes. After the PCR was completed, the amplified PCR product was digested with restriction enzyme NdeI and loaded onto a 1.0% agarose gel and fractionated.

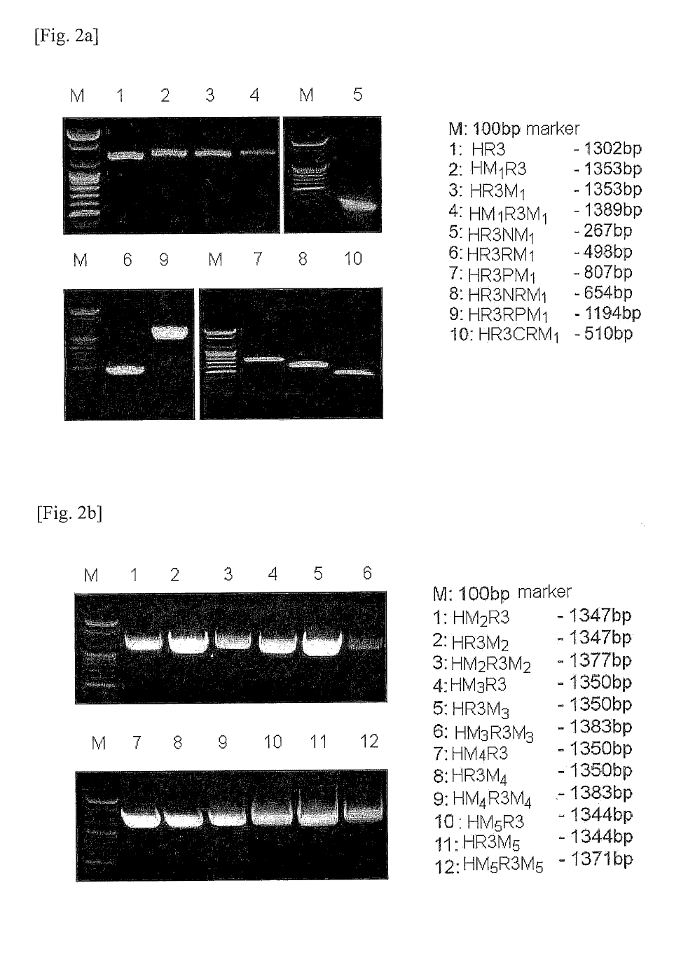

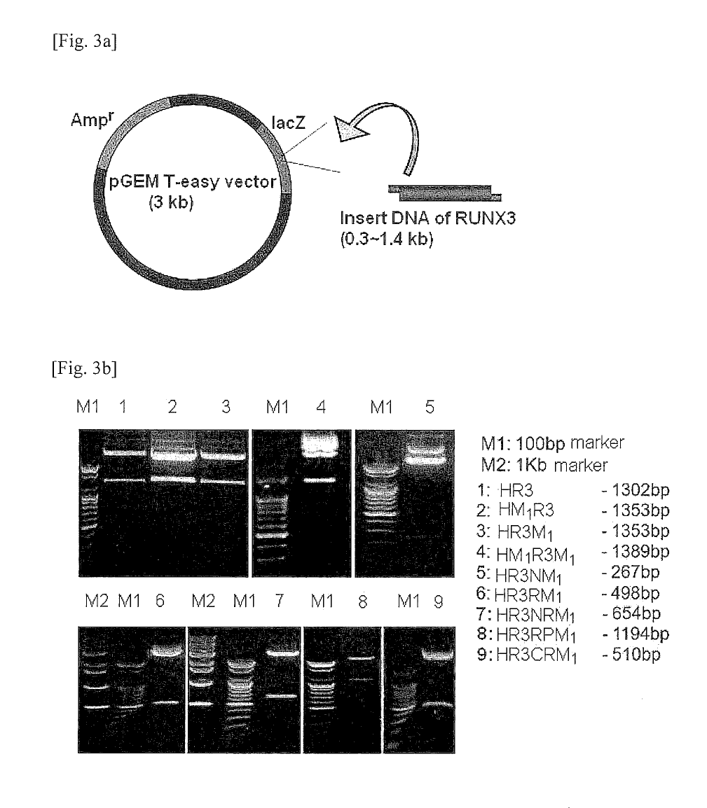

As shown in FIG. 2a, it was confirmed that the expected fragment for each recombinant construct fused to a kFGF4-derived MTD was successfully amplified.

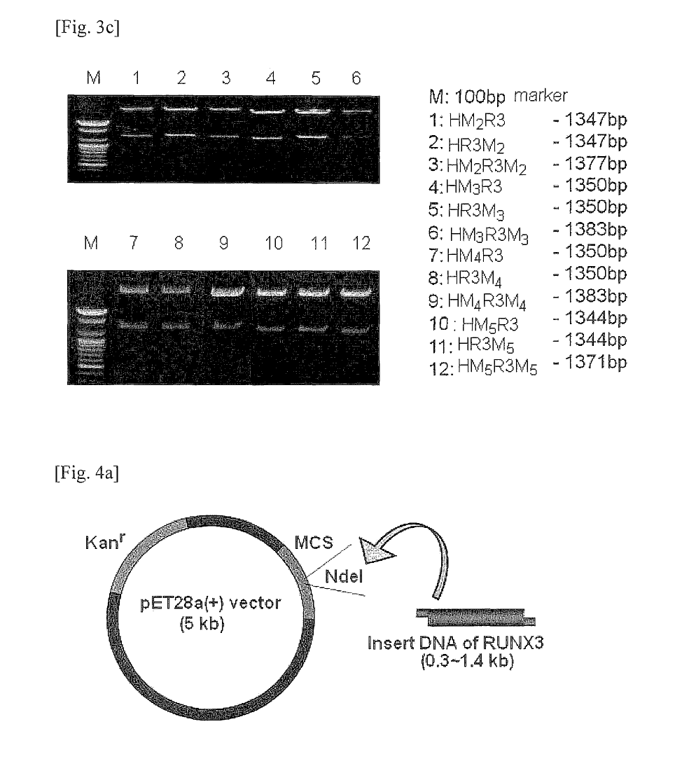

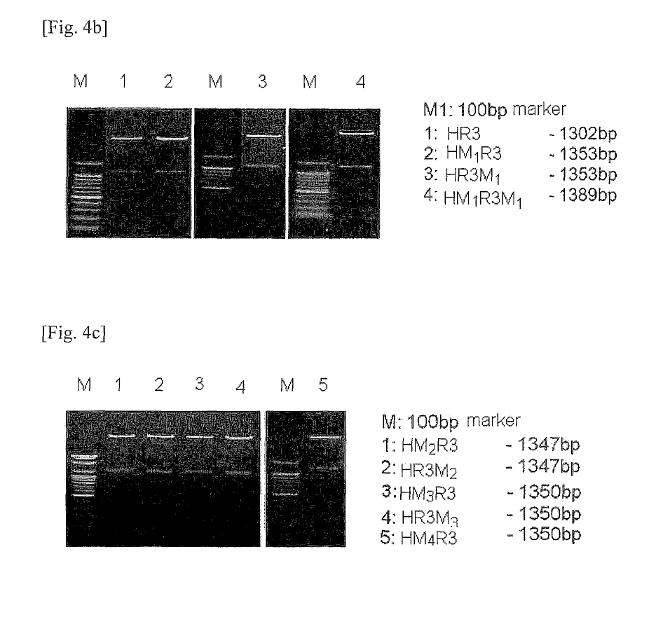

The DNA band of expected size was excised from the gel, eluted, and purified by using a QIAquick Gel extraction kit (Qiagen, USA). The eluted DNA was precipitated with ethanol and resuspended in distilled water for ligation. As shown in FIG. 3a, the PCR amplified DNA fragment containing the coding region was subcloned into a pGEM-T Easy vector (Promega, USA) with a T4 ligase according to the TA cloning method, and then, followed by transformation of E. coli DH5α competent cells with the pGEM-T Easy vector. The cells were plated onto LB plate media supplemented with 100 μg/ml of ampicillin and cultured at 37° C. for overnight. After the recombinant fragment-inserted pGEM-T Easy vector was isolated by treating with restriction enzyme NdeI 37° C. for 1 hour, it was subjected to a 0.8% agarose gel electrophoresis.

As shown in FIG. 3b, it was comfirmed that the insert DNA of the CP-RUNX3 recombinant construct was appropriately subcloned into the pGEM-T Easy vector.

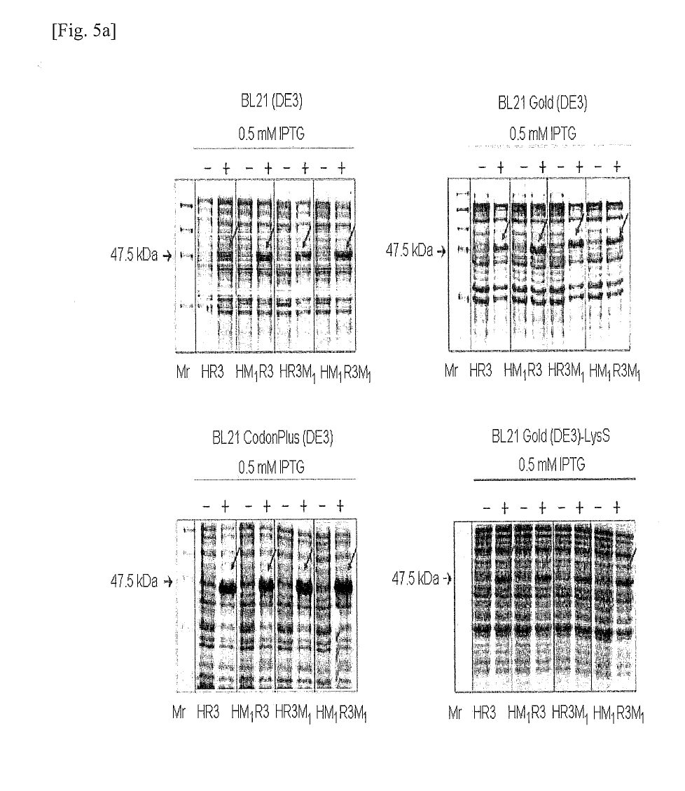

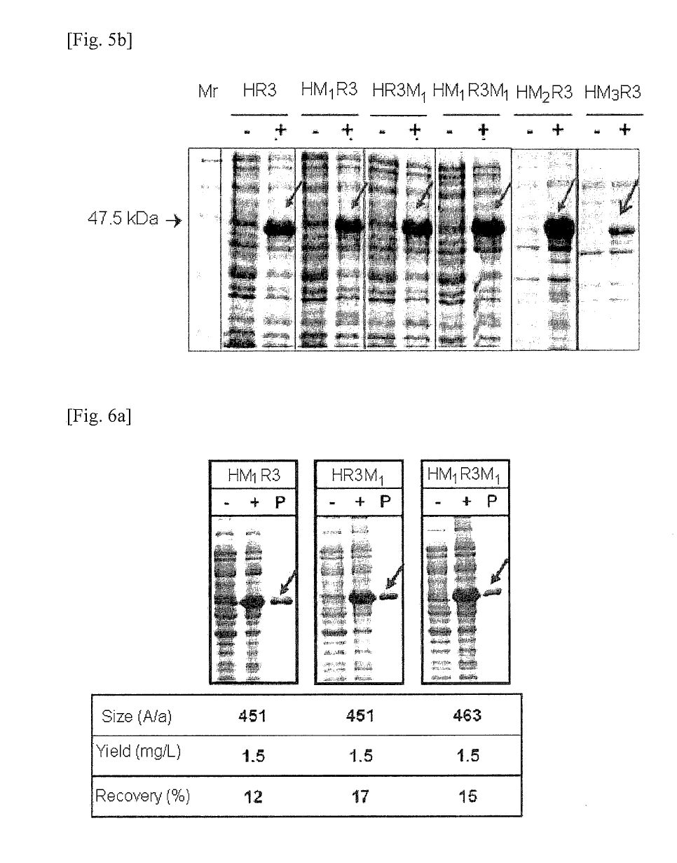

A pET-28(+)a vector (Novagen, Germany) bearing a histidine-tag and a T7 promoter was digested with a restriction enzyme NdeI for 1 hour at 37° C. The pGEM-T Easy vector fragments containing the CP-RUNX3 recombinant fragment and pET-28(+)a vector fragment were purified by using a QIAquick Gel extraction kit. Each of the pGEM-T Easy vector fragments was cloned into the pre-treated pET-28a(+) with a T4 ligase at 16 r for 12 hours, followed by transformation of E. coli DH5α competent cells with the resulting pET-28a(+) vector (FIG. 4a).

After the clones were treated with the restriction enzyme NdeI (Enzynomics, Korea) and subjected to 0.8% agarose gel electrophoresis, it was verified that the cloning of the insert DNA of CP-RUNX3 recombinant construct into pET-28a(+) vector, as shown in FIG. 4b.

The successfully cloned expression vectors for expressing cell permeable RUNX3 recombinant proteins were designated pHM₁R3, pHR3M₁, pHM₁R3M₁, pHR3NM₁, pHR3RM₁, pHR3PM₁, pHR3NRM₁, pHR3RPM₁, and pHR3CRM₁, respectively.

The results of sequencing analysis are as follows:

As for the full-length forms of the cell permeable RUNX3 recombinant protein as described above, HM₁R3 has an amino acid sequence represented by SEQ ID NO: 199, while a polynucleotide encoding the same has a nucleotide sequence represented by SEQ ID NO: 198; HR3M₁ has an amino acid sequence represented by SEQ ID NO: 201, while a polynucleotide encoding the same has a nucleotide sequence represented by SEQ ID NO: 200; and HM₁R3M₁ has an amino acid sequence represented by SEQ ID NO: 203, while a polynucleotide encoding the same has a nucleotide sequence represented by SEQ ID NO: 202.

As for the truncated forms of the cell permeable RUNX3 recombinant protein as described above, HR3NM₁ has an amino acid sequence represented by SEQ ID NO: 205, while a polynucleotide encoding the same has a nucleotide sequence represented by SEQ ID NO: 204; HR3RM₁ has an amino acid sequence represented by SEQ ID NO: 207, while a polynucleotide encoding the same has a nucleotide sequence represented by SEQ ID NO: 206; HR3PM₁ has an amino acid sequence represented by SEQ ID NO: 209, while a polynucleotide encoding the same has a nucleotide sequence represented by SEQ ID NO: 208; 1-1R3NRM₁ has an amino acid sequence represented by SEQ ID NO: 211, while a polynucleotide encoding the same has a nucleotide sequence represented by SEQ ID NO: 210; HR3PRM₁ has an amino acid sequence represented by SEQ ID NO: 213, while a polynucleotide encoding the same has a nucleotide sequence represented by SEQ ID NO: 212; and HR3CRM₁ has an amino acid sequence represented by SEQ ID NO: 215, while a polynucleotide encoding the same has a nucleotide sequence represented by SEQ ID NO: 214.

As a control for the cell permeable RUNX3 recombinant proteins, HR3, where a full-length RUNX3 is fused only to a nuclear localization sequence (NLS) derived from SV40 large T antigen and a histidine-tag (His-Tag) without any MTD, was constructed. The control protein has an amino acid sequence represented by SEQ ID NO: 241, while a polynucleotide encoding the same has a nucleotide sequence represented by SEQ ID NO: 240.

### <1-2>Construction of Cell Permeable RUNX3 Recombinant Proteins Using one of JO-57, JO-85, JO-13 and JO-108 MTDs

In order to construct a cell permeable RUNX3 recombinant protein by using one of a JO-57 MTD (MTD₂), a JO-85 MTD (MTD₃), a JO-13 MTD (MTD₄) and a JO-108 MTD (MTD₅), three full-length forms of a CP-RUNX3 recombinant construct for each MTD were constructed.

Referring to FIG. 2b, the full-length forms of the CP-RUNX3 recombinant constructs being fused to a JO-57 MTD were as follows:
1) HM₂R3, where a JO-57 MTD is fused to the N-terminus of a full-length RUNX3;
2) HR3M₂, where a JO-57 MTD is fused to the C-terminus of a full-length RUNX3; and
3) HM₂R3M₂, where a JO-57 MTD is fused to both termini of a full-length RUNX3,
where a His-tag and a NLS derived from SV40 large T antigen are covalently coupled to the N-terminus of the above constructs.

In order to prepare said full-length CP-RUNX3 recombinant proteins, PCR was carried out according to the same method as described in section <1-1> of Example 1 above. The forward and reverse primers for amplifying HM₂R3 have nucleotide sequences represented by SEQ ID NOS: 253 and 243, respectively; those for amplifying HR3M₂ have nucleotide sequences represented by SEQ ID NOS: 242 and 254, respectively; and those for amplifying HM₂R3M₂ have nucleotide sequences represented by SEQ ID NOS: 253 and 254, respectively.

Further, the full-length forms of a CP-RUNX3 recombinant construct being fused to a JO-85 MTD were as follows:
1) HM₃R3, where a JO-85 MTD is fused to the N-terminus of a full-length RUNX3;
2) HR3M₃, where a JO-85 MTD is fused to the C-terminus of a full-length RUNX3; and
3) HM₃R3M₃, where a JO-85 MTD is fused to both termini of a full-length RUNX3,
where a His-tag and a NLS derived from SV40 large T antigen are covalently coupled to the N-terminus of the above constructs.

In order to prepare said full-length CP-RUNX3 recombinant proteins, PCR was carried out according to the same method as described in section <1-1> of Example 1 above. The forward and reverse primers for amplifying HM₃R3 have nucleotide sequences represented by SEQ ID NOS: 255 and 243, respectively; those for amplifying HR3M₃ have nucleotide sequences represented by SEQ ID NOS: 242 and 256, respectively; and those for amplifying HM₃R3M₃ have nucleotide sequences represented by SEQ ID NOS: 255 and 256, respectively.

Further, the full-length forms of a CP-RUNX3 recombinant construct being fused to a JO-13 MTD were as follows:

1) HM₄R3, where a JO-13 MTD is fused to the N-terminus of a full-length RUNX3;
2) HR3M₄, where a JO-13 MTD is fused to the C-terminus of a full-length RUNX3; and
3) HM₄R3M₄, where a JO-13 MTD is fused to both termini of a full-length RUNX3,
where a His-tag and a NLS derived from SV40 large T antigen are covalently coupled to the N-terminus of the above constructs.

In order to prepare said full-length CP-RUNX3 recombinant proteins, PCR was carried out according to the same method as described in section <1-1> of Example 1 above. The forward and reverse primers for amplifying HM₄R3 have nucleotide sequences represented by SEQ ID NOS: 257 and 243, respectively; those for amplifying HR3M₄ have nucleotide sequences represented by SEQ ID NOS: 242 and 258, respectively; and those for amplifying HM₄R3M₄ have nucleotide sequences represented by SEQ ID NOS: 257 and 258, respectively.

Further, the full-length forms of a CP-RUNX3 recombinant construct being fused to a JO-108 MTD were as follows:
1) HM₅R3, where a JO-108 MTD is fused to the N-terminus of a full-length RUNX3;
2) HR3M₅, where a JO-108 MTD is fused to the C-terminus of a full-length RUNX3; and
3) HM₅R3M₅, where a JO-108 MTD is fused to both termini of a full-length RUNX3,
where a His-tag and a NLS derived from SV40 large T antigen are covalently coupled to the N-terminus of the above constructs.

In order to prepare said full-length CP-RUNX3 recombinant proteins, PCR was carried out according to the same method as described in section <1-1> of Example 1 above. The forward and reverse primers for amplifying HM₅R3 have nucleotide sequences represented by SEQ ID NOS: 259 and 243, respectively; those for amplifying HR3M₅ have nucleotide sequences represented by SEQ ID NOS: 242 and 260, respectively; and those for amplifying HM₅R3M₅ have nucleotide sequences represented by SEQ ID NOS: 259 and 260, respectively.

Each of the PCR amplified DNA fragments was subcloned into a pGEM-T Easy vector, followed by cloning into a pET-28(+)a vector according to the same method as described in section <1-1> of Example 1 above, to thereby obtain expression vectors for expressing cell permeable RUNX3 recombinant proteins. The successful insertion of the recombinant fragment into the pGEM-T Easy and pET-28(+)a vectors is confirmed in FIGS. 3c and 4c.

The thus obtained expression vectors for expressing cell permeable RUNX3 recombinant proteins were designated pHM₂R3, pHR3M₂, pHM₂R3M₂, pHM₃R3, pHR3M₃, pHM₃R3M₃, pHM₄R3, pHR3M₄, pHM₄R3M₄, pHM₅R3, pHR3M₅, and pHM₅R3M₅, respectively.

Among them, the E. coli transformants DH5α/HM₂R3 and DH5α/HM₃R3 obtained by transforming E. coli DH5α with each of the expression vectors pHM₂R3 where a JO-57 MTD is fused to the N-terminus of a full-length RUNX3 and pHM₃R3 where a JO-85 MTD is fused to the C-terminus thereof were deposited on Oct. 29, 2008 in accordance with the Budapest Treaty under accession numbers KCTC-11408BP and KCTC-11409BP, respectively, with the Korean Collection for Type Cultures (KCTC), Korea Research Institute of Bioscience and Biotechnology (KRIBB), 52, Oun-Dong, Yusong-Ku, Taejon 305-333, Republic of Korea.

The results of sequencing analysis are as follows:

As for the full-length forms of the cell permeable RUNX3 recombinant protein constructed by using a JO-57 MTD as described above, HM₂R3 has an amino acid sequence represented by SEQ ID NO: 217, while a polynucleotide encoding the same has a nucleotide sequence represented by SEQ ID NO: 216; HR3M₂ has an amino acid sequence represented by SEQ ID NO: 219, while a polynucleotide encoding the same has a nucleotide sequence represented by SEQ ID NO: 218; and HM₂R3M₂ has an amino acid sequence represented by SEQ ID NO: 221, while a polynucleotide encoding the same has a nucleotide sequence represented by SEQ ID NO: 220.

As for the full-length forms of the cell permeable RUNX3 recombinant protein constructed by using a JO-85 MTD as described above, HM₃R3 has an amino acid sequence represented by SEQ ID NO: 223, while a polynucleotide encoding the same has a nucleotide sequence represented by SEQ ID NO: 222; HR3M₃ has an amino acid sequence represented by SEQ ID NO: 225, while a polynucleotide encoding the same has a nucleotide sequence represented by SEQ ID NO: 224; and HM₃R3M₃ has an amino acid sequence represented by SEQ ID NO: 227, while a polynucleotide encoding the same has a nucleotide sequence represented by SEQ ID NO: 226.

As for the full-length forms of the cell permeable RUNX3 recombinant protein constructed by using a JO-13 MTD as described above, HM₄R3 has an amino acid sequence represented by SEQ ID NO: 229, while a polynucleotide encoding the same has a nucleotide sequence represented by SEQ ID NO: 228; HR3M₄ has an amino acid sequence represented by SEQ ID NO: 231, while a polynucleotide encoding the same has a nucleotide sequence represented by SEQ ID NO: 230; and HM₄R3M₄ has an amino acid sequence represented by SEQ ID NO: 233, while a polynucleotide encoding the same has a nucleotide sequence represented by SEQ ID NO: 232.

As for the full-length forms of the cell permeable RUNX3 recombinant protein constructed by using a JO-108 MTD as described above, HM₅R3 has an amino acid sequence represented by SEQ ID NO: 235, while a polynucleotide encoding the same has a nucleotide sequence represented by SEQ ID NO: 234; HR3M₅ has an amino acid sequence represented by SEQ ID NO: 237, while a polynucleotide encoding the same has a nucleotide sequence represented by SEQ ID NO: 236; and HM₅R3M₅ has an amino acid sequence represented by SEQ ID NO: 239, while a polynucleotide encoding the same has a nucleotide sequence represented by SEQ ID NO: 238.

The oligonucleotides as a forward and reverse primer set specific for each recombinant protein used in Examples <1-1> and <1-2> are summarized in Table 2 below.

TABLE 2

| | SEQ ID | |
|---|---|---|
| Primer | NO | Sequence |
| HR-5′ | 242 | CCGCATATGAAGAAGAAGAGGAAGCGTATTCCCGTAGACC |
| (51 nts) | | CAAGCACCAGC |
| HR-3′ | 243 | CCGCATATGTCAGTAGGGCCGCCACACGGCCTC |
| (33 nts) | | |
| HM₁R5′ | 244 | CCGCATATGAAGAAGAAGAGGAAGGCAGCCGTTCTTCTCC |
| (87 nts) | | CTGTTCTTCTTGCCGCACCCCGTATTCCCGTAGACCCAAGC |
| | | ACCAGC |
| HRM₁-3′ | 245 | CCGCATATGTCAGGGTGCGGCAAGAAGAACAGGGAGAAG |
| (75 nts) | | AACGGCTGCGTAGGGCCGCCACACGGCCTCATCCAT |
| HR-N-5′ | 246 | CCGCATATGAAGAAGAAGAGGAAGCGTATTCCCGTAGACC |
| (51 nts) | | CAAGCACCAGC |
| HR-N-M₁-3′ | 247 | CCGCATATGTCAGGGTGCGGCAAGAAGAACAGGGAGAAG |
| (75 nts) | | AACGGCTGCGCGCACCTCGGGCCGGGCGCGCCCTCC |
| HR-R-5′ | 248 | CCGCATATGAAGAAGAAGAGGAAGTCGATGGTGGACGTGC |
| (51 nts) | | TGGCGGACCAC |
| HR-R-M₁-3′ | 249 | CCGCATATGTCAGGGTGCGGCAAGAAGAACAGGGAGAAG |
| (75 nts) | | AACGGCTGCCCGTCTGGGCTCCCGGGGTCCGTCCAC |
| HR-P-5′ | 250 | CCGCATATGAAGAAGAAGAGGAAGCACCGGCAGAAGCTG |
| (51 nts) | | GAGGACCAGACC |
| HR-CR-5′ | 251 | CCGCATATGAAGAAGAAGAGGAAGCGCACCGACAGCCCCA |
| (51 nts) | | ACTTCCTCTGC |
| HR-CR-M₁-3′ | 252 | CCGCATATGTCAGGGTGCGGCAAGAAGAACAGGGAGAAG |
| (75 nts) | | AACGGCTGCGTCCCCAAAGCGGTCAGGGAACGGCTT |
| HM₂R-5′ | 253 | CCGCATATGAAGAAGAAGAGGAAGCTGATTGCGCTGCTGG |
| (81 nts) | | CGGCGCCGCTGGCGCGTATTCCCGTAGACCCAAGCACCAG |
| | | C |
| HRM₂-3′ | 254 | CCGCATATGTCACGCCAGCGGCGCCGCCAGCAGCGCAATC |
| (69 nts) | | AGGTAGGGCCGCCACACGGCCTCATCCAT |
| HM₃R-5′ | 255 | CCGCATATGAAGAAGAAGAGGAAGCTGCTGGCGGCGGCGG |
| (84 nts) | | CGGCGCTGCTGCTGGCGCGTATTCCCGTAGACCCAAGCACC |
| | | AGC |
| HRM₃-3′ | 256 | CCGCATATGTCACGCCAGCAGCAGCGCCGCCGCCGCCGCC |
| (72 nts) | | AGCAGGTAGGGCCGCCACACGGCCTCATCCAT |
| HM₄R-5′ | 257 | CCGCATATGAAGAAGAAGAGGAAGCTGGCGGCGGCGGCG |
| (84 nts) | | CTGGCGGTGCTGCCGCTGCGTATTCCCGTAGACCCAAGCAC |
| | | CAGC |
| HRM₄-3′ | 258 | CCGCATATGTCACAGCGGCAGCACCGCCAGCGCCGCCGCC |
| (72 nts) | | GCCAGGTAGGGCCGCCACACGGCCTCATCCAT |
| HM₅R-5′ | 259 | CCGCATATGAAGAAGAAGAGGAAGGCGCTGCTGGCGGCGC |
| (78 nts) | | TGCTGGCGCCGCGTATTCCCGTAGACCCAAGCACCAGC |
| HRM₅-3′ | 260 | CCGCATATGTCACGGCGCCAGCAGCGCCGCCAGCAGCGCG |
| (66 nts) | | TAGGGCCGCCACACGGCCTCATCCAT |

### Example 2

### Expression of Recombinant Proteins

### <2-1>Selection of Optimal Bacterial Strains

To select the optimal bacterial strain for the expression of cell permeable RUNX3 recombinant proteins prepared in Example 1 above, the following experiments were carried out in E. coli BL21(DE3), BL21-Gold(DE3), BL21-CodonPlus(DE3) and BL21-Gold(DE3) pLysS strains (Stratagene, USA), all of which contain the LacI promoter.

First, each of the expression vectors pHM₁R3, pHR3M₁, pHM₁R3M₁, and pHR3 (control) was transformed into E. coli BL21(DE3), BL21-Gold(DE3), BL21-CodonPlus(DE3) and BL21-Gold(DE3) pLysS strains, respectively, according to the heat shock method. After the transformation, the cells were cultured in an LB agar plate containing 50 μg/ml of kanamycin. Colonies formed on the plate were grown in 1 ml of LB medium at 37° C. overnight, followed by culturing at 37° C. in 100 ml of LB medium with vigorous shaking until the optical density 600 (OD₆₀₀) reached 0.5. IPTG (isopropyl-β-D-thiogalactoside) was then added thereto at a final concentration of 0.7 mM to induce the expression of the CP-RUNX3 recombinant proteins. Protein induction was prolonged for 3 hours at 37° C. The E. coli culture solutions were harvested by centrifugation at 13,000×g for 1 minute, resuspended in a sample loading buffer (125 mM Tris-HCl, 20% glycerol, 2% (3-mercaptoethanol, 0.04% bromophenol blue, 4% SDS, pH 6.8), and subjected to boiling at 100° C. for 5 minutes. The cell lysates were centrifuged at 13,000 rpm for 1 minute, so as to separate an insoluble fraction from a soluble fraction. The thus obtained soluble and insoluble fractions of CP-RUNX3 recombinant proteins expressed in the E. coli strain with IPTG were loaded on a SDS-PAGE gel.

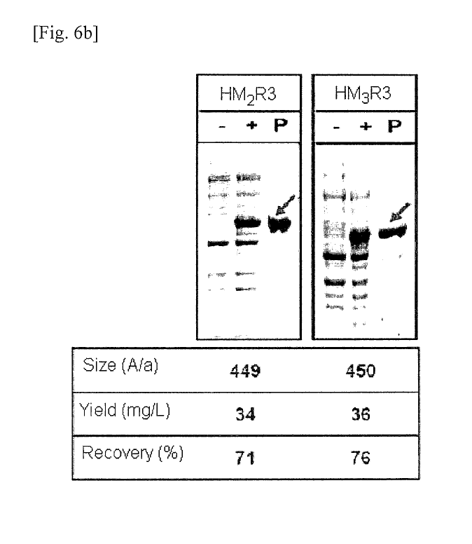

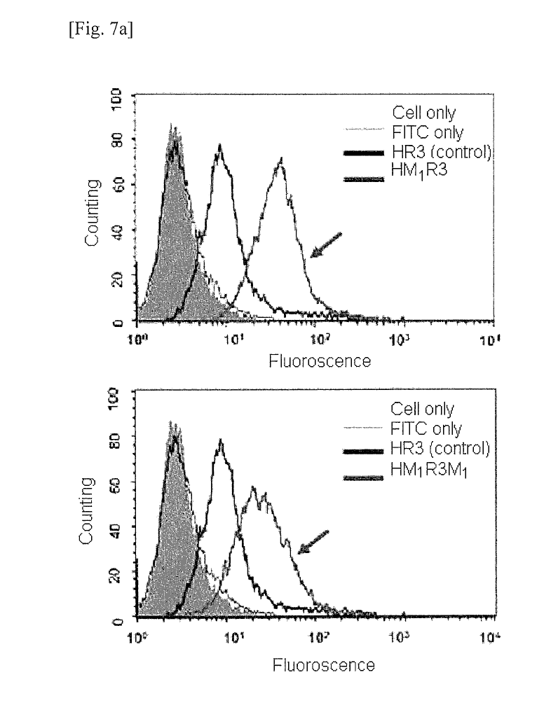

As shown in FIG. 5a, as a result of examining the expression of the recombinant protein according to the present invention in various kinds of host strains, it was found that most cell permeable RUNX3 recombinant proteins showed the highest expression level in BL21 CodonPlus(DE3). According to said result, BL21 CodonPlus(DE3) was selected as the optimal strain for the expression of the cell permeable RUNX3 recombinant proteins according to the present invention.

### <2-2>Inducible Expression of Recombinant Proteins

Each of the expression vectors pHR3 (control), pHM₁R3, pHR3M₁, pHM₁R3M₁, pHM₂R3 and pHM₃R3 was transformed into E. coli BL21 CodonPlus(DE3), selected as the optimal strain in section <2-1> of Example 2 above, according to the heat shock method, followed by culturing in an LB medium containing 50 μg/ml of kanamycin. After that, the cells transformed with the recombinant protein encoding gene were grown in 1 ml of LB medium at 37° C. overnight, followed by culturing at 37° C. in 100 ml of LB medium with vigorous shaking until the optical density 600 (OD₆₀₀) reached 0.5. IPTG was then added thereto at a final concentration of 0.5 mM to induce the expression of the CP-RUNX3 recombinant proteins. Protein induction was prolonged for 3 hours at 37° C. The E. coli culture solutions were harvested by centrifugation at 13,000 rpm for 1 minute, resuspended in a a sample loading buffer (125 mM Tris-HCl, 20% glycerol, 2% β-mercaptoethanol, 0.04% bromophenol blue. 4% SDS, pH 6.8), and subjected to boiling at 100° C. for 5 minutes. The cell lysates were centrifuged at 13,000 rpm for 1 minute, so as to separate the insoluble fraction from the soluble fraction. The thus obtained soluble and insoluble fractions of CP-RUNX3 recombinant proteins expressed in the E. coli strain with IPTG were loaded on a SDS-PAGE gel.

As shown in FIG. 5b, it was confirmed that the cell permeable RUNX3 recombinant proteins (˜47.5 kDa) expressed in the host cell were mostly included in the insoluble fraction as an inclusion body, and their expression was significantly increased in the presence of IPTG (+) as compared with in the absence of IPTG (−).

### Example 3

### Purification of Recombinant Proteins

The inducible expression of cell permeable RUNX3 recombinant proteins in an E. coli system leads to the formation of insoluble aggregates, which are known as inclusion bodies. To completely solubilize these inclusion bodies, all of the above expressed proteins were denatured by dissolving them in SDS used as a strong denaturing agent.

First, the BL21 CodonPlus(DE3) strains transformed with each of the expression vectors pHM₁R3, pHR3M₁, pHM₁R3M₁, pHM₂R3 and pHM₃R3 were cultured in 1 l of an LB medium as described in Example 2. Each culture solution was harvested by centrifugation, gently resuspended in 100 ml of a washing buffer (100 mM Tris-HCl, 5 mM EDTA, pH 8.0) without forming bubbles, and subjected to standing for 15 minutes at room temperature. After to the cell suspension was added 0.1 g of sodium deoxycholate, the mixture was subjected to pippetting so as to uniformly mix and ultrasonication on ice using a sonicator equipped with a microtip. The cells were intermittently sonicated for 30 seconds, followed by cooling for 10 seconds, while setting the power to 27% of the maximum power. The total sonication time was 10 minutes. The cell lysates were centrifuged at 4° C., 8,000×g for 10 minutes, so as to separate the supernatant and the cell precipitate. The cell precipitate was resuspended in 100 ml of a washing buffer (100 mM Tris-HCl, 0.1% sodium dexoycholate, 5 mM EDTA, pH 8.0) without forming bubbles, and was centrifuged at 4° C., 8,000×g for 10 minutes, so as to separate the supernatant and the cell precipitate. After repeating said washing step twice or more, the separated cell precipitate was stored at −20° C. for 12 to 16 hours. After that, the cell precipitate was suspended in 30 in of a lysis buffer (50 mM Tris-HCl, 0.1% SDS, 1 mM DTT, pH 8.0) without forming bubbles, and subjected to ultrasonication on ice using a sonicator equipped with a microtip. The cells were intermittently sonicated for 30 seconds, followed by cooling for 10 seconds, while setting the power to 27% of the maximum power. The total sonication time was 5 minutes. The cell lysates were centrifuged at 4° C., 8,000 rpm for 10 minutes, so as to separate the supernatant and the cell precipitate. The supernatant was loaded onto a Ni-NTA agarose resin where nitrilotriacetic acid agarose was charged with nickel (Ni). The Ni-NTA agarose resin was equilibrated with the lysis buffer. The supernatant was allowed to absorb onto the resin by gently shaking using a rotary shaker for 1 hour or more. The resin absorbed with the inclusion bodies containing the recombinant protein was centrifuged at 4° C., 1,000×g for 5 minutes, to remove the reaction solution and washed with a lysis buffer (50 mM Tris-HCl, 0.1% SDS, 1 mM DTT, pH 8.0) once to remove nonspecific absorbed materials. After washing, the proteins absorbed to the resin were eluted with an elution buffer (containing 250 mM imidazol) with stirring for 1 hour or more at room temperature. The eluted proteins were analyzed with 12% SDS-PAGE gel electrophoresis, stained with Coomassie Brilliant Blue R by gently shaking, and destained with a destaining solution.

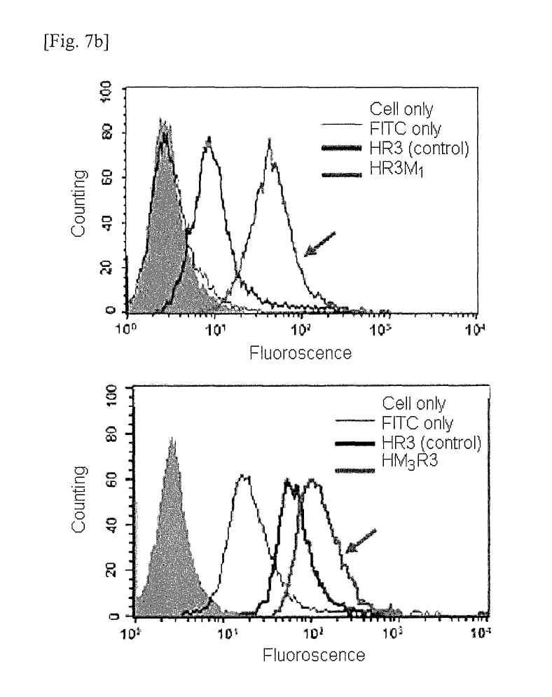

According to the results shown in FIGS. 6a and 6b, all of the cell permeable RUNX3 recombinant proteins fused to kFGF4-derived MTD, JO-57 MTD and JO-85 MTD, respectively, were detected as a single band corresponding to about 47.5 kDa, which confirms that the cell permeable RUNX3 recombinant proteins of the present invention have been purified from the insoluble fraction.

### Example 4

### Cell Permeability Analysis

### <4-1>Flow Cytometry

In order to quantitatively determine the cell permeability of the cell permeable RUNX3 recombinant proteins according to the present invention, flow cytometry was carried out by using the cell permeable RUNX3 recombinant proteins (HM₁R3, HR3M₁, HM₁R3M₁, HM₃R3) on RAW 264.7 cells derived from mouse macrophage, as follows.

The cell permeable RUNX3 recombinant proteins purified in Example 3 above were labeled with FITC (fluorescein-5-isothiocyanate, Molecular Probe). The recombinant protein (2 to 20 mg) was mixed with 1 μl of FITC at a concentration of 333 mg/ml and reacted in a dark room at room temperature for 1 hour with gentle stirring. The reaction solution was subjected to a dialysis against DMEM at 4° C. for 1 day until the unreacted FITC was completely removed, thereby obtaining FITC-conjugated recombinant proteins. Thus obtained FITC-conjugated recombinant proteins were subjected to a Bradford protein assay to measure the protein concentration. As a result, each of the FITC-conjugated recombinant proteins was measured to have a concentration of about 1 μg/μl.

Meanwhile, RAW 264.7 cells were maintained in DMEM supplemented with 10% fetal bovine serum and 5% penicillin/streptomycin (500 mg/ml) and incubated at 37° C. in a humidified atmosphere of 5% CO₂ in air. After the incubation, the cells were treated with 10 μM of each of the FITC-conjugated recombinant proteins prepared above, followed by further culturing them for 1 hour at 37° C. Subsequently, the cells were treated with trypsin/EDTA (T/E) to remove cell surface bound proteins, washed with cold PBS (phosphate buffered saline) three times, and then, subjected to flow cytometry analysis by using a CellQuest Pro software program of the FACS (fluorescence-activated cell sorting) Calibur system (Beckton-Dickinson).

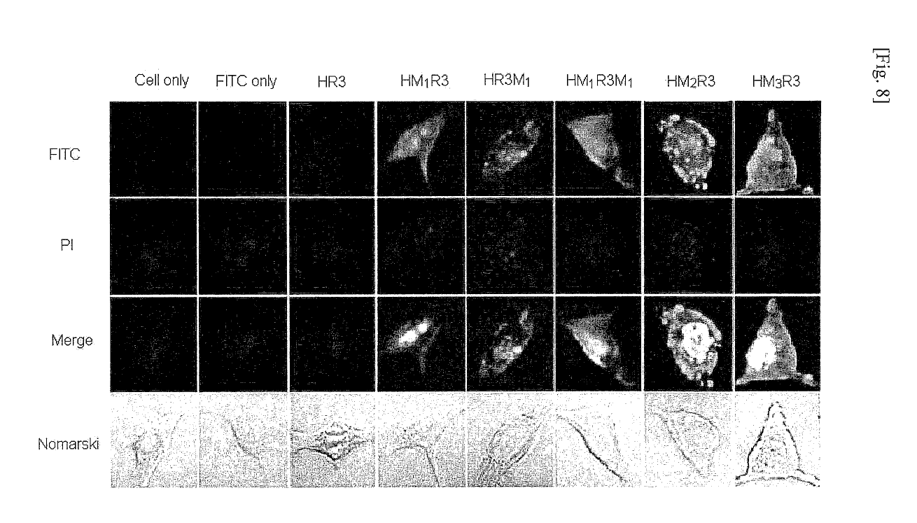

Referring to the results shown in FIGS. 7a and 7b, it was found that in case of the cell permeable RUNX3 recombinant protein to which kFGF4-derived MTD was fused, HM₁R3 containing the MTD fused to its N-terminus and HR3M₁ containing the MTD fused to its C-terminus showed higher cell permeability than HR3 containing no MTD. In case of the cell permeable RUNX3 recombinant protein to which JO-85 MTD was fused, HM₃R3 containing the MTD fused to its N-terminus showed higher cell permeability than HR3 containing no MTD. FIGS. 7a and 7b show the results of the flow cytometry analysis where the gray filled curve represents cell only, the black curve represents FITC only, the blue curve represents the cell permeability of the control protein not fused to a MTD (HR3), each of the red curves represents the cell permeability of the cell permeable recombinant proteins HM₁R3 where MTD1 was fused to its N-terminus, HR3M₁ where MTD₁ was fused to its C-terminus, HM₁R3M₁ MTD₁ was fused to both termini thereof.

### <4-2>Confocal Laser Scanning Microscope Analysis I

To visualize the intracellular localization of human RUNX3 recombinant proteins delivered into a cell, NIH 3T3 cells (Korean Cell Line Bank, Seoul, Republic of Korea) were treated for 1 hour without (cell only) or with FITC (FITC only), or 10 μM FITC-conjugated recombinant proteins lacking kFGF4-derived MTD (HR3) or 10 μM FITC-conjugated recombinant proteins fused to a kFGF4-derived MTD (HM₁R3, HR3M₁, HM₁R3M₁, HM₂R3, HM₃R3), and visualized by confocal laser scanning microscopy. The NIH3T3 cells were maintained in DMEM supplemented with 10% fetal bovine serum, 5% penicillin/streptomycin (500 mg/ml) in 5% CO₂ at 37° C. In order to preserve the FITC fluorescence of the recombinant protein, the glass slide was fixed in 10 μl of a mounting medium for 15 minutes before the observation. For a direct detection of FITC-conjugated recombinant proteins that were internalized, the cells were washed with PBS three times and counterstained with a nuclear fluorescent stain solution, propidium iodide (PI, Sigma-Aldrich, St. Louis, Mo.). The intracellular distribution of the fluorescence was determined at the middle of a single cell analyzed by a confocal laser scanning microscope using a normaski filter.

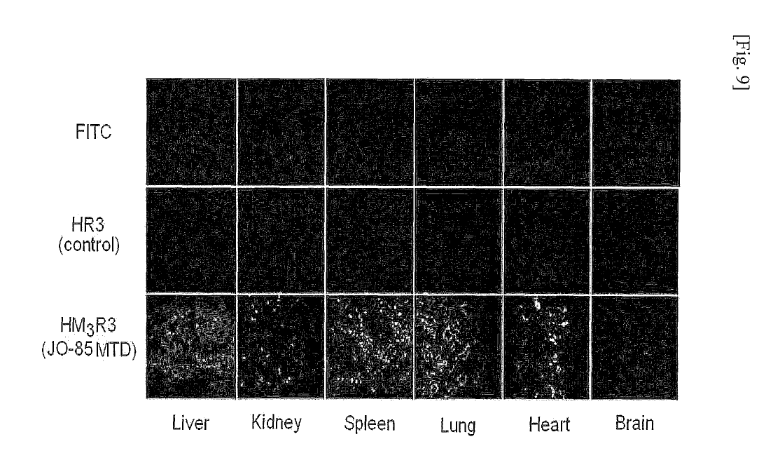

As shown in FIG. 8, it was observed that the cell permeable RUNX3 recombinant proteins stained with FITC (green) and PI (red) were well distributed largely in the nucleus, which is consistent with the cell permeability of the CP-RUNX3 recombinant proteins determined by flow cytometry.

### <4-3>Confocal Laser Scanning Microscope Analysis II

In order to examine whether the cell permeable RUNX3 recombinant proteins according to the present invention exhibit cell permeability with respect to a tissue, the following experiment was performed.

In this experiment, 7-week old Balb/c mice (Central Lab. Animal Inc., Seoul) were used. The mice were administered with 200 μg of the FITC-conjugated RUNX3 recombinant protein (HM₃R3) via intraperitoneal injection. Two hours later, the mice were sacrificed, and various tissue samples were extracted from the liver, kidney, spleen, lung, heart and brain. The extracted tissues were embedded in an OCT compound, freezed, and then sectioned with a microtome to have a thickness of 14 μm. The tissue specimens were mounted on a glass slide and observed with a confocal laser scanning microscope. In order to preserve the FITC fluorescence of the recombinant protein, the glass slide was fixed in 10 μl of a mounting medium for 15 minutes before the observation.

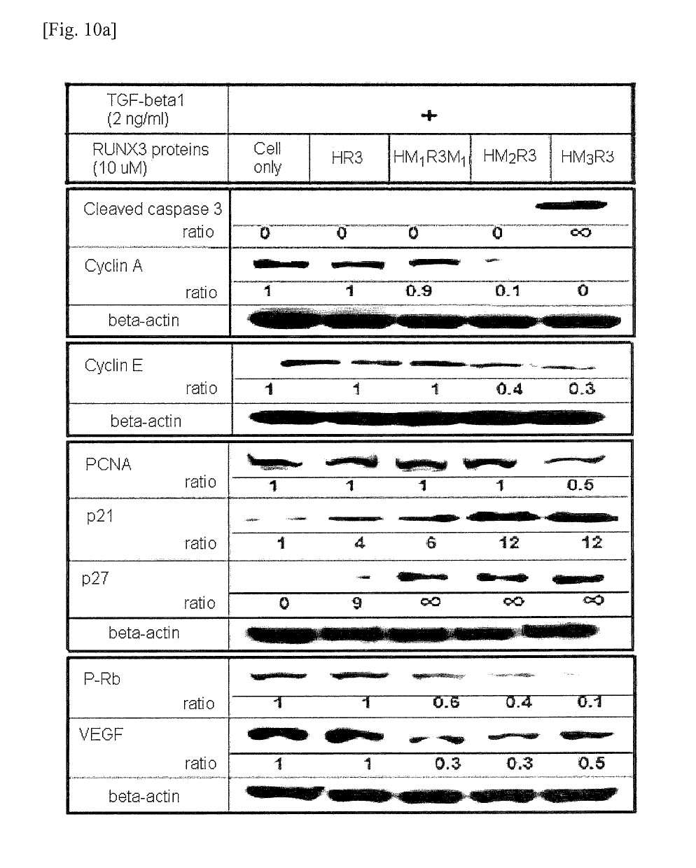

As illustrated in FIG. 9, it was found that protein transport into the nucleus clearly stained with FITC (green) was observed in all of the tissue specimens, which is consistent with the cell permeability of the CP-RUNX3 recombinant proteins determined by flow cytometry.

These results obtained in sections <4-1> to <4-3> of Example 4 above demonstrate that the cell permeable RUNX3 recombinant proteins according to the present invention can be effectively used for transporting a tumor and metastasis suppressor RUNX3 into a target tissue as well as a target cell.

### Example 5

### Cellular Function of Cell Permeable RUNX3 Recombinant Proteins

### <5-1>Western Blotting

In order to confirm the cellular function of the cell permeable RUNX3 recombinant proteins according to the present invention, western blot analysis was carried out on cancer cell lines as follows.

MKN 28 and NCI-N87 cells, gastric cancer cell lines used in this experiment, were purchased from Korean Cell Line Bank (Seoul, Republic of Korea). Each of MKN 28 and NCI-N87 cells was maintained in a RPMI 1640 medium (L-glutamine 300 mg/l, 25 mM HEPES and 25 mM NaHCO₃ 89.3%) supplemented with 9.8% heat inactivated FBS and 1% penicillin/streptomycin in a 5% CO₂ incubator at 37° C. After 2 ml of the RPMI 1640 was added to each well of a 6-well plate, MKN 28 and NCI-N87 cells were inoculated thereto. The well plate was incubated at 37° C. for 1 day, followed by culturing in a serum-free medium, so as to grow the cells in the same cell cycle phase while the cells are adhered to the well plate. After removing the medium, the MKN 28 and NCI-N87 cells adhered to the well plate were washed with cold PBS (phosphate-buffered saline). Subsequently, the cells were treated with each of the cell permeable RUNX3 recombinant proteins HM₁R3M₁, HM₂R3 and HM₃R3 and control protein HR3 at a concentration of 10 μM, and reacted in a 5% CO₂ incubator at 37° C. for 1 hour. After the reaction was completed, the cells were washed twice with PBS, and then, cultured in a 5% CO₂ incubator at 37° C. for 12 hours. After the cultivation was completed, the cells were resuspended in 200 μl of a lysis buffer (20 mM HEPES, pH 7.2, 1% Triton-X, 10% glycerol) and subjected to ultrasonication on ice for 30 minutes, to thereby obtain a cell lysate. The cell lysate was centrifuged at 4° C., 12,000 rpm for 20 minutes to separate the supernatant. The thus obtained supernatant was subjected to a Bradford protein assay to quantitatively measure the protein concentration. The recombinant protein was resuspended in a SDS-PAGE loading buffer at a concentration of 25 μM to prepare a cell lysate sample. The thus prepared cell lysate sample was heated at 90° C. for 5 minutes, and then, stored at −80° C. until use.

For the western blot analysis, p21Wafl/Cipl (21 kDa, Cell Signaling Technology), p27 (27 kDa, Santa Cruz Biotechnology), PCNA (36 kDa, Santa Cruz Biotechnology), cleaved caspase 3 (17/19 kDa, Cell Signaling), cyclin A (54 kDa, Santa Cruz Biotechnology) cyclin E (53 kDa, Santa Cruz Biotechnology), phospho-Rb (Ser807/811, 110 kDa, Santa Cruz Biotechnology) and VEGF (15 kDa, Santa Cruz Biotechnology) were used as primary antibodies, and goat anti-mouse IgG-HRP (Santa Cruz Biotechnology) and goat anti-rabbit IgG-HRP (Santa Cruz Biotechnology) were used as secondary antibodies. The cell lysate sample was applied to a SDS-PAGE at 100 V for 2 hours and transferred onto a polyvinylidene fluoride (PDVF) membrane at 100 V for 1 hour. In order to prevent the nonspecific interaction between the blotted proteins and unrelated antibodies, the PVDF membrane was blocked with 5% non-fat dry milk in TBS/T (10 mM Tris-Cl, pH 8.0, 150 mM NaCl, 0.05% Tween 20) at room temperature for 1 hour. After removing the blocking buffer, the PVDF membrane was washed with TBS/T, followed by incubation with each of the primary antibodies for 1 day at 4° C. After removing the primary antibody solution, the membrane was washed with TBS/T three times, and incubated with the secondary antibody for 1 hour at room temperature. After washing with TBS/T three times, the membrane was stained using an enhanced chemiluminescence (ECL) detection system (GE Healthcare Amersham UK) to visualize the antigen/antibody interaction.

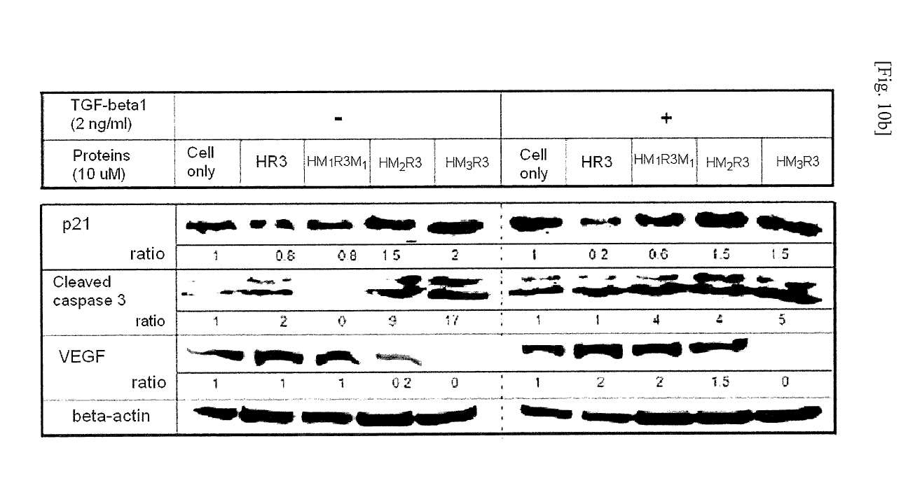

As shown in FIG. 10a, in the MKN 28 cells treated with the cell permeable RUNX3 recombinant protein as compared with cells treated with the control protein, the expression of p21 and p27 that induce cell cycle arrest and caspase 3 that induces apoptosis were enhanced, while the phosphorylation of cyclin A, cyclin E and PCNA and Rb that activate cancer cell cycle and the expression of VEGF that induces metastasis were reduced.

Further, as shown in FIG. 10b, in the NCI-N87 cells treated with the cell permeable RUNX3 recombinant protein as compared with cells treated with the control protein, the expression of p21 that induces cell cycle arrest and caspase 3 that induces apoptosis were enhanced, while the expression of VEGF that induces metastasis were reduced.

In particular, the HM₃R3 recombinant protein where a JO-85 MTD was fused to its N-terminus strongly inhibited the cell cycle of the cultured cancer cells, suggesting that it can be effectively used as a cell cycle inhibitor capable of preventing tumor formation.

### <5-2>Apoptosis-Inducing Effect

In order to examine the cellular function of the cell permeable RUNX3 recombinant proteins according to the present invention, the apoptosis-inducing effect of the recombinant protein was examined by cellular DNA content analysis as follows.

NCI-N87 (Korean Cell Line Bank) cells, a human gastric cancer cell line, were cultured in a RPMI 1640 medium (L-glutamine 300 mg/l, 25 mM HEPES, 25 mM NaHCO₃ 89.3%, heat-inactivated fetal bovine serum 9.8%, streptomycin/penicillin 0.9%) in a 5% CO₂ incubator at 37° C. After 2 ml of the RPMI 1640 medium was added to each well of a 6-well plate, the NCI-N87 cells cultured above were inoculated thereto, and grown at 37° C. for 1 day. Each of the cell permeable recombinant proteins HM₁R3M₁, HM₂R3 and HM₃R3 and control protein HR3 was added to each well at a concentration of 5 μM, followed by culturing them in a serum-free medium for 1 hour. After washing the well plate with cold PBS twice, 2 ml of the RPMI 1640 medium was added to each well, and the well plate was further incubated for 0, 2, 4, and 8 hours, respectively. After that, the cells were washed with cold PBS twice, suspended in 200 μl of PBS, and gently soaked in 4 ml of 70% ethanol. The thus obtained cell suspension was kept on ice for 45 minutes and stored at −20° C. for 1 day. The cell suspension was treated with PI (40 μg/ml) and RNase A (100 μg/ml) and subjected to flow cytometry analysis to quantify the degree of apoptosis induced.

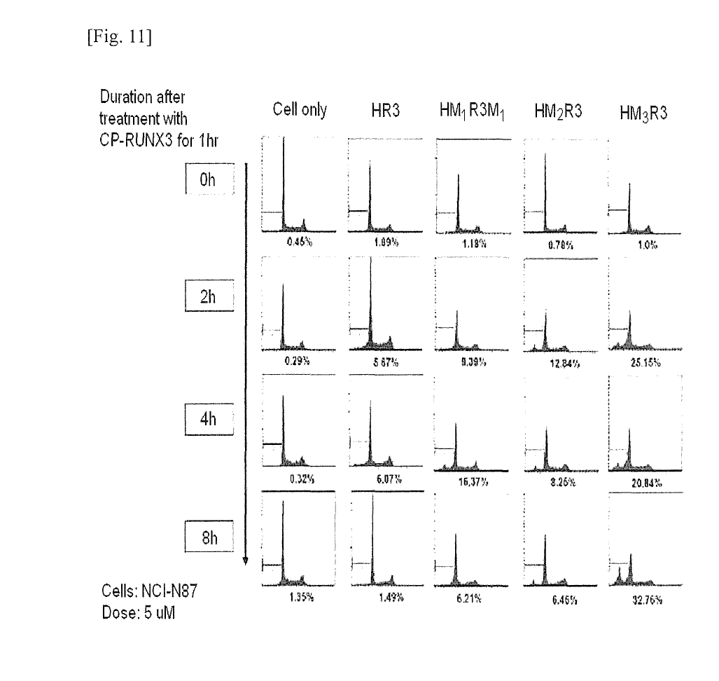

According to the results shown in FIG. 11, it has been found that the cell cycle progression in the cancer cell line was significantly suppressed at a higher rate, and thereby apoptosis was strongly induced in the cells treated with the cell permeable RUNX3 recombinant proteins (HM₁R3M₁, HM₂R3 and HM₃R3) rather than the untreated and control protein (HR3) treated cells. In particular, when the cells were treated with the cell permeable RUNX3 recombinant protein for 8 hours, the highest level of apoptosis was observed, and HM₃R3 to which JO-85 MTD was fused showed the highest apoptosis-inducing effect.

### <5-3>Inhibitory Effect on Cancer Cell Migration

In order to examine the cellular function of the cell permeable RUNX3 recombinant proteins according to the present invention, the inhibitory effect on cancer cell migration of the recombinant protein was examined by a wound healing assay as follows.

MKN 28 and NCI-N87 (Korean Cell Line Bank) cells, human gastric cancer cell lines, were cultured in a RPMI 1640 medium (L-glutamine 300 mg/f, 25 mM HEPES, 25 mM NaHCO₃ 89.3%, heat-inactivated fetal bovine serum 9.8%, streptomycin/penicillin 0.9%) in a 5% CO₂ incubator at 37° C. C. After 2 ml, of the RPMI 1640 medium was added to each well of a 6-well plate, the cells cultured above were inoculated thereto, respectively, and grown at 37° C. for 1 day. Each of the cell permeable recombinant proteins HM₁R3M₁, HM₂R3 and HM₃R3 and control protein HR3 was added to each well at a concentration of 10 μM, followed by culturing them in a serum-free medium for 1 hour. After the cells were washed with PBS twice, they were wounded with a sterile yellow tip, to thereby form a reference line that separated the confluent area from the bare area. To the cells was added 1 ml of a RPMI medium, followed by culturing in a 5% CO₂ incubator at 37° C. for 24 hours. After that, the migration was quantified by counting the number of cells that migrated from the wound edge into the bare area with an inverted light microscope.

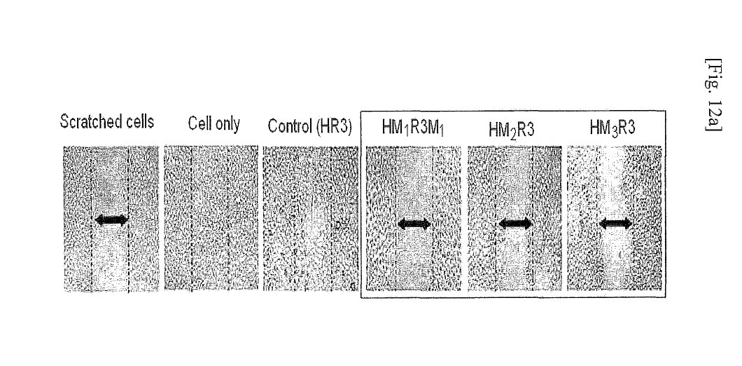

Referring to the results shown in FIG. 12a, the migration of MKN 38 cells was remarkably inhibited in the cells treated with the cell permeable RUNX3 recombinant proteins, HM₁R3M₁ where a kFGF4-derived MTD was fused to its both termini, HM₂R3 where a JO-57 MTD was fused to its N-terminus and HR3M₃ where a JO-85 MTD was fused to its N-terminus, as compared with the control protein.

Further, according to the results shown in FIG. 12b, the migration of NCI-N87 cells was remarkably inhibited in the cells treated with the cell permeable RUNX3 recombinant protein, HM₃R3 where a JO-85 MTD was fused to its N-terminus, as compared with the control protein.

### Example 6

### In Vivo Function of Cell Permeable RUNX3 Recombinant Proteins

### <6-1>Anticancer Effect During Administration

In order to examine the in vivo function of the cell permeable RUNX3 recombinant proteins, the anticancer effect thereof was assessed by using an animal model as follows.

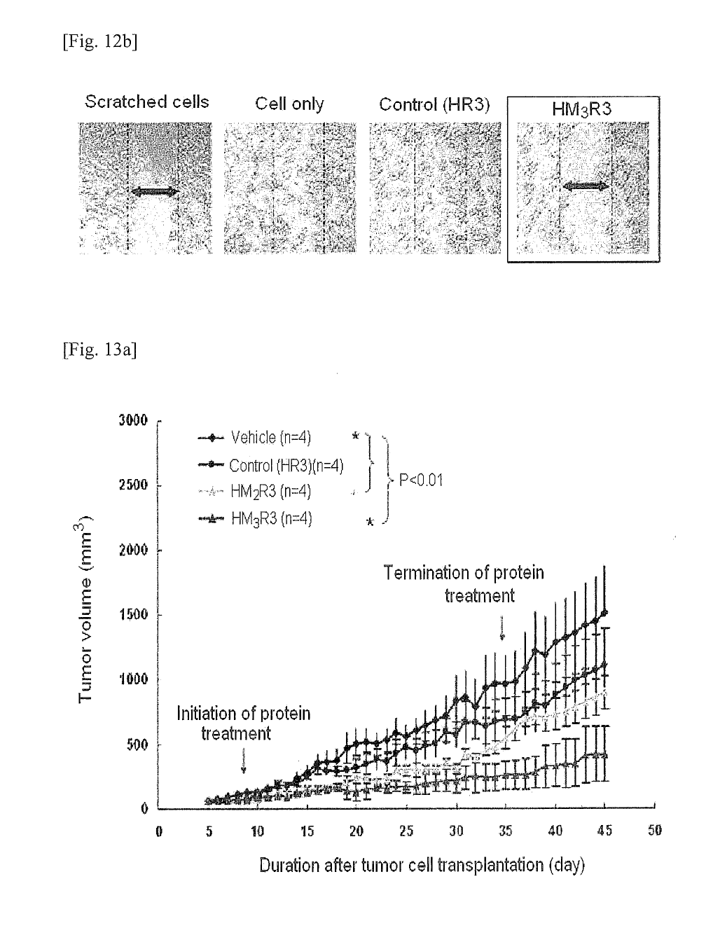

In this experiment, 7-week old Balb/c mice (Central Lab. Animal Inc., Seoul) were used, and sixteen mice were subdivided into 4 groups of 4 mice each. NCI-N87 cells, a human gastric cancer cell line, were administered daily to the right leg of the mouse via subcutaneous injection at a concentration of 1×10⁷ cells by using a syringe (omnican, Germany, B. BRAUN). The mice bearing a tumor of 90 to 100 mm³ in size (width²× length/2) were selected by using a vernier caliper. Each of the cell permeable RUNX3 recombinant proteins HM₂R3 (Group 3, 100 μg) and HM₃R3 (Group 4, 100 μg) was administered daily to the mice at a concentration of 0.5 μg/ml via intraperitoneal injection for 26 days. As a control, 200 μl each of a vehicle (PRMI 1640 medium, Group 1) and MTD-lacking RUNX3 protein HR3 (Group 2) was administered daily to the mice via intraperitoneal injection for 26 days. During the administration for 26 days, the change in tumor size and body weight in the mouse of each group was monitored, and the results are shown in FIGS. 13a and 13b.

According to the results shown in FIGS. 13a and 13b, tumor growth was significantly reduced in the mice treated with each of the cell permeable RUNX3 recombinant proteins HM₂R3 and HM₃R3 (Groups 3 and 4) was significantly reduced compared to that of the control (Groups 1 and 2), and there was no meaningful difference in body weight between the control mice and cell permeable RUNX3 recombinant protein treated mice. The mean value P for the tumor size and body weight in the mice treated with the cell permeable RUNX3 recombinant proteins was less than 0.05, indicating that the results are meaningful.

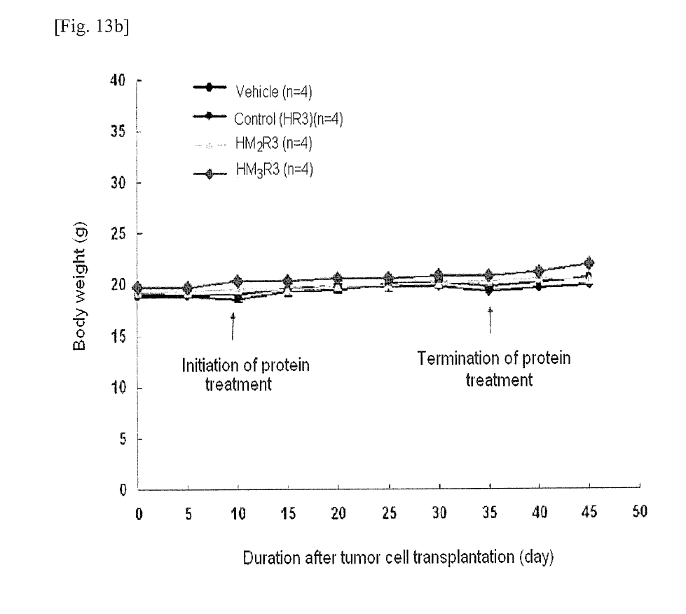

FIG. 14 shows photographs visualizing the change in tumor size and body weight in mice administered with the cell permeable RUNX3 recombinant proteins according to the present invention for 26 days. It was visually observed that the mice treated with the cell permeable RUNX3 recombinant protein showed significantly reduced tumor size than the control mice.

### <6-2>Anticancer Effect After Administration

In order to examine the durability of the in vivo anticancer effect of the cell permeable RUNX3 recombinant proteins (HM₂R3 and HM₃R3) after administration, each of the recombinant proteins was administered to the mice for 26 days according to the same method as described in section <6-1> of Example 7 above. After the administration was terminated, 2 mice were selected from each group, and their tumor size was observed for 7 days.

According to the results shown in FIGS. 13a and 13b, the tumor size was increased in all of the experimental groups. In particular, while the tumor size was remarkably increased in the HM₂R3 treated mice (Group 3) that showed significantly reduced tumor size during the administration, as similar to the control, the HM₃R3 treated mice (Group 4) showed a significantly smaller increase in tumor size. These results suggest that the cell permeable RUNX3 recombinant protein HM₃R3 can stably maintain its anticancer effect for a prolonged period, and thus can be effectively used as a cell cycle inhibitor in cancer cells.

### Example 7

### Immunohistochemical Analysis after Administration of Cell Permeable RUNX3 Recombinant Proteins

In order to examine the effect of inducing apoptosis in tumor tissues after the administration of the cell permeable RUNX3 recombinant proteins, an immunohistological analysis was performed on the same mouse model as used in Example 6.

In particular, the cell permeable RUNX3 recombinant proteins (HM₂R3 and HM₃R3), vehicle, and HR3 (control) were administered to the mice subdivided into four groups (4 mice per group) via subcutaneous injection for 26 days, respectively, according to the same method as described in Example 6. After that, the mice had undergone further observation for 5 days after the administration was terminated, and then, organ and tumor tissue samples were extracted therefrom. Each of the organ and tumor tissue samples was fixed in formalin and embedded in paraffin melted at 62° C. in an embedding center, to thereby prepare a paraffin block. The paraffin block was sliced with a microtome to have a thickness of 5 μm, where the slices were mounted on a slide glass and treated with xylene for 5 minutes three times to remove paraffin. Next, the glass slide was hydrated by successively treating with 100%, 100%, 95%, 70% and 50% ethanol each for 3 minutes, washed with water for 5 minutes. In order to induce antigen presentation from the tissue, the glass slide was trated with 0.05% trypsin/EDTA and stored at 37 t for 20 minutes. The glass slide was then washed with water for 5 minutes, treated with 1% hydrogen peroxide for 10 minutes, washed with water three times each for 5 minutes, and then, washed with TBS (Tris buffered saline) for 5 minutes. For blocking non-specific antigen binding, the glass slide was treated with a normal horse serum for 1 hour. The slide glass was incubated with p21 Wafl/Cipl (21 kDa, Cell Signaling Technology) and VEGF (15 kDa, Santa Cruz Biotechnology) as primary antibodies at 4° C. for 1 day, followed by washing with TBS buffer three times each for 5 minutes. The slide glass was incubated with the goat anti-mouse IgG-HRP (Santa Cruz Biotechnology) and a gaot anti-rabbit IgG-HRP (Santa Cruz Biotechnology) as secondary antibodies for 1 hour at room temperature, followed by staining with a DAB (diaminobenzidine tetrahydrochloride, Vector Laboratories, Inc) substrate for 2 to 3 minutes. Subsequently, the slide glass was washed with distilled water and subjected to counter-staining with hematoxylin. Finally, the glass slide was dehydrated by successively treating with 95%, 95%, 100%, and 100% ethanol each for 10 seconds and dewaxed by treating with xylene twice each for 10 seconds. And then, the glass slide was sealed with Canada balsam as a mounting medium and observed with an optical microscope.

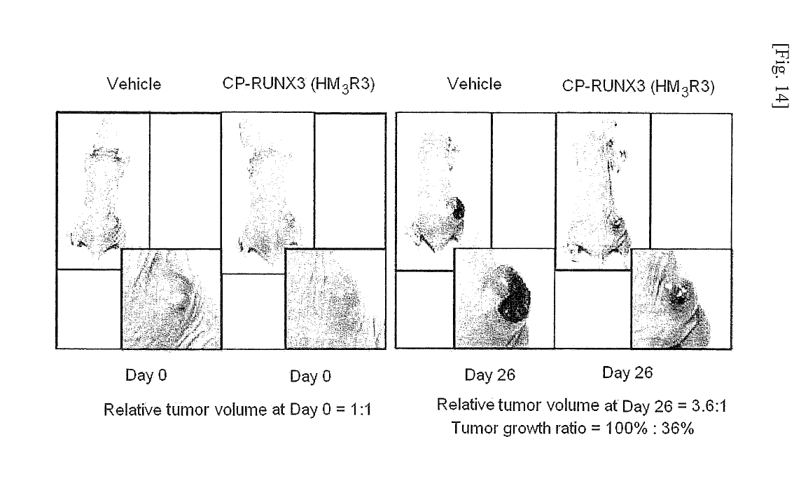

Referring to the results shown in FIG. 15, it was confirmed that in the lung and tumor tissue samples treated with the cell permeable RUNX3 recombinant protein (HM₃R3) as compared with those treated with the vehicle and control protein, the expression of p21 that induces cell cycle arrest was enhanced, while the expression of VEGF that induces metastasis was reduced.

### Example 8

### Apoptosis-Inducing Effect after the Administration of Cell Permeable RUNX3 Recombinant Proteins I

In order to examine the effect of inducing apoptosis in tumor tissues after the administration of the cell permeable RUNX3 recombinant proteins, a TUNEL (terminal deoxynucleotidyl transferase-mediated dUTP nick-end labeling) assay was performed by using the same mouse model as described in Example 8.

In particular, the cell permeable RUNX3 recombinant proteins (HM₂R3 and HM₃R3), vehicle, and HR3 (control) were administered to the mice subdivided into four groups (4 mice per group) via subcutaneous injection for 26 days, respectively, according to the same method as described in Example 6. After that, the mice had undergone further observation for 5 days after the administration was terminated, and then, a tumor tissue sample was extracted therefrom. The glass slide was prepared by using the extracted tumor tissue sample according to the same method as described in Example 7. The glass slide was treated with xylene for 5 minutes twice, to thereby remove paraffin. It was then successively treated with 100% ethanol twice for 5 minutes, and 90%, 80% and 70% ethanol each for 3 minutes so as to dehydrate the tumor tissue, followed by incubation in PBS for 5 minutes. The glass slide was treated with 0.1% Trition® X-100 dissolved in a 0.1% sodium citrate solution for 8 minutes, and washed with PBS twice for 2 minutes. After a drop of TUNEL reaction buffer (50 μl, Roche, USA) was added to the glass slide, the glass slide was incubated in a humidified incubator at 37° C. for 1 hour, washed with PBS three times, and then, observed with a fluorescence microscope.

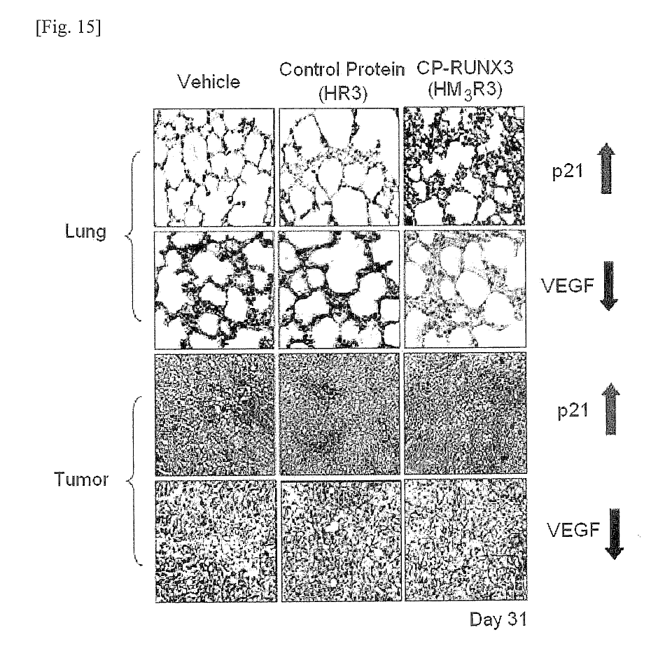

Referring to the results shown in FIG. 16, there was no significant histological change in tumor tissue extracted from the mice treated with the vehicle and control protein (HR3), while in the tumor tissue extracted from the mice treated with the cell permeable RUNX3 recombinant protein (HM₃R3), a region stained in red representing the characteristic of apoptosis was observed, confirming the effect of inducing apoptosis of the cell permeable RUNX3 recombinant protein according to the present invention. Further, it was also observed that in the mice treated with the cell permeable RUNX3 recombinant protein according to the present invention, apoptosis was still induced in cancer cells after the administration was terminated.

### Example 9

### Apoptosis-Inducing Effect after the Administration of Cell Permeable RUNX3 Recombinant Proteins I

In order to examine the effect of inducing apoptosis in tumor tissues after the administration of the cell permeable RUNX3 recombinant proteins, the following histochemical assay was performed by using an ApopTag Peroxidase in situ Apoptosis Detection Kit (Chemicon, S7100).

In particular, the cell permeable RUNX3 recombinant proteins (HM₂R3 and HM₃R3), vehicle, and HR3 (control) were administered to the mice subdivided into four groups (4 mice per group) via subcutaneous injection for 26 days, respectively, according to the same method as described in Example 6. After that, the mice had undergone further observation for 5 days after the administration was terminated, and then, a tumor tissue sample was extracted therefrom. The glass slide was prepared by using the extracted tumor tissue sample according to the same method as described in Example 7. The glass slide was treated with xylene for 5 minutes twice, to thereby remove paraffin. It was then successively treated with 100% ethanol twice for 5 minutes, and 90%, 80% and 70% ethanol each for 3 minutes so as to dehydrate the tumor tissue, followed by incubation in PBS for 5 minutes. The glass slide was treated with 20 μg/ml, of proteinase K (Sigma) for 15 minutes, washed with distilled water, and then, treated with 3% H₂O₂ (vol/vol, in PBS) for 5 minutes, to thereby inhibit the activity of endogenous peroxidase. The glass slide was treated with an equilibration buffer for 10 seconds, followed by treating with a terminus dexoynucleotidyl transferase (TdT) at 37° C. for 1 hour. After the reaction was completed, the glass slide was treated with a stop buffer and washed. Next, the glass slide was treated with a DAB coloring agent for 5 minutes, and counterstained with methyl green. After the staining, the glass slide was dehydrated, sealed with a cover slip, and observed with an optical microscope.

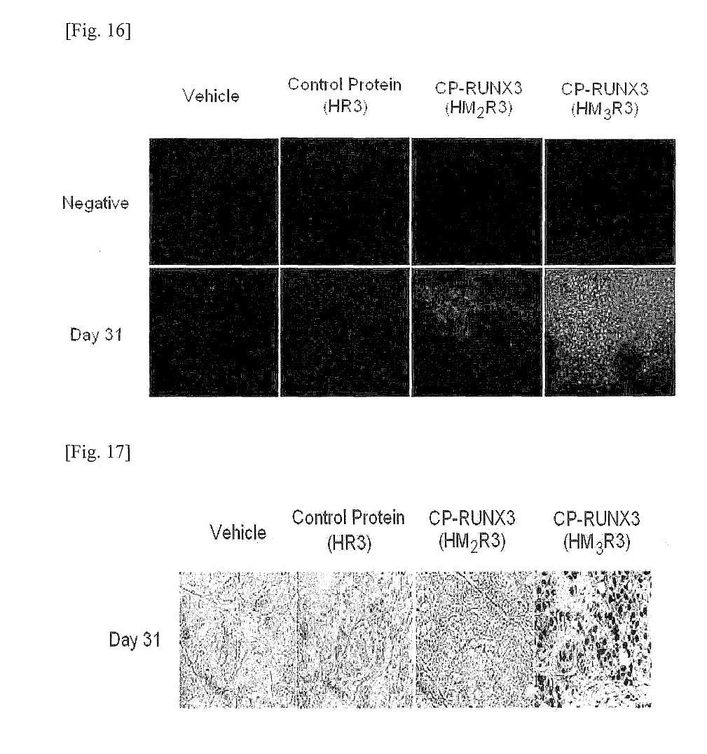

According to the results shown in FIG. 17, there was no significant histological change in tumor tissue extracted from the mice treated with the vehicle and control protein (HR3), while in the mouse tumor tissues treated with the cell permeable RUNX3 recombinant protein (HM₃R3), a region stained in brown representing the characteristic of apoptosis was observed, confirming the effect of inducing apoptosis of the cell permeable RUNX3 recombinant protein according to the present invention. Further, it was also observed that in the mice treated with the cell permeable RUNX3 recombinant protein according to the present invention, apoptosis is still induced in cancer cells after the administration was terminated.

### Example 10

### Comparison of Protein Expression Pattern after the Administration of Cell Permeable RUNX3 Recombinant Proteins

In order to examine the change in protein expression pattern in the tumor tissue treated with the cell permeable RUNX3 recombinant protein according to the present invention, a microarray assay was performed as follows.

In particular, each of the cell permeable RUNX3 recombinant protein (HM₃R3), vehicle and HR3 (control) was administered to the mice subdivided into four groups via subcutaneous injection for 26 days, and then left alone for 5 days after the administration was terminated, according to the same method as described in Example 6 above. Thirty one days after the administration was initiated, tumor tissue samples were extracted from the mouse of each group and freezed with liquid nitrogen. Total RNA was isolated from the tumor tissue by using a TRIZOL reagent (Invitrogen) according to the manufacturer's instruction, and treated with an RNase-free DNase (Life Technologies, Inc.), to thereby completely remove the remaining genomic DNA.

The thus isolated RNA was subjected to synthesis and hybridization of a target cRNA probe by using a Low RNA Input Linear Amplification kit (Agilent Technology) according to the manufacturer's instruction. In brief, 1 μg of total RNA was mixed with a T7 promoter specific primer and reacted at 65° C. for 10 minutes. A cDNA master mix was prepared by mixing a first strand buffer (5×), 0.1 M DTT, 10 mM dNTP mix, RNase-Out and MMLV-RT (reverse transcriptase), and added to the reaction mixture. The resulting mixture was reacted at 40° C. for 2 hours, followed by reacting at 65° C. for 15 minutes, to thereby terminate the reverse transcription and dsDNA synthesis. A transcription master mix was prepared by mixing a transcription buffer (4×), 0.1 M DTT, NTP mix, 50% PEG, RNase-Out, inorganic pyrophosphatase, T7-RNA polymerase and cyanine (3/5-CTP) according to the manufacturer's instruction. The thus prepared transcription master mix was added to the dsDNA reaction mixture and reacted at 40° C. for 2 hours so as to perform dsDNA transcription. The thus amplified and labeled cRNA was purified with a cRNA Cleanup Module (Agilent Technology) according to the manufacturer's instruction. The labeled target cRNA was quantified by using a ND-1000 spectrophotometer (Nanoprop Technologies, Inc.). After the labeling efficiency was examined, cRNA was mixed with a blocking agent (10×) and a fragmentation buffer (25×), and reacted at 60° C. for 30 minutes so as to carry out the fragmentation of cRNA. The fragmented cRNA was resuspended in a hybridization buffer (2×) and directly dropped on a Whole Human Genome Oligo Microarray (44K). The microarray was subjected to hybridization in a hybridization oven (Agilent Technology) at 65° C. for 17 hours, followed by washing according to the manufacturer's instruction (Agilent Technology).

The hybridization pattern was read by using a DNA microarray scanner (Agilent Technology) and quantified by using a Feature Extraction Software (Agilent Technology). Data normalization and selection of fold-changed genes were carried out by using a Gene Spring GX 7.3 soft wear (Agilent Technology). The average of the normalized ratio was calculated by dividing a normalized signal channel strength by a normalized control channel strength. Functional annotation for a gene was conducted by using a Gene Spring GX 7.3 software (Agilent Technology) according to the Gene Ontology™ Consortium (http://www.geneontology.org/index.shtml).

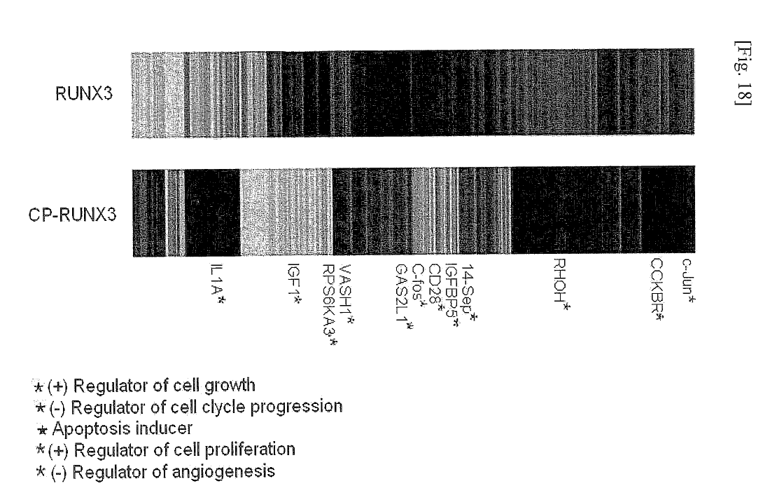

The results of the microarray analysis are summarized in FIG. 18 and Tables 3 to 8, where Table 3 shows the expression pattern of apoptosis-relating genes, Table 4 shows that of cell adhesion-relating genes, Table 5 shows that of cell cycle-relating genes, Table 6 shows that of cell growth-relating genes, Table 7 shows that of cell proliferation-relating genes, and Table 8 shows that of defence immunity-relating genes.

TABLE 3

| | | Expression pattern | | Total | |
|---|---|---|---|---|---|
| | | Veh. vs | Veh. vs | relative | |
| Gene | Genbank ID | HR3 | CP-HR3 | ratio | t-test/p-value |
| CD28 | NM_006139 | 1.27 | 0.17 | 0.13 | 0.35/0.05 |
| molecule | | | | | |
| BCL2L13 | NM_015367 | 0.86 | 0.32 | 0.37 | 0.27/0.01 |
| TAOK2 | NM_004783 | 1.72 | 0.28 | 0.16 | 0.04/0.01 |
| PPP2R1B | NM_002716 | 2.88 | 0.56 | 0.19 | 0.03/0.18 |
| PHF17 | AK127326 | 2.03 | 0.98 | 0.48 | 0.02/0.86 |
| IL1A | NM_000575 | 0.40 | 1.14 | 2.85 | 0.00/0.39 |
| SEMA6A | NM_020796 | 0.50 | 1.22 | 2.44 | 0.03/0.23 |
| PIK3R2 | NM_005027 | 0.22 | 0.65 | 2.95 | 0.01/0.05 |

TABLE 4

| | | Expression | | | |
|---|---|---|---|---|---|
| | | pattern | | | |
| | | | Veh. vs | Total | |
| | | Veh. vs | CP- | relative | t-test/ |
| Gene | Genbank ID | HR3 | HR3 | ratio | p-value |
| PPP2R1B | NM_002716 | 2.88 | 0.56 | 0.19 | 0.03/0.18 |
| CX3CL1 | NM_002996 | 1.73 | 0.73 | 0.42 | 0.04/0.09 |
| CNTN6 | NM_014461 | 1.95 | 0.59 | 0.30 | 0.04/0.08 |
| ADAM15 | NM_207191 | 1.67 | 0.83 | 0.50 | 0.04/0.22 |
| MUC4 | NM_018406 | 2.39 | 0.81 | 0.34 | 0.02/0.18 |
| ARHGDIG | NM_001176 | 2.43 | 0.32 | 0.13 | 0.02/0.01 |
| TAOK2 | NM_004783 | 1.72 | 0.28 | 0.16 | 0.04/0.01 |
| TGM2 | NM_004613 | 4.65 | 1.64 | 0.35 | 0.00/0.09 |
| FN1 | NM_054034 | 2.51 | 1.01 | 0.40 | 0.02/0.98 |
| JAM2 | NM_021219 | 0.91 | 0.31 | 0.34 | 0.57/0.02 |
| DLG5 | NM_004747 | 0.94 | 0.33 | 0.35 | 0.57/0.01 |
| OPCML | NM_001012393 | 1.03 | 0.15 | 0.15 | 0.79/0.00 |
| COL11A1 | NM_080629 | 0.73 | 0.27 | 0.37 | 0.09/0.01 |

TABLE 5

| | | Expression pattern | | Total | |
|---|---|---|---|---|---|
| | | Veh. vs | Veh. vs | relative | |
| Gene | Genbank ID | HR3 | CP-HR3 | ratio | t-test/p-value |
| PPP2R1B | NM_002716 | 2.88 | 0.56 | 0.19 | 0.03/0.18 |
| PTN | NM_002825 | 0.41 | 0.20 | 0.49 | 0.02/0.01 |
| DLG5 | NM_004747 | 0.94 | 0.33 | 0.35 | 0.57/0.01 |
| IL1A | NM_000575 | 0.40 | 1.14 | 2.85 | 0.00/0.39 |
| 14-Sep | NM_207366 | 1.47 | 3.07 | 2.09 | 0.10/0.02 |
| LTBP2 | NM_000428 | 1.00 | 2.64 | 2.64 | 0.99/0.02 |
| GAS2L1 | NM_152236 | 1.07 | 2.72 | 2.54 | 0.57/0.01 |
| VASH1 | NM_014909 | 0.76 | 1.84 | 2.42 | 0.51/0.04 |

TABLE 6

| | | Expression | | | |
|---|---|---|---|---|---|
| | | pattern | | | |
| | | | Veh. vs | Total | |
| | | Veh. vs | CP- | relative | t-test/ |
| Gene | Genbank ID | HR3 | HR3 | ratio | p-value |
| SGCG | NM_000231 | 3.70 | 1.43 | 0.39 | 0.02/0.24 |
| DVL1 | NM_181870 | 5.13 | 1.49 | 0.29 | 0.01/0.13 |
| SECTM1 | NM_003004 | 3.52 | 1.17 | 0.33 | 0.01/0.27 |
| COL1A2 | NM_000089 | 3.99 | 1.17 | 0.29 | 0.01/0.27 |
| TLX2 | NM_016170 | 4.55 | 2.20 | 0.48 | 0.01/0.02 |
| TLX3 | NM_021025 | 2.19 | 0.56 | 0.26 | 0.02/0.33 |
| CNTN6 | NM_014461 | 1.95 | 0.59 | 0.30 | 0.04/0.08 |
| PAX1 | NM_006192 | 1.82 | 0.54 | 0.30 | 0.64/0.02 |
| PPP2R1B | NM_002716 | 2.88 | 0.56 | 0.19 | 0.03/0.18 |
| MLLT6 | NM_005937 | 3.01 | 0.57 | 0.19 | 0.02/0.12 |
| TGM5 | NM_201631 | 2.22 | 0.92 | 0.41 | 0.02/0.72 |
| PHF17 | AK127326 | 2.03 | 0.98 | 0.48 | 0.02/0.86 |
| ECE2 | NM_014693 | 1.91 | 0.86 | 0.45 | 0.03/0.31 |
| JUN | NM_002228 | 1.92 | 0.89 | 0.46 | 0.00/0.01 |
| COL1A2 | NM_000089 | 1.79 | 0.84 | 0.47 | 0.00/0.00 |
| CSF3 | NM_000759 | 1.47 | 0.39 | 0.27 | 0.12/0.02 |
| TAOK2 | NM_004783 | 1.72 | 0.28 | 0.16 | 0.04/0.01 |
| DACH1 | NM_080759 | 1.11 | 0.32 | 0.29 | 0.46/0.02 |
| FOS | NM_005252 | 1.17 | 0.47 | 0.40 | 0.27/0.02 |
| PLXNA1 | NM_032242 | 0.89 | 0.43 | 0.48 | 0.55/0.02 |
| IGF1 | NM_000618 | 0.84 | 0.38 | 0.45 | 0.23/0.02 |
| IGFBP5 | NM_000599 | 0.95 | 0.46 | 0.48 | 0.67/0.02 |
| RPS6KA3 | NM_004586 | 1.01 | 0.41 | 0.41 | 0.95/0.02 |
| MYL1 | NM_079422 | 0.78 | 2.30 | 2.95 | 0.39/0.05 |
| KRT14 | NM_000526 | 0.91 | 2.01 | 2.21 | 0.44/0.02 |
| SLC25A25 | NM_001006641 | 0.61 | 2.76 | 4.5 | 0.07/0.01 |
| FBXW4 | NM_022039 | 0.78 | 1.65 | 2.12 | 0.14/0.04 |
| FLOT2 | NM_004475 | 1.07 | 2.46 | 2.30 | 0.68/0.02 |
| APOL2 | NM_145637 | 1.18 | 3.49 | 2.96 | 0.24/0.01 |
| NPR3 | NM_000908 | 0.30 | 0.78 | 2.6 | 0.04/0.21 |
| SEMA6A | NM_020796 | 0.50 | 1.22 | 2.44 | 0.03/0.23 |

TABLE 7

| | | Expression pattern | | Total | |
|---|---|---|---|---|---|
| | | Veh. vs | Veh. vs | relative | |
| Gene | Genbank ID | HR3 | CP-HR3 | ratio | t-test/p-value |
| CD28 | NM_006139 | 1.27 | 0.17 | 0.13 | 0.35/0.05 |
| CSF3 | NM_000759 | 1.47 | 0.39 | 0.27 | 0.12/0.02 |
| LGI1 | NM_005097 | 0.45 | 0.16 | 0.36 | 0.04/0.02 |
| PTN | NM_002825 | 0.41 | 0.20 | 0.49 | 0.02/0.01 |
| DLG5 | NM_004747 | 0.94 | 0.33 | 0.35 | 0.57/0.01 |
| IGF1 | NM_000618 | 0.84 | 0.38 | 0.45 | 0.23/0.02 |
| IL1A | NM_000575 | 0.40 | 1.14 | 2.85 | 0.00/0.39 |
| CCKBR | NM_176875 | 2.26 | 0.93 | 0.41 | 0.02/0.65 |
| ARTN | NM_057091 | 4.34 | 1.88 | 0.43 | 0.01/0.03 |

TABLE 8

| | | Expression | | | |
|---|---|---|---|---|---|
| | | pattern | | | |
| | | | Veh. vs | Total | |
| | | Veh. vs | CP- | relative | t-test/ |
| Gene | Genbank ID | HR3 | HR3 | ratio | p-value |
| LILRB4 | NM_006847 | 1.45 | 3.05 | 2.10 | 0.27/0.02 |
| SNRP70 | NM_003089 | 1.22 | 2.59 | 2.12 | 0.20/0.02 |
| PAGE2B | NM_001015038 | 0.80 | 1.13 | 1.41 | 0.05/0.39 |
| NCR3 | NM_147130 | 0.90 | 1.47 | 1.63 | 0.01/0.07 |
| CCDC34 | NM_030771 | 0.55 | 1.29 | 2.35 | 0.01/0.13 |
| HLA-DOA | NM_002119 | 3.47 | 1.08 | 0.31 | 0.02/0.79 |
| HLA-DPB1 | NM_002121 | 3.15 | 0.99 | 0.31 | 0.02/0.94 |
| PPP2R1B | NM_002716 | 3.44 | 0.56 | 0.16 | 0.03/0.18 |
| HLA-DRA | NM_019111 | 2.69 | 0.75 | 0.28 | 0.02/0.13 |
| CD28 | NM_006139 | 1.27 | 0.17 | 0.13 | 0.35/0.05 |
| CSF3 | NM_000759 | 1.47 | 0.39 | 0.27 | 0.12/0.02 |
| GAGE7 | NM_021123 | 1.27 | 0.57 | 0.45 | 0.15/0.03 |
| SEMA3E | NM_012431 | 0.75 | 0.23 | 0.31 | 0.44/0.04 |

As described in Table 3 above, in case of the apoptosis-relating genes, the expressions of interleukin α (IL 1A) and semaphorin 6A (SEMA6A) were up-regulated by about 2.0-fold or more in the mouse group treated with the cell permeable RUNX3 recombinant protein compared to that treated with the control protein.

As described in Table 4 above, in case of the cell adhesion-relating genes, the expressions of protein phosphatase 2 regulatory (PPP2R1B), RhoGDP dissociation inhibitor Γ (ARHGHIG) and opioid binding protein (OPCML) were down-regulated by about 2.0-fold or more in the mouse group treated with the cell permeable RUNX3 recombinant protein compared to that treated with the control protein.

As described in Table 5 above, in case of the cell cycle regulation-relating genes, while the expressions of protein phosphatase 2 regulatory (PPP2R1B) and pleiotrophin (PTN) were down-regulated by about 2.0-fold or more, the expressions of GAS2L1 (growth arrest-specific 2 like 1) and VASH1 (vasohibin 1) were up-regulated by about 2.0-fold or more in the mouse group treated with the cell permeable RUNX3 recombinant protein compared to that treated with the control protein.

As described in Table 6 above, in case of the cell growth-relating genes, the expressions of c-JUN, insulin-like growth factor (IGF1), ribosomal protein S6 kinase (RPS6KA3) and CD28 were down-regulated by about 2.0-fold or more in the mouse group treated with the cell permeable RUNX3 recombinant protein compared to that treated with the control protein.

As described in Table 7 above, in case of the cell proliferation-relating genes, the expressions of CD28 and cholecystokinin-B/gastrin receptor were down-regulated by about 2.0-fold or more in the mouse group treated with the cell permeable RUNX3 recombinant protein compared to that treated with the control protein.

As described in Table 8 above, in case of defense immunity-relating genes, while the expressions of leukocyte immunoglobulin-like receptor (LILRB4) and CCDC34 (coiled-coil domain containing 34) were up-regulated by about 2.0-fold or more, the expression of CD28 was down-regulated by about 2.0-fold or more in the mouse group treated with the cell permeable RUNX3 recombinant protein compared to that treated with the control protein.

Although the invention has been described in detail for the purpose of illustration, it is understood that such detail is solely for that purpose, and variations can be made therein by those skilled in the art without departing from the spirit and scope of the invention which is defined by the following claims.

1. A cell permeable RUNX3 recombinant protein comprising a macromolecule transduction domain (MTD) and a human tumor and metastasis suppressor RUNX3, said MTD being fused to N-terminus, C-terminus, or both termini of the human tumor and metastasis suppressor RUNX3.

2. The cell permeable RUNX3 recombinant protein according to claim 1, wherein the human tumor and metastasis suppressor RUNX3 is in a full-length form having an amino acid sequence represented by SEQ ID NO: 2 which includes all of N-terminal, R-terminal and PST-rich domains, or a truncated form lacking one or more of the N-terminal, R-terminal and PST-rich domains.

3. The cell permeable RUNX3 recombinant protein according to claim 1, wherein the MTD has an amino acid sequence selected from the group consisting of SEQ ID NOS: 3 to 196.

4. The cell permeable RUNX3 recombinant protein according to claim 3, wherein the MTD is selected from the group consisting of a kFGF4 (kaposi fibroblast growth factor 4)-derived MTD having an amino acid sequence represented by SEQ ID NO: 3, a JO-13 MTD having an amino acid sequence represented by SEQ ID NO: 16, a JO-57 MTD having an amino acid sequence represented by SEQ ID NO: 60, a JO-85 MTD having an amino acid sequence represented by SEQ ID NO: 88, and a JO-108 MTD having an amino acid sequence represented by SEQ ID NO: 111.

5. The cell permeable RUNX3 recombinant protein according to claim 1, further comprising:
a nuclear localization sequence (NLS) and a histidine-tag affinity domain, said nuclear localization sequence and histidine-tag affinity domain being covalently coupled to one end of the recombinant protein.

6. The cell permeable RUNX3 recombinant protein according to any one of claims 1 to 5, wherein the recombinant protein is selected from the group consisting of:
a recombinant protein wherein a kFGF4-derived MTD having an amino acid sequence represented by SEQ ID NO: 3 is fused to the N-terminus of a full-length RUNX3 having an amino acid sequence represented by SEQ ID NO: 2;
a recombinant protein wherein a kFGF4-derived MTD having an amino acid sequence represented by SEQ ID NO: 3 is fused to the C-terminus of a full-length RUNX3 having an amino acid sequence represented by SEQ ID NO: 2;
a recombinant protein wherein a kFGF4-derived MTD having an amino acid sequence represented by SEQ ID NO: 3 is fused to both termini of a full-length RUNX3 having an amino acid sequence represented by SEQ ID NO: 2;
a recombinant protein wherein a kFGF4-derived MTD having an amino acid sequence represented by SEQ ID NO: 3 is fused to the C-terminus of a RUNX3 N-terminal domain fragment lacking R-terminal and PST-rich domains in the amino acid sequence represented by SEQ ID NO: 2;
a recombinant protein wherein a kFGF4-derived MTD having an amino acid sequence represented by SEQ ID NO: 3 is fused to the C-terminus of a RUNX3 R-terminal domain fragment lacking N-terminal and PST-rich domains in the amino acid sequence represented by SEQ ID NO: 2;
a recombinant protein wherein a kFGF4-derived MTD having an amino acid sequence represented by SEQ ID NO: 3 is fused to the C-terminus of a RUNX3 PST-rich domain fragment lacking N- and R-terminal domains in the amino acid sequence represented by SEQ ID NO: 2;
a recombinant protein wherein a kFGF4-derived MTD having an amino acid sequence represented by SEQ ID NO: 3 is fused to the C-terminus of a RUNX3 N- and R-terminal domain fragment lacking a PST-rich domain in the amino acid sequence represented by SEQ ID NO: 2;
a recombinant protein wherein a kFGF4-derived MTD having an amino acid sequence represented by SEQ ID NO: 3 is fused to the C-terminus of a RUNX3 R-terminal and PST-rich domain fragment lacking an N-terminal domain in the amino acid sequence represented by SEQ ID NO: 2;
a recombinant protein wherein a kFGF4-derived MTD having an amino acid sequence represented by SEQ ID NO: 3 is fused to the C-terminus of a portion of a RUNX3 R-terminal and PST-rich domain fragment lacking an N-terminal domain which corresponds to amino acid residues 68-200 in the amino acid sequence of SEQ ID NO: 2;
a recombinant protein wherein a JO-57 MTD having an amino acid sequence represented by SEQ ID NO: 60 is fused to the N-terminus of a full-length RUNX3 having an amino acid sequence represented by SEQ ID NO: 2;
a recombinant protein wherein a JO-57 MTD having an amino acid sequence represented by SEQ ID NO: 60 is fused to the C-terminus of a full-length RUNX3 having an amino acid sequence represented by SEQ ID NO: 2;
a recombinant protein wherein a JO-57 MTD having an amino acid sequence represented by SEQ ID NO: 60 is fused to both termini of a full-length RUNX3 having an amino acid sequence represented by SEQ ID NO: 2;
a recombinant protein wherein a JO-85 MTD having an amino acid sequence represented by SEQ ID NO: 88 is fused to the N-terminus of a full-length RUNX3 having an amino acid sequence represented by SEQ ID NO: 2;
a recombinant protein wherein a JO-85 MTD having an amino acid sequence represented by SEQ ID NO: 88 is fused to the C-terminus of a full-length RUNX3 having an amino acid sequence represented by SEQ ID NO: 2;
a recombinant protein wherein a JO-85 MTD having an amino acid sequence represented by SEQ ID NO: 88 is fused to both termini of a full-length RUNX3 having an amino acid sequence represented by SEQ ID NO: 2;
a recombinant protein wherein a JO-13 MTD having an amino acid sequence represented by SEQ ID NO: 16 is fused to the N-terminus of a full-length RUNX3 having an amino acid sequence represented by SEQ ID NO: 2;
a recombinant protein wherein a JO-13 MTD having an amino acid sequence represented by SEQ ID NO: 16 is fused to the C-terminus of a full-length RUNX3 having an amino acid sequence represented by SEQ ID NO: 2;
a recombinant protein wherein a JO-13 MTD having an amino acid sequence represented by SEQ ID NO: 16 is fused to both termini of a full-length RUNX3 having an amino acid sequence represented by SEQ ID NO: 2;
a recombinant protein wherein a JO-108 MTD having an amino acid sequence represented by SEQ ID NO: 111 is fused to the N-terminus of a full-length RUNX3 having an amino acid sequence represented by SEQ ID NO: 2;
a recombinant protein wherein a JO-108 MTD having an amino acid sequence represented by SEQ ID NO: 111 is fused to the C-terminus of a full-length RUNX3 having an amino acid sequence represented by SEQ ID NO: 2; and
a recombinant protein wherein a JO-108 MTD having an amino acid sequence represented by SEQ ID NO: 111 is fused to both termini of a full-length RUNX3 having an amino acid sequence represented by SEQ ID NO: 2;
wherein a His-tag and a NLS derived from SV40 large T antigen are covalently coupled to the N-terminus of the above constructs.

7. The cell permeable RUNX3 recombinant protein according to claim 1, wherein the recombinant protein has an amino acid sequence selected from the group consisting of SEQ ID NOS: 199, 201, 203, 205, 207, 209, 211, 213, 215, 217, 219, 221, 223, 225, 227, 229, 231, 233, 235, 237 and 239.

8. A polynucleotide encoding the cell permeable RUNX3 recombinant protein according to claim 1.

9. The polynucleotide according to claim 8, wherein the polynucleotide has a nucleotide sequence selected from the group consisting of SEQ ID NOS: 198, 200, 202, 204, 206, 208, 210, 212, 214, 216, 218, 220, 222, 226, 228, 230, 232, 234, 236 and 238.

10. An expression vector comprising the polynucleotide according to claim 8.

11. The expression vector according to claim 10, wherein the expression vector is selected from the group consisting of:
pHM₁R3 which comprises a polynucleotide having a nucleotide sequence represented by SEQ ID NO: 198 which encodes cell permeable RUNX3 recombinant protein fused to a kFGF4-derived MTD;
pHR3M₁ which comprises a polynucleotide having a nucleotide sequence represented by SEQ ID NO: 200 which encodes a cell permeable RUNX3 recombinant protein fused to a kFGF4-derived MTD;
pHM₁R3M₁ which comprises a polynucleotide having a nucleotide sequence represented by SEQ ID NO: 202 which encodes a cell permeable RUNX3 recombinant protein fused to a kFGF4-derived MTD;
pHR3NM₁ which comprises a polynucleotide having a nucleotide sequence represented by SEQ ID NO: 204 which encodes a cell permeable RUNX3 recombinant protein fused to a kFGF4-derived MTD;
pHR3RM₁ which comprises a polynucleotide having a nucleotide sequence represented by SEQ ID NO: 206 which encodes a cell permeable RUNX3 recombinant protein fused to a kFGF4-derived MTD;
pHR3PM₁ which comprises a polynucleotide having a nucleotide sequence represented by SEQ ID NO: 208 which encodes a cell permeable RUNX3 recombinant protein fused to a kFGF4-derived MTD;
pHR3NRM₁ which comprises a polynucleotide having a nucleotide sequence represented by SEQ ID NO: 210 which encodes a cell permeable RUNX3 recombinant protein fused to a kFGF4-derived MTD;
pHR3RPM₁ which comprises a polynucleotide having a nucleotide sequence represented by SEQ ID NO: 212 which encodes a cell permeable RUNX3 recombinant protein fused to a kFGF4-derived MTD;
pHR3CRM₁ which comprises a polynucleotide having a nucleotide sequence represented by SEQ ID NO: 214 which encodes a cell permeable RUNX3 recombinant protein fused to a kFGF4-derived MTD;
pHM₂R3 which comprises a polynucleotide having a nucleotide sequence represented by SEQ ID NO: 216 which encodes a cell permeable RUNX3 recombinant protein fused to a JO-57 MTD;
pHR3M₂ which comprises a polynucleotide having a nucleotide sequence represented by SEQ ID NO: 218 which encodes a cell permeable RUNX3 recombinant protein fused to a JO-57 MTD;
pHM₂R3M₂ which comprises a polynucleotide having a nucleotide sequence represented by SEQ ID NO: 220 which encodes a cell permeable RUNX3 recombinant protein fused to a JO-57 MTD;
pHM₃R3 which comprises a polynucleotide having a nucleotide sequence represented by SEQ ID NO: 222 which encodes a cell permeable RUNX3 recombinant protein fused to a JO-57 MTD;
pHR3M₃ which comprises a polynucleotide having a nucleotide sequence represented by SEQ ID NO: 224 which encodes a cell permeable RUNX3 recombinant protein fused to a JO-85 MTD;
pHM₃R3M₃ which comprises a polynucleotide having a nucleotide sequence represented by SEQ ID NO: 226 which encodes a cell permeable RUNX3 recombinant protein fused to a JO-85 MTD;
pHM₄R3 which comprises a polynucleotide having a nucleotide sequence represented by SEQ ID NO: 228 which encodes a cell permeable RUNX3 recombinant protein fused to a JO-13 MTD;
pHR3M₄ which comprises a polynucleotide having a nucleotide sequence represented by SEQ ID NO: 230 which encodes a cell permeable RUNX3 recombinant protein fused to a JO-13 MTD;
pHM₄R3M₄ which comprises a polynucleotide having a nucleotide sequence represented by SEQ ID NO: 232 which encodes a cell permeable RUNX3 recombinant protein fused to a JO-13 MTD;
pHM₅R3 which comprises a polynucleotide having a nucleotide sequence represented by SEQ ID NO: 234 which encodes a cell permeable RUNX3 recombinant protein fused to a JO-108 MTD;
pHR3M₅ which comprises a polynucleotide having a nucleotide sequence represented by SEQ ID NO: 236 which encodes a cell permeable RUNX3 recombinant protein fused to a JO-108 MTD; and
pHM₅R3M₅ which comprises a polynucleotide having a nucleotide sequence represented by SEQ ID NO: 238 which encodes a cell permeable RUNX3 recombinant protein fused to a JO-108 MTD;

12. A transformant comprising the expression vector according to claim 10.

13. The transformant according to claim 12, wherein the transformant is E. coli DH5α/HM₂R3 (KCTC-11408BP).

14. The transformant according to claim 12, wherein the transformant is E. coli DH5α/HM₃R3 (KCTC-11409BP).

15. A method of producing a cell permeable RUNX3 recombinant protein comprising the following steps of:
1) culturing the transformant according to claim 12 to express a cell permeable RUNX3 recombinant protein; and
2) purifying the expressed cell permeable RUNX3 recombinant protein from the culture containing the transformant.

16. A pharmaceutical composition for treating RUNX3 deficiency or failure comprising the cell permeable RUNX3 recombinant protein according to claim 1 as an effective ingredient and a pharmaceutically acceptable carrier.

17. The pharmaceutical composition according to claim 16, which is used for preventing or treating tumor growth and metastasis, wherein the tumor is selected from the group consisting of gastric cancer, pancreatic cancer, lung cancer, colon cancer and liver cancer.


SEQUENCE LISTING (261 sequences)

SEQ ID NO: 1 - DNA, 1248 bp, Homo sapiens
  RINX3 cDNA gene
atgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactga

SEQ ID NO: 2 - protein, 415 aa, Homo sapiens
  RUNX3 protein
MRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 3 - protein, 12 aa, Artificial Sequence
  Description of Artificial Sequence Synthetic kFGF-4 derived MTD peptide
AAVLLPVLLAAP

SEQ ID NO: 4 - protein, 12 aa, Artificial Sequence
  Description of Artificial Sequence Synthetic JO-01 MTD peptide
AVVVCAIVLAAP

SEQ ID NO: 5 - protein, 12 aa, Artificial Sequence
  Description of Artificial Sequence Synthetic JO-02 MTD peptide
PLALLVLLLLGP

SEQ ID NO: 6 - protein, 11 aa, Artificial Sequence
  Description of Artificial Sequence Synthetic JO-03 MTD peptide
LLLAFALLCLP

SEQ ID NO: 7 - protein, 13 aa, Artificial Sequence
  Description of Artificial Sequence Synthetic JO-04 MTD peptide
LLGALAAVLLALA

SEQ ID NO: 8 - protein, 17 aa, Artificial Sequence
  Description of Artificial Sequence Synthetic JO-05 MTD peptide
PVLLALGVGLVLLGLAV

SEQ ID NO: 9 - protein, 9 aa, Artificial Sequence
  Description of Artificial Sequence Synthetic JO-06 MTD peptide
AAAAVLLAA

SEQ ID NO: 10 - protein, 8 aa, Artificial Sequence
  Description of Artificial Sequence Synthetic JO-07 MTD peptide
IVVAVVVI

SEQ ID NO: 11 - protein, 9 aa, Artificial Sequence
  Description of Artificial Sequence Synthetic JO-08 MTD peptide
AVLAPVVAV

SEQ ID NO: 12 - protein, 13 aa, Artificial Sequence
  Description of Artificial Sequence Synthetic JO-09 MTD peptide
LAVCGLPVVALLA

SEQ ID NO: 13 - protein, 16 aa, Artificial Sequence
  Description of Artificial Sequence Synthetic JO-10 MTD peptide
LGGAVVAAPVAAAVAP

SEQ ID NO: 14 - protein, 13 aa, Artificial Sequence
  Description of Artificial Sequence Synthetic JO-11 MTD peptide
LLLVLAVLLAVLP

SEQ ID NO: 15 - protein, 12 aa, Artificial Sequence
  Description of Artificial Sequence Synthetic JO-12 MTD peptide
LLILLLLPLLIV

SEQ ID NO: 16 - protein, 11 aa, Artificial Sequence
  Description of Artificial Sequence Synthetic JO-13 MTD peptide
LAAAALAVLPL

SEQ ID NO: 17 - protein, 14 aa, Artificial Sequence
  Description of Artificial Sequence Synthetic JO-14 MTD peptide
FLMLLLPLLLLLVA

SEQ ID NO: 18 - protein, 15 aa, Artificial Sequence
  Description of Artificial Sequence Synthetic JO-15 MTD peptide
AAAAAALGLAAAVPA

SEQ ID NO: 19 - protein, 14 aa, Artificial Sequence
  Description of Artificial Sequence Synthetic JO-16 MTD peptide
LLLAALLLIAFAAV

SEQ ID NO: 20 - protein, 14 aa, Artificial Sequence
  Description of Artificial Sequence Synthetic JO-17 MTD peptide
ALAAVVLIPLGIAA

SEQ ID NO: 21 - protein, 16 aa, Artificial Sequence
  Description of Artificial Sequence Synthetic JO-18 MTD peptide
AALALGVAAAPAAAPA

SEQ ID NO: 22 - protein, 14 aa, Artificial Sequence
  Description of Artificial Sequence Synthetic JO-19 MTD peptide
AALIGAVLAPVVAV

SEQ ID NO: 23 - protein, 15 aa, Artificial Sequence
  Description of Artificial Sequence Synthetic JO-20 MTD peptide
AAGIAVAIAAIVPLA

SEQ ID NO: 24 - protein, 12 aa, Artificial Sequence
  Description of Artificial Sequence Synthetic JO-21 MTD peptide
IAVAIAAIVPLA

SEQ ID NO: 25 - protein, 15 aa, Artificial Sequence
  Description of Artificial Sequence Synthetic JO-22 MTD peptide
VAMAAAAVLAAPALA

SEQ ID NO: 26 - protein, 11 aa, Artificial Sequence
  Description of Artificial Sequence Synthetic JO-23 MTD peptide
LAVLVLLVLLP

SEQ ID NO: 27 - protein, 9 aa, Artificial Sequence
  Description of Artificial Sequence Synthetic JO-24 MTD peptide
VVAVLAPVL

SEQ ID NO: 28 - protein, 12 aa, Artificial Sequence
  Description of Artificial Sequence Synthetic JO-25 MTD peptide
AALLLPLLLLLP

SEQ ID NO: 29 - protein, 10 aa, Artificial Sequence
  Description of Artificial Sequence Synthetic JO-26 MTD peptide
PAAVAALLVI

SEQ ID NO: 30 - protein, 8 aa, Artificial Sequence
  Description of Artificial Sequence Synthetic JO-27 MTD peptide
LLIAALLP

SEQ ID NO: 31 - protein, 12 aa, Artificial Sequence
  Description of Artificial Sequence Synthetic JO-28 MTD peptide
AAVVLLPLAAAP

SEQ ID NO: 32 - protein, 10 aa, Artificial Sequence
  Description of Artificial Sequence Synthetic JO-29 MTD peptide
AAAAAALLVP

SEQ ID NO: 33 - protein, 8 aa, Artificial Sequence
  Description of Artificial Sequence Synthetic JO-30 MTD peptide
LPVVALLA

SEQ ID NO: 34 - protein, 11 aa, Artificial Sequence
  Description of Artificial Sequence Synthetic JO-31 MTD peptide
AAALAAPLALP

SEQ ID NO: 35 - protein, 9 aa, Artificial Sequence
  Description of Artificial Sequence Synthetic JO-32 MTD peptide
LLLALLLAA

SEQ ID NO: 36 - protein, 8 aa, Artificial Sequence
  Description of Artificial Sequence Synthetic JO-33 MTD peptide
AVAVVALL

SEQ ID NO: 37 - protein, 11 aa, Artificial Sequence
  Description of Artificial Sequence Synthetic JO-34 MTD peptide
LLLIIVLLIVP

SEQ ID NO: 38 - protein, 9 aa, Artificial Sequence
  Description of Artificial Sequence Synthetic JO-35 MTD peptide
LALAAAVVP

SEQ ID NO: 39 - protein, 10 aa, Artificial Sequence
  Description of Artificial Sequence Synthetic JO-36 MTD peptide
PAALALLLVA

SEQ ID NO: 40 - protein, 17 aa, Artificial Sequence
  Description of Artificial Sequence Synthetic JO-37 MTD peptide
IVALLLVPLVLAIAAVL

SEQ ID NO: 41 - protein, 8 aa, Artificial Sequence
  Description of Artificial Sequence Synthetic JO-38 MTD peptide
IVALLLVP

SEQ ID NO: 42 - protein, 10 aa, Artificial Sequence
  Description of Artificial Sequence Synthetic JO-39 MTD peptide
PLVLAIAAVL

SEQ ID NO: 43 - protein, 9 aa, Artificial Sequence
  Description of Artificial Sequence Synthetic JO-40 MTD peptide
PLVLAALVA

SEQ ID NO: 44 - protein, 8 aa, Artificial Sequence
  Description of Artificial Sequence Synthetic JO-41 MTD peptide
AAALLAVA

SEQ ID NO: 45 - protein, 8 aa, Artificial Sequence
  Description of Artificial Sequence Synthetic JO-42 MTD peptide
PLLLLALA

SEQ ID NO: 46 - protein, 7 aa, Artificial Sequence
  Description of Artificial Sequence Synthetic JO-43 MTD peptide
ALALVVA

SEQ ID NO: 47 - protein, 8 aa, Artificial Sequence
  Description of Artificial Sequence Synthetic JO-44 MTD peptide
VAAVVVAA

SEQ ID NO: 48 - protein, 9 aa, Artificial Sequence
  Description of Artificial Sequence Synthetic JO-45 MTD peptide
PLLPLLLLV

SEQ ID NO: 49 - protein, 13 aa, Artificial Sequence
  Description of Artificial Sequence Synthetic JO-46 MTD peptide
VVLVVVLPLAVLA

SEQ ID NO: 50 - protein, 10 aa, Artificial Sequence
  Description of Artificial Sequence Synthetic JO-47 MTD peptide
AAAVPVLVAA

SEQ ID NO: 51 - protein, 12 aa, Artificial Sequence
  Description of Artificial Sequence Synthetic JO-48 MTD peptide
PALLLLLLAAVV

SEQ ID NO: 52 - protein, 12 aa, Artificial Sequence
  Description of Artificial Sequence Synthetic JO-49 MTD peptide
PLAILLLLLIAP

SEQ ID NO: 53 - protein, 13 aa, Artificial Sequence
  Description of Artificial Sequence Synthetic JO-50 MTD peptide
PLLALVLLLALIA

SEQ ID NO: 54 - protein, 12 aa, Artificial Sequence
  Description of Artificial Sequence Synthetic JO-51 MTD peptide
VVAVLALVLAAL

SEQ ID NO: 55 - protein, 9 aa, Artificial Sequence
  Description of Artificial Sequence Synthetic JO-52 MTD peptide
PLLLLLPAL

SEQ ID NO: 56 - protein, 12 aa, Artificial Sequence
  Description of Artificial Sequence Synthetic JO-53 MTD peptide
LAAVAALAVVVP

SEQ ID NO: 57 - protein, 12 aa, Artificial Sequence
  Description of Artificial Sequence Synthetic JO-54peptide
LLLLVLILPLAA

SEQ ID NO: 58 - protein, 9 aa, Artificial Sequence
  Description of Artificial Sequence Synthetic JO-55 MTD peptide
LAVVVVAAV

SEQ ID NO: 59 - protein, 9 aa, Artificial Sequence
  Description of Artificial Sequence Synthetic JO-56 MTD peptide
VLLAAALIA

SEQ ID NO: 60 - protein, 10 aa, Artificial Sequence
  Description of Artificial Sequence Synthetic JO-57 MTD peptide
LIALLAAPLA

SEQ ID NO: 61 - protein, 8 aa, Artificial Sequence
  Description of Artificial Sequence Synthetic JO-58 MTD peptide
LALLLLAA

SEQ ID NO: 62 - protein, 12 aa, Artificial Sequence
  Description of Artificial Sequence Synthetic JO-59 MTD peptide
LLAAALLLLLLA

SEQ ID NO: 63 - protein, 10 aa, Artificial Sequence
  Description of Artificial Sequence Synthetic JO-60 MTD peptide
VIIALIVIVA

SEQ ID NO: 64 - protein, 11 aa, Artificial Sequence
  Description of Artificial Sequence Synthetic JO-61 MTD peptide
VVLVVAAVLAL

SEQ ID NO: 65 - protein, 9 aa, Artificial Sequence
  Description of Artificial Sequence Synthetic JO-62 MTD peptide
VAVAIAVVL

SEQ ID NO: 66 - protein, 13 aa, Artificial Sequence
  Description of Artificial Sequence Synthetic JO-63 MTD peptide
PLIVVVAAAVVAV

SEQ ID NO: 67 - protein, 11 aa, Artificial Sequence
  Description of Artificial Sequence Synthetic JO-64 MTD peptide
PLAVAVAAVAA

SEQ ID NO: 68 - protein, 10 aa, Artificial Sequence
  Description of Artificial Sequence Synthetic JO-65 MTD peptide
AAIALVAVVL

SEQ ID NO: 69 - protein, 10 aa, Artificial Sequence
  Description of Artificial Sequence Synthetic JO-66 MTD peptide
AAALAAIAVI

SEQ ID NO: 70 - protein, 8 aa, Artificial Sequence
  Description of Artificial Sequence Synthetic JO-67 MTD peptide
AAAPAVAA

SEQ ID NO: 71 - protein, 7 aa, Artificial Sequence
  Description of Artificial Sequence Synthetic JO-68 MTD peptide
LLLAALP

SEQ ID NO: 72 - protein, 8 aa, Artificial Sequence
  Description of Artificial Sequence Synthetic JO-69 MTD peptide
ALLAVVAA

SEQ ID NO: 73 - protein, 10 aa, Artificial Sequence
  Description of Artificial Sequence Synthetic JO-70 MTD peptide
AVVVVLPILL

SEQ ID NO: 74 - protein, 9 aa, Artificial Sequence
  Description of Artificial Sequence Synthetic JO-71 MTD peptide
ALALLLLVP

SEQ ID NO: 75 - protein, 11 aa, Artificial Sequence
  Description of Artificial Sequence Synthetic JO-72 MTD peptide
LVVLLAALLVL

SEQ ID NO: 76 - protein, 8 aa, Artificial Sequence
  Description of Artificial Sequence Synthetic JO-73 MTD peptide
PVLLLLAP

SEQ ID NO: 77 - protein, 8 aa, Artificial Sequence
  Description of Artificial Sequence Synthetic JO-74 MTD peptide
ALAVVAAP

SEQ ID NO: 78 - protein, 8 aa, Artificial Sequence
  Description of Artificial Sequence Synthetic JO-75 MTD peptide
VIVALLAV

SEQ ID NO: 79 - protein, 8 aa, Artificial Sequence
  Description of Artificial Sequence Synthetic JO-76 MTD peptide
ALVLPLAP

SEQ ID NO: 80 - protein, 9 aa, Artificial Sequence
  Description of Artificial Sequence Synthetic JO-77 MTD peptide
AVALLILAV

SEQ ID NO: 81 - protein, 7 aa, Artificial Sequence
  Description of Artificial Sequence Synthetic JO-78 MTD peptide
VLLAVIP

SEQ ID NO: 82 - protein, 13 aa, Artificial Sequence
  Description of Artificial Sequence Synthetic JO-79 MTD peptide
LIVAAVVVVAVLI

SEQ ID NO: 83 - protein, 7 aa, Artificial Sequence
  Description of Artificial Sequence Synthetic JO-80 MTD peptide
AVVVAAP

SEQ ID NO: 84 - protein, 9 aa, Artificial Sequence
  Description of Artificial Sequence Synthetic JO-81 MTD peptide
LAAVLLLIP

SEQ ID NO: 85 - protein, 10 aa, Artificial Sequence
  Description of Artificial Sequence Synthetic JO-82 MTD peptide
LLLLLLAVVP

SEQ ID NO: 86 - protein, 11 aa, Artificial Sequence
  Description of Artificial Sequence Synthetic JO-83 MTD peptide
AVALVAVVAVA

SEQ ID NO: 87 - protein, 9 aa, Artificial Sequence
  Description of Artificial Sequence Synthetic JO-84 MTD peptide
LVAALLAVL

SEQ ID NO: 88 - protein, 11 aa, Artificial Sequence
  Description of Artificial Sequence Synthetic JO-85 MTD peptide
LLAAAAALLLA

SEQ ID NO: 89 - protein, 8 aa, Artificial Sequence
  Description of Artificial Sequence Synthetic JO-86 MTD peptide
LAVLAAAP

SEQ ID NO: 90 - protein, 13 aa, Artificial Sequence
  Description of Artificial Sequence Synthetic JO-87 MTD peptide
VVVLLVLLALVVV

SEQ ID NO: 91 - protein, 7 aa, Artificial Sequence
  Description of Artificial Sequence Synthetic JO-88 MTD peptide
VVIAVVP

SEQ ID NO: 92 - protein, 10 aa, Artificial Sequence
  Description of Artificial Sequence Synthetic JO-89 MTD peptide
LAAVAALAVV

SEQ ID NO: 93 - protein, 9 aa, Artificial Sequence
  Description of Artificial Sequence Synthetic JO-90 MTD peptide
VLLVLLALV

SEQ ID NO: 94 - protein, 8 aa, Artificial Sequence
  Description of Artificial Sequence Synthetic JO-91 MTD peptide
PVLVPAVP

SEQ ID NO: 95 - protein, 8 aa, Artificial Sequence
  Description of Artificial Sequence Synthetic JO-92 MTD peptide
PALALALA

SEQ ID NO: 96 - protein, 8 aa, Artificial Sequence
  Description of Artificial Sequence Synthetic JO-93 MTD peptide
AAAAPALA

SEQ ID NO: 97 - protein, 9 aa, Artificial Sequence
  Description of Artificial Sequence Synthetic JO-94 MTD peptide
IVLPVLAAP

SEQ ID NO: 98 - protein, 10 aa, Artificial Sequence
  Description of Artificial Sequence Synthetic JO-95 MTD peptide
LVLLLLPLLI

SEQ ID NO: 99 - protein, 11 aa, Artificial Sequence
  Description of Artificial Sequence Synthetic JO-96 MTD peptide
LAAVAPALAVV

SEQ ID NO: 100 - protein, 8 aa, Artificial Sequence
  Description of Artificial Sequence Synthetic JO-97 MTD peptide
ILVLVLPI

SEQ ID NO: 101 - protein, 9 aa, Artificial Sequence
  Description of Artificial Sequence Synthetic JO-98 MTD peptide
ILLPLLLLP

SEQ ID NO: 102 - protein, 10 aa, Artificial Sequence
  Description of Artificial Sequence Synthetic JO-99 MTD peptide
IAPAVVAALP

SEQ ID NO: 103 - protein, 12 aa, Artificial Sequence
  Description of Artificial Sequence Synthetic JO-100 MTD peptide
LLLVAVVPLLVP

SEQ ID NO: 104 - protein, 9 aa, Artificial Sequence
  Description of Artificial Sequence Synthetic JO-101 MTD peptide
LILLLLPII

SEQ ID NO: 105 - protein, 10 aa, Artificial Sequence
  Description of Artificial Sequence Synthetic JO-102 MTD peptide
AVLAAPAVLV

SEQ ID NO: 106 - protein, 9 aa, Artificial Sequence
  Description of Artificial Sequence Synthetic JO-103 MTD peptide
LALPVLLLA

SEQ ID NO: 107 - protein, 7 aa, Artificial Sequence
  Description of Artificial Sequence Synthetic JO-104 MTD peptide
LALALLL

SEQ ID NO: 108 - protein, 9 aa, Artificial Sequence
  Description of Artificial Sequence Synthetic JO-105 MTD peptide
VAVPLLVVA

SEQ ID NO: 109 - protein, 11 aa, Artificial Sequence
  Description of Artificial Sequence Synthetic JO-106 MTD peptide
AVAVAPVAAAA

SEQ ID NO: 110 - protein, 11 aa, Artificial Sequence
  Description of Artificial Sequence Synthetic JO-107 MTD peptide
AAAVVAAVPAA

SEQ ID NO: 111 - protein, 9 aa, Artificial Sequence
  Description of Artificial Sequence Synthetic JO-108 MTD peptide
ALLAALLAP

SEQ ID NO: 112 - protein, 7 aa, Artificial Sequence
  Description of Artificial Sequence Synthetic JO-109 MTD peptide
LLALLVP

SEQ ID NO: 113 - protein, 14 aa, Artificial Sequence
  Description of Artificial Sequence Synthetic JO-110 MTD peptide
ALLAALLALLALLV

SEQ ID NO: 114 - protein, 11 aa, Artificial Sequence
  Description of Artificial Sequence Synthetic JO-111 MTD peptide
AAALPLLVLLP

SEQ ID NO: 115 - protein, 10 aa, Artificial Sequence
  Description of Artificial Sequence Synthetic JO-112 MTD peptide
AAAVPAALAP

SEQ ID NO: 116 - protein, 10 aa, Artificial Sequence
  Description of Artificial Sequence Synthetic JO-113 MTD peptide
AALAVAALAA

SEQ ID NO: 117 - protein, 8 aa, Artificial Sequence
  Description of Artificial Sequence Synthetic JO-114 MTD peptide
AVLAAAVP

SEQ ID NO: 118 - protein, 8 aa, Artificial Sequence
  Description of Artificial Sequence Synthetic JO-115 MTD peptide
VAALPAPA

SEQ ID NO: 119 - protein, 11 aa, Artificial Sequence
  Description of Artificial Sequence Synthetic JO-116 MTD peptide
ALALAVPAVLP

SEQ ID NO: 120 - protein, 11 aa, Artificial Sequence
  Description of Artificial Sequence Synthetic JO-117 MTD peptide
AALLPAAVAVP

SEQ ID NO: 121 - protein, 8 aa, Artificial Sequence
  Description of Artificial Sequence Synthetic JO-118 MTD peptide
AVVVALAP

SEQ ID NO: 122 - protein, 14 aa, Artificial Sequence
  Description of Artificial Sequence Synthetic JO-119 MTD peptide
AAAVALPAAAALLA

SEQ ID NO: 123 - protein, 13 aa, Artificial Sequence
  Description of Artificial Sequence Synthetic JO-120 MTD peptide
AVVLPLALVAVAP

SEQ ID NO: 124 - protein, 8 aa, Artificial Sequence
  Description of Artificial Sequence Synthetic JO-121 MTD peptide
LVALPLLP

SEQ ID NO: 125 - protein, 10 aa, Artificial Sequence
  Description of Artificial Sequence Synthetic JO-122 MTD peptide
VVVPLLLIVP

SEQ ID NO: 126 - protein, 8 aa, Artificial Sequence
  Description of Artificial Sequence Synthetic JO-123 MTD peptide
LAVVLAVP

SEQ ID NO: 127 - protein, 10 aa, Artificial Sequence
  Description of Artificial Sequence Synthetic JO-124 MTD peptide
LLAVPILLVP

SEQ ID NO: 128 - protein, 8 aa, Artificial Sequence
  Description of Artificial Sequence Synthetic JO-125 MTD peptide
LVALVLLP

SEQ ID NO: 129 - protein, 14 aa, Artificial Sequence
  Description of Artificial Sequence Synthetic JO-126 MTD peptide
LVLLLAVLLLAVLP

SEQ ID NO: 130 - protein, 12 aa, Artificial Sequence
  Description of Artificial Sequence Synthetic JO-127 MTD peptide
LLAPVVALVILP

SEQ ID NO: 131 - protein, 13 aa, Artificial Sequence
  Description of Artificial Sequence Synthetic JO-128 MTD peptide
VLAVLAVPVLLLP

SEQ ID NO: 132 - protein, 10 aa, Artificial Sequence
  Description of Artificial Sequence Synthetic JO-129 MTD peptide
VVIAVVPVVV

SEQ ID NO: 133 - protein, 11 aa, Artificial Sequence
  Description of Artificial Sequence Synthetic JO-130 MTD peptide
LLVLLALVVVP

SEQ ID NO: 134 - protein, 11 aa, Artificial Sequence
  Description of Artificial Sequence Synthetic JO-131 MTD peptide
VLLALPVVAAP

SEQ ID NO: 135 - protein, 12 aa, Artificial Sequence
  Description of Artificial Sequence Synthetic JO-132 MTD peptide
AVVVPAIVLAAP

SEQ ID NO: 136 - protein, 11 aa, Artificial Sequence
  Description of Artificial Sequence Synthetic JO-133 MTD peptide
AVLVPAAALVP

SEQ ID NO: 137 - protein, 10 aa, Artificial Sequence
  Description of Artificial Sequence Synthetic JO-134 MTD peptide
VVAALPLVLP

SEQ ID NO: 138 - protein, 10 aa, Artificial Sequence
  Description of Artificial Sequence Synthetic JO-135 MTD peptide
AAVALPAAAP

SEQ ID NO: 139 - protein, 8 aa, Artificial Sequence
  Description of Artificial Sequence Synthetic JO-136 MTD peptide
LIALPLLP

SEQ ID NO: 140 - protein, 13 aa, Artificial Sequence
  Description of Artificial Sequence Synthetic JO-137 MTD peptide
LLALPLVLVLALP

SEQ ID NO: 141 - protein, 9 aa, Artificial Sequence
  Description of Artificial Sequence Synthetic JO-138 MTD peptide
IVPLLLAAP

SEQ ID NO: 142 - protein, 10 aa, Artificial Sequence
  Description of Artificial Sequence Synthetic JO-139 MTD peptide
LLLAPLLLAP

SEQ ID NO: 143 - protein, 10 aa, Artificial Sequence
  Description of Artificial Sequence Synthetic JO-140 MTD peptide
LAALPVAAVP

SEQ ID NO: 144 - protein, 10 aa, Artificial Sequence
  Description of Artificial Sequence Synthetic JO-141 MTD peptide
ALAVIVLVLL

SEQ ID NO: 145 - protein, 10 aa, Artificial Sequence
  Description of Artificial Sequence Synthetic JO-142 MTD peptide
LALLLPAALI

SEQ ID NO: 146 - protein, 11 aa, Artificial Sequence
  Description of Artificial Sequence Synthetic JO-143 MTD peptide
ALLPLLAVVLP

SEQ ID NO: 147 - protein, 10 aa, Artificial Sequence
  Description of Artificial Sequence Synthetic JO-144 MTD peptide
AIAVPVLAAP

SEQ ID NO: 148 - protein, 10 aa, Artificial Sequence
  Description of Artificial Sequence Synthetic JO-145 MTD peptide
AAAPVLLLLL

SEQ ID NO: 149 - protein, 11 aa, Artificial Sequence
  Description of Artificial Sequence Synthetic JO-146 MTD peptide
AAAVAVLALAP

SEQ ID NO: 150 - protein, 11 aa, Artificial Sequence
  Description of Artificial Sequence Synthetic JO-147 MTD peptide
AALAALVVAAP

SEQ ID NO: 151 - protein, 12 aa, Artificial Sequence
  Description of Artificial Sequence Synthetic JO-148 MTD peptide
AALAAVPLALAP

SEQ ID NO: 152 - protein, 13 aa, Artificial Sequence
  Description of Artificial Sequence Synthetic JO-149 MTD peptide
ALAVAAPALALLP

SEQ ID NO: 153 - protein, 8 aa, Artificial Sequence
  Description of Artificial Sequence Synthetic JO-150 MTD peptide
AALPAAAP

SEQ ID NO: 154 - protein, 9 aa, Artificial Sequence
  Description of Artificial Sequence Synthetic JO-151 MTD peptide
AAAPVAAVP

SEQ ID NO: 155 - protein, 10 aa, Artificial Sequence
  Description of Artificial Sequence Synthetic JO-152 MTD peptide
LLAVLLALLP

SEQ ID NO: 156 - protein, 10 aa, Artificial Sequence
  Description of Artificial Sequence Synthetic JO-153 MTD peptide
VLALLVAVVP

SEQ ID NO: 157 - protein, 9 aa, Artificial Sequence
  Description of Artificial Sequence Synthetic JO-154 MTD peptide
ALVVPAAVP

SEQ ID NO: 158 - protein, 9 aa, Artificial Sequence
  Description of Artificial Sequence Synthetic JO-155 MTD peptide
AVVLPLLLP

SEQ ID NO: 159 - protein, 10 aa, Artificial Sequence
  Description of Artificial Sequence Synthetic JO-156 MTD peptide
AVIPVAVLVP

SEQ ID NO: 160 - protein, 11 aa, Artificial Sequence
  Description of Artificial Sequence Synthetic JO-157 MTD peptide
AAAVPAAVLAP

SEQ ID NO: 161 - protein, 11 aa, Artificial Sequence
  Description of Artificial Sequence Synthetic JO-158 MTD peptide
VAVPVVLAILP

SEQ ID NO: 162 - protein, 12 aa, Artificial Sequence
  Description of Artificial Sequence Synthetic JO-159 MTD peptide
IAIAAIPAILAL

SEQ ID NO: 163 - protein, 10 aa, Artificial Sequence
  Description of Artificial Sequence Synthetic JO-160 MTD peptide
ALIAPALAAP

SEQ ID NO: 164 - protein, 10 aa, Artificial Sequence
  Description of Artificial Sequence Synthetic JO-161 MTD peptide
AAIALVAPAL

SEQ ID NO: 165 - protein, 9 aa, Artificial Sequence
  Description of Artificial Sequence Synthetic JO-162 MTD peptide
LAPAVAAAP

SEQ ID NO: 166 - protein, 15 aa, Artificial Sequence
  Description of Artificial Sequence Synthetic JO-163 MTD peptide
VAIIVPAVVAIALII

SEQ ID NO: 167 - protein, 9 aa, Artificial Sequence
  Description of Artificial Sequence Synthetic JO-164 MTD peptide
AVVAIALII

SEQ ID NO: 168 - protein, 9 aa, Artificial Sequence
  Description of Artificial Sequence Synthetic JO-165 MTD peptide
LAAVPAAAP

SEQ ID NO: 169 - protein, 10 aa, Artificial Sequence
  Description of Artificial Sequence Synthetic JO-166 MTD peptide
AVAALPLAAP

SEQ ID NO: 170 - protein, 9 aa, Artificial Sequence
  Description of Artificial Sequence Synthetic JO-167 MTD peptide
LAAPAAAAP

SEQ ID NO: 171 - protein, 12 aa, Artificial Sequence
  Description of Artificial Sequence Synthetic JO-168 MTD peptide
LAAVVPVAAAVP

SEQ ID NO: 172 - protein, 9 aa, Artificial Sequence
  Description of Artificial Sequence Synthetic JO-169 MTD peptide
VAAPAAAAP

SEQ ID NO: 173 - protein, 8 aa, Artificial Sequence
  Description of Artificial Sequence Synthetic JO-170 MTD peptide
AVPVPVPL

SEQ ID NO: 174 - protein, 10 aa, Artificial Sequence
  Description of Artificial Sequence Synthetic JO-171 MTD peptide
LLILPIVLLP

SEQ ID NO: 175 - protein, 11 aa, Artificial Sequence
  Description of Artificial Sequence Synthetic JO-172 MTD peptide
ALALPALAIAP

SEQ ID NO: 176 - protein, 9 aa, Artificial Sequence
  Description of Artificial Sequence Synthetic JO-173 MTD peptide
AVIPILAVP

SEQ ID NO: 177 - protein, 11 aa, Artificial Sequence
  Description of Artificial Sequence Synthetic JO-174 MTD peptide
LILLLPAVALP

SEQ ID NO: 178 - protein, 10 aa, Artificial Sequence
  Description of Artificial Sequence Synthetic JO-175 MTD peptide
IVLAPVPAAA

SEQ ID NO: 179 - protein, 11 aa, Artificial Sequence
  Description of Artificial Sequence Synthetic JO-176 MTD peptide
VVVVPVLAAAA

SEQ ID NO: 180 - protein, 7 aa, Artificial Sequence
  Description of Artificial Sequence Synthetic JO-177 MTD peptide
LVAVAAP

SEQ ID NO: 181 - protein, 10 aa, Artificial Sequence
  Description of Artificial Sequence Synthetic JO-178 MTD peptide
LVLAAPAALP

SEQ ID NO: 182 - protein, 9 aa, Artificial Sequence
  Description of Artificial Sequence Synthetic JO-179 MTD peptide
LIAPAAAVP

SEQ ID NO: 183 - protein, 10 aa, Artificial Sequence
  Description of Artificial Sequence Synthetic JO-180 MTD peptide
ALAALPIALP

SEQ ID NO: 184 - protein, 9 aa, Artificial Sequence
  Description of Artificial Sequence Synthetic JO-181 MTD peptide
AVLLLPAAA

SEQ ID NO: 185 - protein, 9 aa, Artificial Sequence
  Description of Artificial Sequence Synthetic JO-182 MTD peptide
IALALLPLL

SEQ ID NO: 186 - protein, 10 aa, Artificial Sequence
  Description of Artificial Sequence Synthetic JO-183 MTD peptide
VLLAAALIAP

SEQ ID NO: 187 - protein, 11 aa, Artificial Sequence
  Description of Artificial Sequence Synthetic JO-184 MTD peptide
APAVLPPVVVI

SEQ ID NO: 188 - protein, 9 aa, Artificial Sequence
  Description of Artificial Sequence Synthetic JO-185 MTD peptide
VVGLLVAAL

SEQ ID NO: 189 - protein, 11 aa, Artificial Sequence
  Description of Artificial Sequence Synthetic JO-186 MTD peptide
AAIAAAAPLAA

SEQ ID NO: 190 - protein, 7 aa, Artificial Sequence
  Description of Artificial Sequence Synthetic JO-187 MTD peptide
LLLAVAP

SEQ ID NO: 191 - protein, 10 aa, Artificial Sequence
  Description of Artificial Sequence Synthetic JO-188 MTD peptide
LILLLPLAAL

SEQ ID NO: 192 - protein, 8 aa, Artificial Sequence
  Description of Artificial Sequence Synthetic JO-189 MTD peptide
ALLLLVLA

SEQ ID NO: 193 - protein, 9 aa, Artificial Sequence
  Description of Artificial Sequence Synthetic JO-190 MTD peptide
LLLLLLPLA

SEQ ID NO: 194 - protein, 8 aa, Artificial Sequence
  Description of Artificial Sequence Synthetic JO-191 MTD peptide
LALPLLLP

SEQ ID NO: 195 - protein, 8 aa, Artificial Sequence
  Description of Artificial Sequence Synthetic JO-192 MTD peptide
LLVLPLLI

SEQ ID NO: 196 - protein, 9 aa, Artificial Sequence
  Description of Artificial Sequence Synthetic JO-193 MTD peptide
LPLLPAALV

SEQ ID NO: 197 - protein, 5 aa, Artificial Sequence
  Description of Artificial Sequence Synthetic SV40 large T antigen-derived NLS peptide
KKKRK

SEQ ID NO: 198 - DNA, 1359 bp, Artificial Sequence
  Description of Artificial Sequence Synthetic HM1R3 polynucleotide
atgggcagcagccatcatcatcatcatcacagcagcggcctggtgccgcgcggcagccatatgaagaagaagaggaaggcagccgttcttctccctgttcttcttgccgcaccccgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactga

SEQ ID NO: 199 - protein, 452 aa, Artificial Sequence
  Description of Artificial Sequence Synthetic HM1R3 recombinant polypeptide
MGSSHHHHHHSSGLVPRGSHMKKKRKAAVLLPVLLAAPRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 200 - DNA, 1359 bp, Artificial Sequence
  Description of Artificial Sequence Synthetic HR3M1 polynucleotide
atgggcagcagccatcatcatcatcatcacagcagcggcctggtgccgcgcggcagccatatgaagaagaagaggaagcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacgcagccgttcttctccctgttcttcttgccgcaccctga

SEQ ID NO: 201 - protein, 452 aa, Artificial Sequence
  Description of Artificial Sequence Synthetic HR3M1 recombinant polypeptide
MGSSHHHHHHSSGLVPRGSHMKKKRKRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYAAVLLPVLLAAP

SEQ ID NO: 202 - DNA, 1395 bp, Artificial Sequence
  Description of Artificial Sequence Synthetic HM1R3M1 polynucleotide
atgggcagcagccatcatcatcatcatcacagcagcggcctggtgccgcgcggcagccatatgaagaagaagaggaaggcagccgttcttctccctgttcttcttgccgcaccccgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacgcagccgttcttctccctgttcttcttgccgcaccctga

SEQ ID NO: 203 - protein, 464 aa, Artificial Sequence
  Description of Artificial Sequence Synthetic HM1R3M1 recombinant polypeptide
MGSSHHHHHHSSGLVPRGSHMKKKRKAAVLLPVLLAAPRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYAAVLLPVLLAAP

SEQ ID NO: 204 - DNA, 273 bp, Artificial Sequence
  Description of Artificial Sequence Synthetic HR3NM1 polynucleotide
atgggcagcagccatcatcatcatcatcacagcagcggcctggtgccgcgcggcagccatatgaagaagaagaggaagcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgcgcagccgttcttctccctgttcttcttgccgcaccctga

SEQ ID NO: 205 - protein, 90 aa, Artificial Sequence
  Description of Artificial Sequence Synthetic HR3NM1 recombinant polypeptide
MGSSHHHHHHSSGLVPRGSHMKKKRKRIPVDPSTSRRFTPPSPAFPCGGGGGKMGENSGALSAQAAVGPGGRARPEVRAAVLLPVLLAAP

SEQ ID NO: 206 - DNA, 504 bp, Artificial Sequence
  Description of Artificial Sequence Synthetic HR3RM1 polynucleotide
atgggcagcagccatcatcatcatcatcacagcagcggcctggtgccgcgcggcagccatatgaagaagaagaggaagtcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacgggcagccgttcttctccctgttcttcttgccgcaccctga

SEQ ID NO: 207 - protein, 167 aa, Artificial Sequence
  Description of Artificial Sequence Synthetic HR3RM1 recombinant polypeptide
MGSSHHHHHHSSGLVPRGSHMKKKRKSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRAAVLLPVLLAAP

SEQ ID NO: 208 - DNA, 816 bp, Artificial Sequence
  Description of Artificial Sequence Synthetic HR3PM1 polynucleotide
atgggcagcagccatcatcatcatcatcacagcagcggcctggtgccgcgcggcagccatatgaagaagaagaggaagcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacgcagccgttcttctccctgttcttcttgccgcaccctga

SEQ ID NO: 209 - protein, 271 aa, Artificial Sequence
  Description of Artificial Sequence Synthetic HR3PM1 recombinant polypeptide
MGSSHHHHHHSSGLVPRGSHMKKKRKHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYAAVLLPVLLAAP

SEQ ID NO: 210 - DNA, 660 bp, Artificial Sequence
  Description of Artificial Sequence Synthetic HR3NRM1 polynucleotide
atgggcagcagccatcatcatcatcatcacagcagcggcctggtgccgcgcggcagccatatgaagaagaagaggaagcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacgggcagccgttcttctccctgttcttcttgccgcaccctga

SEQ ID NO: 211 - protein, 219 aa, Artificial Sequence
  Description of Artificial Sequence Synthetic HR3NRM1 recombinant polypeptide
MGSSHHHHHHSSGLVPRGSHMKKKRKRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRAAVLLPVLLAAP

SEQ ID NO: 212 - DNA, 1203 bp, Artificial Sequence
  Description of Artificial Sequence Synthetic HR3RPM1 polynucleotide
atgggcagcagccatcatcatcatcatcacagcagcggcctggtgccgcgcggcagccatatgaagaagaagaggaagtcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacgcagccgttcttctccctgttcttcttgccgcaccctga

SEQ ID NO: 213 - protein, 400 aa, Artificial Sequence
  Description of Artificial Sequence Synthetic HR3RPM1 recombinant polypeptide
MGSSHHHHHHSSGLVPRGSHMKKKRKSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYAAVLLPVLLAAP

SEQ ID NO: 214 - DNA, 516 bp, Artificial Sequence
  Description of Artificial Sequence Synthetic HR3CRM1 polynucleotide
atgggcagcagccatcatcatcatcatcacagcagcggcctggtgccgcgcggcagccatatgaagaagaagaggaagcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacgcagccgttcttctccctgttcttcttgccgcaccctga

SEQ ID NO: 215 - protein, 171 aa, Artificial Sequence
  Description of Artificial Sequence Synthetic HR3CRM1 recombinant polypeptide
MGSSHHHHHHSSGLVPRGSHMKKKRKRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDAAVLLPVLLAAP

SEQ ID NO: 216 - DNA, 1353 bp, Artificial Sequence
  Description of Artificial Sequence Synthetic HM2R3 polynucleotide
atgggcagcagccatcatcatcatcatcacagcagcggcctggtgccgcgcggcagccatatgaagaagaagaggaagctgattgcgctgctggcggcgccgctggcgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactga

SEQ ID NO: 217 - protein, 450 aa, Artificial Sequence
  Description of Artificial Sequence Synthetic HM2R3 recombinant polypeptide
MGSSHHHHHHSSGLVPRGSHMKKKRKLIALLAAPLARIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 218 - DNA, 1353 bp, Artificial Sequence
  Description of Artificial Sequence Synthetic HR3M2 polynucleotide
atgggcagcagccatcatcatcatcatcacagcagcggcctggtgccgcgcggcagccatatgaagaagaagaggaagcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacctgattgcgctgctggcggcgccgctggcgtga

SEQ ID NO: 219 - protein, 450 aa, Artificial Sequence
  Description of Artificial Sequence Synthetic HR3M2 recombinant polypeptide
MGSSHHHHHHSSGLVPRGSHMKKKRKRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYLIALLAAPLA

SEQ ID NO: 220 - DNA, 1383 bp, Artificial Sequence
  Description of Artificial Sequence Synthetic HM2R3M2 polynucleotide
atgggcagcagccatcatcatcatcatcacagcagcggcctggtgccgcgcggcagccatatgaagaagaagaggaagctgattgcgctgctggcggcgccgctggcgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacctgattgcgctgctggcggcgccgctggcgtga

SEQ ID NO: 221 - protein, 460 aa, Artificial Sequence
  Description of Artificial Sequence Synthetic HM2R3M2 recombinant polypeptide
MGSSHHHHHHSSGLVPRGSHMKKKRKLIALLAAPLARIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYLIALLAAPLA

SEQ ID NO: 222 - DNA, 1356 bp, Artificial Sequence
  Description of Artificial Sequence Synthetic HM3R3 polynucleotide
atgggcagcagccatcatcatcatcatcacagcagcggcctggtgccgcgcggcagccatatgaagaagaagaggaagctgctggcggcggcggcggcgctgctgctggcgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactga

SEQ ID NO: 223 - protein, 451 aa, Artificial Sequence
  Description of Artificial Sequence Synthetic HR3RPM1 recombinant polypeptide
MGSSHHHHHHSSGLVPRGSHMKKKRKLLAAAAALLLARIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 224 - DNA, 1356 bp, Artificial Sequence
  Description of Artificial Sequence Synthetic HR3M3 polynucleotide
atgggcagcagccatcatcatcatcatcacagcagcggcctggtgccgcgcggcagccatatgaagaagaagaggaagcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacctgctggcggcggcggcggcgctgctgctggcgtga

SEQ ID NO: 225 - protein, 451 aa, Artificial Sequence
  Description of Artificial Sequence Synthetic HR3M3 recombinant polypeptide
MGSSHHHHHHSSGLVPRGSHMKKKRKRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYLLAAAAALLLA

SEQ ID NO: 226 - DNA, 1389 bp, Artificial Sequence
  Description of Artificial Sequence Synthetic HM3R3M3 polynucleotide
atgggcagcagccatcatcatcatcatcacagcagcggcctggtgccgcgcggcagccatatgaagaagaagaggaagctgctggcggcggcggcggcgctgctgctggcgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacctgctggcggcggcggcggcgctgctgctggcgtga

SEQ ID NO: 227 - protein, 462 aa, Artificial Sequence
  Description of Artificial Sequence Synthetic HM3R3M3 recombinant polypeptide
MGSSHHHHHHSSGLVPRGSHMKKKRKLLAAAAALLLARIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYLLAAAAALLLA

SEQ ID NO: 228 - DNA, 1356 bp, Artificial Sequence
  Description of Artificial Sequence Synthetic HM4R3 polynucleotide
atgggcagcagccatcatcatcatcatcacagcagcggcctggtgccgcgcggcagccatatgaagaagaagaggaagctggcggcggcggcgctggcggtgctgccgctgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactga

SEQ ID NO: 229 - protein, 451 aa, Artificial Sequence
  Description of Artificial Sequence Synthetic HM4R3 recombinant polypeptide
MGSSHHHHHHSSGLVPRGSHMKKKRKLAAAALAVLPLRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 230 - DNA, 1356 bp, Artificial Sequence
  Description of Artificial Sequence Synthetic HR3M4 polynucleotide
atgggcagcagccatcatcatcatcatcacagcagcggcctggtgccgcgcggcagccatatgaagaagaagaggaagcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacctggcggcggcggcgctggcggtgctgccgctgtga

SEQ ID NO: 231 - protein, 451 aa, Artificial Sequence
  Description of Artificial Sequence Synthetic HR3M4 recombinant polypeptide
MGSSHHHHHHSSGLVPRGSHMKKKRKRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYLAAAALAVLPL

SEQ ID NO: 232 - DNA, 1389 bp, Artificial Sequence
  Description of Artificial Sequence Synthetic HM4R3M4 polynucleotide
atgggcagcagccatcatcatcatcatcacagcagcggcctggtgccgcgcggcagccatatgaagaagaagaggaagctggcggcggcggcgctggcggtgctgccgctgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacctggcggcggcggcgctggcggtgctgccgctgtga

SEQ ID NO: 233 - protein, 462 aa, Artificial Sequence
  Description of Artificial Sequence Synthetic HM4R3M4 recombinant polypeptide
MGSSHHHHHHSSGLVPRGSHMKKKRKLAAAALAVLPLRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYLAAAALAVLPL

SEQ ID NO: 234 - DNA, 1350 bp, Artificial Sequence
  Description of Artificial Sequence Synthetic HM5R3 polynucleotide
atgggcagcagccatcatcatcatcatcacagcagcggcctggtgccgcgcggcagccatatgaagaagaagaggaaggcgctgctggcggcgctgctggcgccgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactga

SEQ ID NO: 235 - protein, 449 aa, Artificial Sequence
  Description of Artificial Sequence Synthetic HM5R3 recombinant polypeptide
MGSSHHHHHHSSGLVPRGSHMKKKRKALLAALLAPRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 236 - DNA, 1350 bp, Artificial Sequence
  Description of Artificial Sequence Synthetic HR3M5 polynucleotide
atgggcagcagccatcatcatcatcatcacagcagcggcctggtgccgcgcggcagccatatgaagaagaagaggaagcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacgcgctgctggcggcgctgctggcgccgtga

SEQ ID NO: 237 - protein, 449 aa, Artificial Sequence
  Description of Artificial Sequence Synthetic HR3M5 recombinant polypeptide
MGSSHHHHHHSSGLVPRGSHMKKKRKRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYALLAALLAP

SEQ ID NO: 238 - DNA, 1377 bp, Artificial Sequence
  Description of Artificial Sequence Synthetic HM5R3M5 polynucleotide
atgggcagcagccatcatcatcatcatcacagcagcggcctggtgccgcgcggcagccatatgaagaagaagaggaaggcgctgctggcggcgctgctggcgccgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctacgcgctgctggcggcgctgctggcgccgtga

SEQ ID NO: 239 - protein, 458 aa, Artificial Sequence
  Description of Artificial Sequence Synthetic HM5R3M5 recombinant polypeptide
MGSSHHHHHHSSGLVPRGSHMKKKRKALLAALLAPRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYALLAALLAP

SEQ ID NO: 240 - DNA, 1323 bp, Artificial Sequence
  Description of Artificial Sequence Synthetic HR3 polynucleotide
atgggcagcagccatcatcatcatcatcacagcagcggcctggtgccgcgcggcagccatatgaagaagaagaggaagcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactga

SEQ ID NO: 241 - protein, 440 aa, Artificial Sequence
  Description of Artificial Sequence Synthetic HR3 recombinant polypeptide
MGSSHHHHHHSSGLVPRGSHMKKKRKRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 242 - DNA, 51 bp, Artificial Sequence
  Description of Artificial Sequence Synthetic HR-5′ forward primer
ccgcatatgaagaagaagaggaagcgtattcccgtagacccaagcaccagc

SEQ ID NO: 243 - DNA, 33 bp, Artificial Sequence
  Description of Artificial Sequence Synthetic HR-3′ reverse primer
ccgcatatgtcagtagggccgccacacggcctc

SEQ ID NO: 244 - DNA, 87 bp, Artificial Sequence
  Description of Artificial Sequence Synthetic HM1R-5′ forward primer
ccgcatatgaagaagaagaggaaggcagccgttcttctccctgttcttcttgccgcaccccgtattcccgtagacccaagcaccagc

SEQ ID NO: 245 - DNA, 75 bp, Artificial Sequence
  Description of Artificial Sequence Synthetic HRM1-3′ reverse primer
ccgcatatgtcagggtgcggcaagaagaacagggagaagaacggctgcgtagggccgccacacggcctcatccat

SEQ ID NO: 246 - DNA, 51 bp, Artificial Sequence; identical to SEQ ID NO: 242
  Description of Artificial Sequence Synthetic HR-N-5′ forward primer

SEQ ID NO: 247 - DNA, 75 bp, Artificial Sequence
  Description of Artificial Sequence Synthetic HR-N-M1-3′ reverse primer
ccgcatatgtcagggtgcggcaagaagaacagggagaagaacggctgcgcgcacctcgggccgggcgcgccctcc

SEQ ID NO: 248 - DNA, 51 bp, Artificial Sequence
  Description of Artificial Sequence Synthetic HR-R-5′ forward primer
ccgcatatgaagaagaagaggaagtcgatggtggacgtgctggcggaccac

SEQ ID NO: 249 - DNA, 75 bp, Artificial Sequence
  Description of Artificial Sequence Synthetic HR-R-M1-3′ reverse primer
ccgcatatgtcagggtgcggcaagaagaacagggagaagaacggctgcccgtctgggctcccggggtccgtccac

SEQ ID NO: 250 - DNA, 51 bp, Artificial Sequence
  Description of Artificial Sequence Synthetic HR-P-5′ forward primer
ccgcatatgaagaagaagaggaagcaccggcagaagctggaggaccagacc

SEQ ID NO: 251 - DNA, 51 bp, Artificial Sequence
  Description of Artificial Sequence Synthetic HR-CR-5′ forward primer
ccgcatatgaagaagaagaggaagcgcaccgacagccccaacttcctctgc

SEQ ID NO: 252 - DNA, 75 bp, Artificial Sequence
  Description of Artificial Sequence Synthetic HR-CR-M1-3′ reverse primer
ccgcatatgtcagggtgcggcaagaagaacagggagaagaacggctgcgtccccaaagcggtcagggaacggctt

SEQ ID NO: 253 - DNA, 81 bp, Artificial Sequence
  Description of Artificial Sequence Synthetic HM2R-5′ forward primer
ccgcatatgaagaagaagaggaagctgattgcgctgctggcggcgccgctggcgcgtattcccgtagacccaagcaccagc

SEQ ID NO: 254 - DNA, 69 bp, Artificial Sequence
  Description of Artificial Sequence Synthetic HRM2-3′ reverse primer
ccgcatatgtcacgccagcggcgccgccagcagcgcaatcaggtagggccgccacacggcctcatccat

SEQ ID NO: 255 - DNA, 84 bp, Artificial Sequence
  Description of Artificial Sequence Synthetic HM3R-5′ forward primer
ccgcatatgaagaagaagaggaagctgctggcggcggcggcggcgctgctgctggcgcgtattcccgtagacccaagcaccagc

SEQ ID NO: 256 - DNA, 72 bp, Artificial Sequence
  Description of Artificial Sequence Synthetic HRM3-3′ reverse primer
ccgcatatgtcacgccagcagcagcgccgccgccgccgccagcaggtagggccgccacacggcctcatccat

SEQ ID NO: 257 - DNA, 84 bp, Artificial Sequence
  Description of Artificial Sequence Synthetic HM4R-5′ forward primer
ccgcatatgaagaagaagaggaagctggcggcggcggcgctggcggtgctgccgctgcgtattcccgtagacccaagcaccagc

SEQ ID NO: 258 - DNA, 72 bp, Artificial Sequence
  Description of Artificial Sequence Synthetic HRM4-3′ reverse primer
ccgcatatgtcacagcggcagcaccgccagcgccgccgccgccaggtagggccgccacacggcctcatccat

SEQ ID NO: 259 - DNA, 78 bp, Artificial Sequence
  Description of Artificial Sequence Synthetic HM5R-5′ forward primer
ccgcatatgaagaagaagaggaaggcgctgctggcggcgctgctggcgccgcgtattcccgtagacccaagcaccagc

SEQ ID NO: 260 - DNA, 66 bp, Artificial Sequence
  Description of Artificial Sequence Synthetic HRM5-3′ reverse primer
ccgcatatgtcacggcgccagcagcgccgccagcagcgcgtagggccgccacacggcctcatccat

SEQ ID NO: 261 - protein, 6 aa, Artificial Sequence
  Description of Artificial Sequence Synthetic 6xHis tag
HHHHHH